(19) United States
(12) Patent Application Publication
(10) Pub. No.: US 2007/0135454 A1
(43) Pub. Date: Jun. 14, 2007

(54) Bicyclic pyrimidin-4(3h)-ones and analogues and derivatives thereof which modulate the function of the vanilloid-1-receptor(vr1)

(76) Inventors: Tracy Bayliss, London (GB); Gregory John Hollingworth, Brentwood (GB); Rebecca Elizabeth Jarvis, Cambridge (GB); Brian A. Jones, Saffron Walden (GB); Timothy Jason Sparey, London (GB)

Correspondence Address: MERCK AND CO., INC, P O BOX 2000, RAHWAY, NJ, 07065-0907, US

(21) Appl. No.: 10/578,855

(22) PCT Filed: Nov. 12, 2004
(86) PCT No.: PCT/GB04/04816

§ 371 Date: May 11, 2006

(30) Foreign Application Priority Data
Nov. 14, 2003 (GB) 0326634.3
Apr. 14, 2004 (GB) 0408348.1

(51) Int. Cl. A61K 31/519; C07D 498/02; C07D 491/02; C07D 487/02

(52) U.S. Cl. 514/260.1; 514/262.1; 514/265.1; 544/262; 544/278; 544/279; 544/280

(57) ABSTRACT

Compounds of formula (I); which are useful as therapeutic compounds, particularly in the treatment of pain and other conditions ameliorated by the modulation of the function of the vanilloid-1 receptor (VR1).

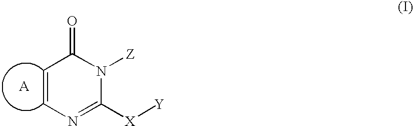

The present invention is concerned with 2,3-substituted fused bicyclic pyrimidin-4(3H)-ones and analogues and derivatives thereof as well as pharmaceutically acceptable salts and prodrugs thereof, which are useful as therapeutic compounds, particularly in the treatment of pain and other conditions ameliorated by the modulation of the function of the vanilloid-1 receptor (VR1).

The pharmacologically active ingredient of chilli peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibers that ultimately generate the sensation of pain. Importantly the VR1 receptor is activated not only by capsaicin but also by acidic pH and by noxious heat stimuli. It is also sensitized by a number of inflammatory mediators and thus appears to be a polymodal integrator of painful stimuli.

The prototypical VR1 antagonist is capsazepine (Walpole et al., J. Med. Chem., 37:1942, 1994)—VR1 IC₅₀ of 420 nM. A novel series of sub-micromolar antagonists has also been reported recently (Lee et al, Bioorg. Med. Chem., 9:1713, 2001), but these reports provide no evidence for in vivo efficacy. A much higher affinity antagonist has been derived from the ‘ultra-potent’ agonist resiniferatoxin. Iodo-resiniferatoxin (Wahl et al., Mol. Pharmacol, 59:9, 2001) is a nanomolar antagonist of VR1 but does not possess properties suitable for an oral pharmaceutical. This last is also true of the micromolar peptoid antagonists described by Garcia-Martinez (Proc. Natl. Acad Sci., USA, 99:2374, 2002).

EP-A-0807633 (Pfizer Inc.) discloses structurally related AMPA receptor antagonists for treating neurodegenerative and CNS-trauma related conditions.

WO-A-9733890 (Novartis AG) discloses structurally related compounds as pesticides.

The compounds of the present invention have advantageous properties, such as good metabolic stability.

We herein describe another novel series of VR1 modulators. These comprise predominantly VR1 antagonists but encompass VR1 partial antagonists and VR1 partial agonists. Such compounds have been shown to be efficacious in animal models of pain.

The present invention provides compounds of formula I: 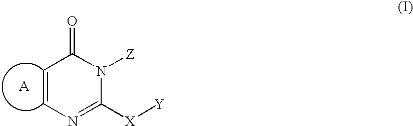
wherein:

A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;

A is optionally substituted by one, two or three groups independently chosen from halogen, hydroxy, S(O)ᵣC₁₋₄alkyl, S(O)ᵣNR⁵R⁶, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl; and a phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁵R⁶ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;

X is O, S or NR¹ where R¹ is hydrogen or C₁₋₆alkyl;

Y is (CR²R³)ₙ(CO)ₚ(NR⁴)_{q}W;

R² and R³ are independently hydrogen, hydroxy, halogen or C₁₋₄alkyl;

R⁴ is hydrogen or C₁₋₆alkyl;

n is zero, one, two, three or four;

p is zero or one;

q is zero or one;

r is zero, one or two;

W is hydrogen, C₁₋₆alkoxy, haloC₁₋₆alkoxy, C₁₋₆alkyl, haloC₁₋₆alkyl, hydroxyC₁₋₆alkyl, aminoC₁₋₆alkyl, carboxyC₁₋₆alkyl, C₃₋₇cycloalkyl, haloC₃₋₇cycloalkyl; or a phenyl ring, a five-membered heteroaromatic ring containing one, two, three or four heteroatoms independently chosen from O, N and S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, a six-membered saturated ring containing one or two heteroatoms independently chosen from O and N, the ring being optionally substituted by halogen, C₁₋₆alkyl, C₂₋₆alkenyl, C₂₋₆alkynyl, nitro, cyano, C₃₋₇cycloalkyl, hydroxy, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described or NR⁵R⁶;

each R⁵ and R⁶ is independently hydrogen or C₁₋₆alkyl or R⁵ and R⁶, together with the nitrogen atom to which they are attached, may form a saturated 4-7 membered ring;

Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by halogen, hydroxy, cyano, nitro, NR⁵R⁶ as defined above, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;

when R¹ and R⁴ are alkyl groups they may, together with the nitrogen atoms to which they are attached, form a piperazine ring;

or a pharmaceutically acceptable salt or tautomer thereof.

In one embodiment of the compounds of formula I R² and R³ are not hydroxy and n is not zero.

In another embodiment of the compounds of formula I:

A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;

A is optionally substituted by one, two or three groups independently chosen from halogen, hydroxy, phenyl, S(O)ᵣC₁₋₄alkyl, S(O)ᵣNR⁵R⁶, formyl, C₁₋₄alkylcarbonyl, C₁₋₆alkyl, haloC₁₋₆alkyl, hydroxyC₁₋₆alkyl, C₁₋₆ alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl and aminoC₁₋₆alkoxy;

X is O, S or NR¹ where R¹ is hydrogen or C₁₋₆alkyl;

Y is (CR²R³)ₙ(CO)ₚ(NR⁴)_{q}W;

R² and R³ are independently hydrogen, halogen or C₁₋₄alkyl;

R⁴ is hydrogen or C₁₋₆alkyl;

n is one, two, three or four;

p is zero or one;

q is zero or one;

r is zero, one or two;

W is hydrogen, C₁₋₆alkoxy, C₁₋₆alkyl; or a phenyl ring, a five-membered heteroaromatic ring containing one, two, three or four heteroatoms independently chosen from O, N and S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, the ring being optionally substituted by halogen, C₁₋₆alkyl, C₂₋₆alkenyl, C₂₋₆alkynyl, nitro, cyano, C₃₋₇cycloalkyl, hydroxy, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described or NR⁵R⁶;

each R⁵ and R⁶ is independently hydrogen or C₁₋₆alkyl or R⁵ and R⁶, together with the nitrogen atom to which they are attached, may form a saturated 4-7 membered ring;

Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by halogen, hydroxy, cyano, nitro, NR⁵R⁶ as defined above, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;

when R¹ and R⁴ are alkyl groups they may, together with the nitrogen atoms to which they are attached, form a piperazine ring;

or a pharmaceutically acceptable salt thereof

A may be a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms.

A is preferably unsubstituted or substituted by halogen, hydroxy, C₃₋₆cycloalkyl, C₁₋₄alkyl, haloC₁₋₄alkyl, C₁₋₄alkoxy, haloC₁₋₄alkoxy or phenyl. A further preferred substituent is hydroxyC₁₋₄alkyl, aminoC₁₋₄alkyl, C₁₋₄alkylaminoC₁₋₄alkyl or di(C₁₋₄alkyl)aminoC₁₋₄alkyl.

A is preferably unsubstituted or substituted by halogen, hydroxy, C₃₋₅cycloalkyl, C₁₋₄alkyl, haloC₁₋₄alkyl, C₁₋₄alkoxy or haloC₁₋₄alkoxy. More preferably A is unsubstituted or substituted by halogen or C₁₋₄alkyl. A is preferably unsubstituted or substituted by methyl. Favourably A is unsubstituted or substituted by methyl, ethyl, cyclopropyl or phenyl. In one embodiment A is not thiophene.

More particularly A is unsubstituted or substituted by methyl, ethyl, proply, isopropyl, hydroxyethyl, cyclopropyl, cyclopropylmethyl, phenyl or dimethylaminoethyl.

A is preferably a fused pyridine, thiophene, thiazole or imidazole.

When A is substituted by hydroxy group tautomerism may occur. For example when A is fused imidazole, tautomerism may occur to form an imidazolone.

X may be O. X may be S. X may be NH.

R¹ is preferably hydrogen or C₁₋₂alkyl. R¹ may be hydrogen.

R² and R³ are preferably hydrogen, halogen, methyl or hydroxy.

R² and R³ are preferably hydrogen, halogen or methyl. R² and R³ are most preferably hydrogen. Suitably, R² and R³ are independently selected from hydrogen, hydroxy and methyl.

R⁴ is preferably hydrogen or C₁₋₂alkyl. R⁴ may be hydrogen.

R₁ and R⁴, together with the nitrogen atoms to which they are attached, may form a piperazine ring, such as a piperazinone ring.

n is preferably one, two, three or four.

n is preferably one, two or three.

n is preferably one or two.

In one embodiment p is zero. In another embodiment p is one.

In one embodiment q is zero. In another embodiment q is one.

Particular embodiments of (CR²R³)ₙ(CO)ₚ(NR⁴)_{q} include CH₂, CH₂CO, CH₂CH₂ and CH₂CONH. In one embodiment (CR²R³)ₙ(CO)ₚ(NR⁴)_{q} is CH₂CH₂CH₂. In further embodiments (CR²R³)ₙ(CO)ₚ(NR⁴)_{q} is CH₂CH₂CH₂CH₂, CH₂CH(OH), CH₂C(OH)₂, CH₂CON(CH₃) or a direct bond.

W is preferably hydrogen, C₁₋₆alkyl, haloC₁₋₆alkyl or C₃₋₇cycloalkyl. A further preferred W group is C₁₋₆alkoxy.

In one embodiment W is not hydrogen or C₁₋₆alkyl.

Preferably W is an aromatic ring as defined above.

W is preferably unsubstituted or substituted by halogen, C₁₋₄alkyl, hydroxy, C₁₋₄alkoxy, haloC₁₋₄alkyl, phenyl, haloC₁₋₄alkoxy or NR⁵R⁶ where R⁵ and R⁶ are independently C₁₋₄alkyl or, R⁵ and R⁶, together with the nitrogen atom to which they are attached, form a 5-6 membered saturated ring. More preferably W is unsubstituted or substituted by halogen, C₁₋₂alkyl, C₁₋₂haloalkyl, C₁₋₂alkoxy or phenyl. Further preferred substituents include C₁₋₂haloalkoxy and hydroxy. If substituted W is preferably monosubstituted. W may be disubstituted. Particular substituents include fluorine, chlorine, trifluoromethoxy, trifluoromethyl, pyrrolidine, methyl and phenyl. Further particular substituents are hydroxy and methoxy.

Particular aromatic W rings include benzene, benzothiazole, benzothiophene, pyridine, 1,2,4-oxadiazole and isoxazole. A further aromatic W ring is thiazole.

Particular embodiments of W include methyl, 3-fluorophenyl, 4-chlorophenyl, 5-chloro-1-benzothien-3-yl, 1-benzothien-3-yl, 1,3-benzothiazol-2-yl, phenyl, 3-chlorophenyl, 4-trifluoromethoxyphenyl, 4-trifluoromethylphenyl, 4-pyrrolidin-1-ylphenyl, pyrid-2-yl, 4-fluorophenyl, 5-phenyl-1,2,4-oxadiazol-3-yl and 5-methylisoxazol-3-yl. Further embodiments of W include hydrogen, cyclopropyl, cyclohexyl, trifluoromethyl, 2-fluoro-4-trifluoromethylphenyl.

Additional embodiments of W include 2-chlorophenyl, 2-fluorophenyl, 6-chloro-1-benzothien-3-yl, 3,4-dichlorophenyl, 2,4-dichlorophenyl, 3-chloro-4-fluorophenyl, 3-fluoro-4-trifluoromethylphenyl, ethoxy, 3-trifluoromethylphenyl, 2-hydroxy-4-trifluoromethylphenyl, 2-chloro-4-trifluoromethylphenyl, 5-trifluoromethyl-1,3-benzothiazol-2-yl, 5-chloro-1,3-benzothiazol-2-yl, cyclobutyl, cyclopentyl, 2-methyl-1,3-thiazol-4-yl, fluoromethyl, 4-trifluoromethyl-1,3-thiazol-2-yl, 6-trifluoromethylpyridin-3-yl, 2-trifluoromethyl-1,3-thiazol-4-yl, ethyl, 3-trifluoromethylpyridin-2-yl, 2-methoxy-4-trifluoromethylphenyl and isopropyl.

Z is preferably an optionally substituted phenyl or pyridinyl.

Z is preferably unsubstituted or substituted by one or two substituents chosen from cyano, halogen, C₁₋₄alkyl, haloC₁₋₄alkyl, C₁₋₄alkoxy, haloC₁₋₄alkoxy, amino, C₁₋₄alkylamino and di(C₁₋₄alkyl)amino. Particular substituents include chlorine, trifluoromethyl, cyano, methyl, fluorine, ethoxy, trifluoromethoxy, bromine, dimethylamino, methoxy and isopropoxy.

Particular embodiments of Z include 4-chlorophenyl, 4-trifluoromethylphenyl, 4-cyanophenyl, 4-methylphenyl, phenyl, 6-chloropyridin-3-yl, 3-chlorophenyl, 4-fluorophenyl, 4-chloro-3-ethoxyphenyl, 4-trifluoromethoxyphenyl, 2-fluoro-4-trifluoromethylphenyl, 4-bromophenyl, 4-dimethylaminophenyl, 2,4-dichlorophenyl, 3-chloro-4-fluorophenyl, 3,4-difluorophenyl, 3-fluoro-4-methylphenyl, 4-chloro-2-fluorophenyl, 4-chloro-3-fluorophenyl, 4-chloro-3-methoxyphenyl, 3-bromo-4-chlorophenyl, 4-chloro-3-isopropoxyphenyl and 4-chloro-3-cyanophenyl.

Z is preferably unsubstituted or substituted by one substituent chosen from cyano, halogen, C₁₋₄alkyl, haloC₁₋₄alkyl, C₁₋₄alkoxy and haloC₁₋₄alkoxy. Z is preferably monosubstituted. Z is preferably a phenyl ring. Preferred substituents are chlorine and trifluoromethyl. Particular embodiments of Z are 4-chlorophenyl and 4-trifluoromethylphenyl. In one embodiment Z is not substituted by trifluoromethyl.

In another embodiment Z is substituted by cyano or methyl. Thus said Z can be cyanophenyl or methylphenyl. Particularly Z is 4-methylphenyl or 4-cyanophenyl.

The present invention also provides compounds of formula (I)¹: 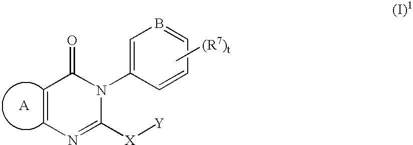
wherein:

B is N or CH;

t is 1, 2 or 3;

A is a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;

A is optionally substituted by halogen, C₁₋₄alkyl, hydroxyC₁₋₄alkyl; C₃₋₇cycloalkyl, phenyl or di(C₁₋₄alkyl)aminoC₁₋₄alkyl;

X is O, S or NR¹ where R¹ is hydrogen or C₁₋₄alkyl;

Y is (CR²R³)ₙ(CO)ₚ(NR⁴)_{q}W, where R², R³, R⁴, n, p and q are as defined for formula I;

W is hydrogen, C₁₋₆alkoxy, C₁₋₆alkyl, haloC₁₋₆alkyl, C₃₋₇cycloalkyl; or a phenyl ring, a five-membered heteroaromatic ring containing one, two, three or four heteroatoms independently chosen from O, N or S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, the ring being optionally substituted by halogen, C₁₋₄alkyl, hydroxy, C₁₋₄alkoxy, haloC₁₋₄alkyl, phenyl, haloC₁₋₄alkoxy or NR⁵R⁶ where R⁵ and R⁶ are independently C₁₋₄alkyl or, R⁵ and R⁶, together with the nitrogen atom to which they are attached, form a 5-6 membered saturated ring;

R⁷ is hydrogen, cyano, halogen, C₁₋₄alkyl, haloC₁₋₄alkyl, C₁₋₄alkoxy, haloC₁₋₄alkoxy, amino, C₁₋₄alkylamino or di(C₁₋₄alkyl)amino;

when R¹ and R⁴ are alkyl groups they may, together with the nitrogen atoms to which they are attached, form a piperazine ring; or a pharmaceutically acceptable salt or tautomer thereof.

R⁷ is preferably hydrogen, chlorine, trifluoromethyl, cyano, methyl, fluorine, ethoxy, trifluoromethoxy, bromine, dimethylamino, methoxy and isopropoxy.

In one embodiment B is CH. In another embodiment B is N.

The present invention also provides compounds of formula IA:

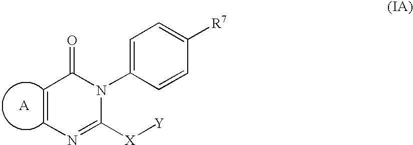

wherein A is a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;

A is optionally substituted by halogen, C₁₋₄alkyl, C₃₋₇cycloalkyl or phenyl;

X is O, S or NR¹ where R¹ is hydrogen or C₁₋₄alkyl;

Y is (CR²R³)ₙ(CO)ₚ(NR⁴)_{q}W, where R², R³, R⁴, n, p and q are as defined for formula I;

W is hydrogen, C₁₋₆ alkyl, haloC₁₋₆alkyl, C₃₋₇cycloalkyl; or a phenyl ring, a five-membered heteroaromatic ring containing one, two, three or four heteroatoms independently chosen from O, N or S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, the ring being optionally substituted by halogen, C₁₋₄alkyl, hydroxy, C₁₋₄alkoxy, haloC₁₋₄alkyl, phenyl, haloC₁₋₄alkoxy or NR⁵R⁶ where R⁵ and R⁶ are independently C₁₋₄alkyl or, R⁵ and R⁶, together with the nitrogen atom to which they are attached, form a 5-6 membered saturated ring;

R⁷ is hydrogen, cyano, halogen, C₁₋₄alkyl, haloC₁₋₄alkyl, C₁₋₄alkoxy or haloC₁₋₄alkoxy;

when R¹ and R⁴ are alkyl groups they may, together with the nitrogen atoms to which they are attached, form a piperazine ring;

or a pharmaceutically acceptable salt or tautomer thereof.

In one embodiment A is optionally substituted by halogen or C₁₋₄alkyl.

A is preferably a fused pyridine, thiophene, thiazole or imidazole which is unsubstituted or substituted by methyl, ethyl, propyl, isopropyl, hydroxyethyl, cyclopropyl, cycloproplymethyl, phenyl or dimethylaminoethyl.

A is preferably a fused pyridine, thiophene, thiazole or imidazole which is unsubstituted or substituted by halogen, methyl, ethyl, cyclopropyl or phenyl.

A is preferably a fused pyridine, thiophene, thiazole or imidazole which is unsubstituted or substituted by halogen or methyl.

R¹ is preferably hydrogen or C₁₋₂alkyl, most preferably hydrogen.

Y is preferably CH₂W, CH₂COW, CH₂CH₂W or CH₂CONHW, or X—Y is

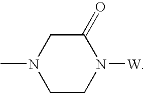

Y is preferably CH₂CH₂CH₂W.

Further preferred embodiments of Y include CH₂CH₂CH₂CH₂W, CH₂CH(OH)W, CH₂C(OH)₂W, CH₂CON(CH₃)W and W.

Preferably X—Y is

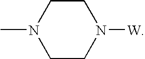

In one embodiment W is C₁₋₆ alkyl; or a phenyl ring, a five-membered heteroaromatic ring containing one, two, three or four heteroatoms independently chosen from O, N or S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, the ring being optionally substituted by halogen, C₁₋₄alkyl, hydroxy, C₁₋₄alkoxy, haloC₁₋₄alkyl, phenyl, haloC₁₋₄alkoxy or NR⁵R⁶ where R₅ and R⁶ are independently C₁₋₄alkyl or, R⁵ and R⁶, together with the nitrogen atom to which they are attached, form a 5-6 membered saturated ring.

W is preferably unsubstituted or substituted by halogen, C₁₋₂alkyl, C₁₋₂haloalkyl, C₁₋₂alkoxy or phenyl. Further preferred substituents include C₁₋₂haloalkoxy and hydroxy. More preferably W is unsubstituted or monosubstituted by fluorine, chlorine, trifluoromethoxy, trifluoromethyl, pyrrolidine, methyl or phenyl.

Preferably W is unsubstituted, monosubstituted or disubstituted by a group independently selected from fluorine, chlorine, trifluoromethoxy, trifluoromethyl, pyrrolidine, methyl and phenyl. Further preferred substituents are hydroxy and methoxy.

W is preferably a benzene, benzothiazole, benzothiophene, pyridine, 1,2,4-oxadiazole or isoxazole ring. A further preferred ring is thiazole. Preferably W is hydrogen, methyl, trifluoromethyl, cyclopropyl or cyclohexyl. Further preferred W groups include fluoromethyl, ethoxy, cyclobutyl, cyclopentyl, ethyl and isopropyl.

R⁷ is preferably chlorine or trifluoromethyl. In one embodiment R⁷ is not trifluoromethyl. In another embodiment R⁷ is cyano or methyl.

Particular embodiments of the invention include:
3-(4-chlorophenyl)-2-[3-fluorobenzylthio]pyrido[3,4-d]pyrimidin-4(3H)-one;
3-(4-chlorophenyl)-2-{2-(4-chlorophenyl)-2-oxoethylthio}pyrido[3,2-d]pyrimidin-4(3H)-one;
3-(4-chlorophenyl)-2-[3-fluorobenzylthio]pyrido[3,2-d]pyrimidin-4(3H)-one;
3-(4-chlorophenyl)-2-{2-(4-chlorophenyl)ethylthio}pyrido[3,2-d]pyrimidin-4(3H)-one;
2-{5-chloro-1-benzothien-3-ylmethylthio}-3-(4-chlorophenyl)pyrido[3,2-d]pyrimidin-4(3H)-one;
2-[1-benzothien-3-ylmethyl)thiol]-3-(4-chlorophenyl)pyrido[3,2-d]pyrimidin-4(3H)-one;
2-[1,3-benzothiazol-2-ylmethylthio]-3-(4-chlorophenyl)pyrido[3,2-d]pyrimidin-4(3H)-one;
3-(4-chlorophenyl)-2-[2-oxo-2-phenylethylthio]pyrido[3,2-d]pyrimidin-4(3H)-one;
3-(4-chlorophenyl)-2-{2-(3-chlorophenyl)-2-oxoethylthio}pyrido[3,2-d]pyrimidin-4(3H)-one;
3-(4-chlorophenyl)-2-(2-oxo-2-[4-trifluoromethoxyphenyl]ethylthio)pyrido[3,2-d]pyrimidin-4(3H)-one;
3-(4-chlorophenyl)-2-(2-oxo-2-[4-trifluoromethylphenyl]ethylthio)pyrido[3,2-d]pyrimidin-4(3H)-one;
3-(4-chlorophenyl)-2-{2-oxo-2-(4-pyrrolidin-1-ylphenyl)ethylthio}pyrido[3,2-d]pyrimidin-4(3H)-one;
3-(4-chlorophenyl)-2-[2-oxo-2-pyridin-2-ylethylthio]pyrido[3,2-d]pyrimidin-4(3H)-one;
3-(4-chlorophenyl)-2-[4-fluorobenzylthio]pyrido[3,2-d]pyrimidin-4(3H)-one;
2-[3-chlorobenzylthio]-3-(4-chlorophenyl)pyrido[3,2-d]pyrimidin-4(3H)-one;
3-(4-chlorophenyl)-2-[pyridin-2-ylmethylthio]pyrido[3,2-d]pyrimidin-4(3H)-one;
3-(4-chlorophenyl)-2-{5-phenyl-1,2,4-oxadiazol-3-ylmethylthio}pyrido[3,2-d]pyrimidin-4(3H)-one;
2-{3-(4-chlorophenyl)-4-oxo-3,4-dihydropyrido[3,2-d]pyrimidin-2-ylthio}-N-(5-methylisoxazol-3-yl)acetamide;
3-(4-chlorophenyl)-2-[3-fluorobenzylthio]thieno[2,3-d]pyrimidin-4(3H)-one;
3-(4-chlorophenyl)-2-[3-fluorobenzylthio]thieno[3,2-d]pyrimidin-4(3H)-one;
3-(4-chlorophenyl)-2-{2-(4-chlorophenyl)-2-oxoethylthio}thieno[3,2-d]pyrimidin-4(3H)-one;
6-(4-chlorophenyl)-5-[3-fluorobenzylthio][1,3]thiazolo[5,4-d]pyrimidin-7(6H)-one;
6-(4-chlorophenyl)-5-{2-(4-chlorophenyl)-2-oxoethylthio}[1,3]thiazolo[5,4-d]pyrimidin-7(6H)-one;
6-(4-chlorophenyl)-5-{2-(4-chlorophenyl)-2-oxoethylthio}[1,3]thiazolo[4,5-d]pyrimidin-7(6H)-one;
2-{5-chloro-1-benzothien-3-ylmethylthio}-1-(4-chlorophenyl)-9-methyl-1,9-dihydro-6H-purin-6-one;
1-(4-chlorophenyl)-9-methyl-2-(2-[4-trifluoromethylphenyl]ethylthio)-1,9-dihydro-6H-purin-6-one;
1-(4-chlorophenyl)-2-{2-(4-chlorophenyl)ethylthio}-9-methyl-1,9-dihydro-6H-purin-6-one;
1-(4-chlorophenyl)-2-{2-(4-chlorophenyl)-2-oxoethylthio}-9-methyl-1,9-dihydro-6H-purin-6-one;
1-(4-chlorophenyl)-2-[3-fluorobenzylthio]-9-methyl-1,9-dihydro-6H-purin-6-one;
1-(4-chlorophenyl)-2-[3-fluorobenzylthio]-1,9-dihydro-6H-purin-6-one;
2-{2-(4-chlorophenyl)-2-oxoethylthio}-3-[4-trifluoromethylphenyl]pyrido[3,2-d]pyrimidin-4(3H)-one;
2-[3-fluorobenzylthio]-3-[4-trifluoromethylphenyl]pyrido[3,2-d]pyrimidin-4(3H)-one;
2-(methylthio)-3-pyridin-3-ylpyrido[3,2-d]pyrimidin-4(3H)-one;
3-(4-chlorophenyl)-2-(3-oxo-4-phenylpiperazin-1-yl)pyrido[3,2-d]pyrimidin-4(3H)-one;
3-4-chlorophenyl-2-{2-(4-chlorophenyl)ethylamino}pyrido[3,2-d]pyrimidin-4(3H)-one;
3-(4-chlorophenyl)-2-[3-fluorobenzyloxy]thieno[3,2-d]pyrimidin-4(3H)-one; and
3-(4-chlorophenyl)-2-[3-fluorobenzylamino]thieno[3,2-d]pyrimidin-4(3H)-one;
or a pharmaceutically acceptable salt thereof

Further embodiments of this invention include:
1-(4-chlorophenyl)-2-[2-cyclohexylethylthio]-9-methyl-1,9-dihydro-6H-purin-6-one;
1-(4-chlorophenyl)-2-[2-cyclohexylethylthio]-9-ethyl-1,9-dihydro-6H-purin-6-one;
1-(4-chlorophenyl)-9-ethyl-2-[3,3,3-trifluoropropylthio]-1,9-dihydro-6H-purin-6-one;
1-(4-chlorophenyl)-9-ethyl-2-propylthio-1,9-dihydro-6H-purin-6-one;
1-(4-chlorophenyl)-2-[cyclopropylmethylthio]-9-ethyl-1,9-dihydro-6H-purin-6-one;
1-(4-chlorophenyl)-9-methyl-2-[3,3,3-trifluoropropylthio]-1,9-dihydro-6H-purin-6-one;
1-(4-chlorophenyl)-9-cyclopropyl-2-[3,3,3-trifluoropropylthio]-1,9-dihydro-6H-purin-6-one;
1-(4-chlorophenyl)-9-ethyl-2-[2,2,2-trifluoroethylthio]-1,9-dihydro-6H-purin-6-one;
1-(4-chlorophenyl)-9-ethyl-2-[4,4,4-trifluorobutylthio]-1,9-dihydro-6H-purin-6-one;
1-(4-chlorophenyl)-9-phenyl-2-(2-[4-trifluoromethylphenyl]ethylthio)-1,9-dihydro-6H-purin-6-one;
1-(4-chlorophenyl)-2-methylthio-9-phenyl-1,9-dihydro-6H-purin-6-one;
1-(4-chlorophenyl)-9-phenyl-2-[3,3,3-trifluoropropylthio]-1,9-dihydro-6H-purin-6-one;
1-(4-chlorophenyl)-9-phenyl-2-[4,4,4-trifluorobutylthiol]-1,9-dihydro-6H-purin-6-one;
4-{9-methyl-6-oxo-2-[3,3,3-trifluoropropylthio]-6,9-dihydro-1H-purin-1-yl}benzonitrile;
9-methyl-1-(4-methylphenyl)-2-[3,3,3-trifluoropropylthio]-1,9-dihydro-6H-purin-6-one;
1-(4-chlorophenyl)-9-ethyl-2-(2-[4-trifluoromethylphenyl]ethylamino)-1,9-dihydro-6H-purin-6-one; and
1-(4-chlorophenyl)-9-ethyl-2-(2-[2-fluoro-4-trifluoromethylphenyl]ethylamino)-1,9-dihydro-6H-purin-6-one;
or a pharmaceutically acceptable salt or tautomer thereof.

Further embodiments of this invention include:
3-(4-chlorophenyl)-2-methylaminothieno[3,2-d]pyrimidin-4(3H)-one;
3-(4-chlorophenyl)-2-{2-(2-chlorophenyl)-2-oxoethylthio}pyrido[3,2-d]pyrimidin-4(3H)-one;
2-{2-(4-3-(chlorophenyl)-2-oxoethylthio}-3-phenylpyrido[3,2-d]pyrimidin-4(3H)-one;
3-(4-chlorophenyl)-2-[2-phenylethylthio]pyrido[3,2-d]pyrimidin-4(3H)-one;
3-(4-chlorophenyl)-2-[2-fluorobenzylthio]pyrido[3,2-d]pyrimidin-4(3H)-one;
6-(4-chlorophenyl)-5-{2-(4-chlorophenyl)ethylthio}[1,3]thiazolo[5,4-d]pyrimidin-7(6H)-one;
3-(4-chlorophenyl)-2-{2-(3-chlorophenyl)ethylthio}pyrido[3,2-d]pyrimidin-4(3H)-one;
3-(4-chlorophenyl)-2-(2-[4-trifluoromethylphenyl]ethylthio)pyrido[3,2-d]pyrimidin-4(3H)-one;
6-(4-chlorophenyl)-5-[3-fluorobenzylthio][1,3]thiazolo[4,5-d]pyrimidin-7(6H)-one;
6-(4-chlorophenyl)-5-{2-(4-chlorophenyl)ethylthio}[1,3]thiazolo[4,5-d]pyrimidin-7(6H)-one;
2-{6-chloro-1-benzothien-3-ylmethylthio}-1-(4-chlorophenyl)-9-methyl-1,9-dihydro-6H-purin-6-one;
2-{5-chloro-1-benzothien-3-ylmethylthio}-1-(4-chlorophenyl)-9-ethyl-1,9-dihydro-6H-purin-6-one;
2-{5-chloro-1-benzothien-3-ylmethylthio}-1-(4-chlorophenyl)-9-isopropyl-1,9-dihydro-6H-purin-6-one;
3-(6-chloropyridin-3-yl)-2-[3-fluorobenzylthio]thieno[3,2-d]pyrimidin-4(3H)-one;
2-{5-chloro-1-benzothien-3-ylmethylthio}-3-[4-trifluoromethylphenyl]pyrido[3,2-d]pyrimidin-4(3H)-one;
1-(3-chlorophenyl)-9-methyl-2-(2-[4-trifluoromethylphenyl]-ethylthio)-1,9-dihydro-6H-purin-6-one;
1-(4-chlorophenyl)-2-[3,4-dichlorobenzylthio]-9-methyl-1,9-dihydro-6H-purin-6-one;
1-(4-chlorophenyl)-9-cyclopropyl-2-(2-[4-trifluoromethylphenyl]ethylthio)-1,9-dihydro-6H-purin-6-one;
1-(4-chlorophenyl)-9-ethyl-2-(2-[4-trifluoromethylphenyl]ethylthio)-1,9-dihydro-6H-purin-6-one;
3-[4-trifluoromethylphenyl]-2-(2-[4-trifluoromethylphenyl]ethylthio)thieno[3,2-d]pyrimidin-4(3H)-one;
3-[4-trifluoromethylphenyl]-2-(2-[4-trifluoromethylphenyl]ethylthio)pyrido[3,2-d]pyrimidin-4(3H)-one;
1-(4-chlorophenyl)-2-{2-(2,4-dichlorophenyl)ethylthio}-9-methyl-1,9-dihydro-6H-purin-6-one;
3-(4-chlorophenyl)-2-[3,4-dichlorobenzylthio]pyrido[3,2-d]pyrimidin-4(3H)-one;
2-[3-chloro-4-fluorobenzylthio]-3-(4-chlorophenyl)pyrido[3,2-d]pyrimidin-4(3H)-one;
1-(4-chlorophenyl)-2-(2-[3-fluoro-4-trifluoromethylphenyl]ethylthio)-9-methyl-1,9-dihydro-6H-purin-6-one;
3-(4-fluorophenyl)-2-(2-[4-trifluoromethylphenyl]ethylthio)pyrido[3,2-d]pyrimidin-4(3H)-one;
1-(4-fluorophenyl)-9-methyl-2-(2-[4-trifluoromethylphenyl]ethylthio)-1,9-dihydro-6H-purin-6-one;
1-(4-chlorophenyl)-9-methyl-2-(2-[4-trifluoromethoxyphenyl]ethylthio)-1,9-dihydro-6H-purin-6-one;
1-(4-chlorophenyl)-2-{2-(4-fluorophenyl)ethylthio}-9-methyl-1,9-dihydro-6H-purin-6-one;
1-(4-chlorophenyl)-2-[2,4-dichlorobenzylthio]-9-methyl-1,9-dihydro-6H-purin-6-one;
2-{5-chloro-1-benzothien-3-ylmethylthio}-9-methyl-1-[4-trifluoromethylpheny]-1,9-dihydro-6H-purin-6-one;
9-methyl-1-[4-trifluoromethylphenyl]-2-(2-[4-trifluoromethylphenyl]ethylthio)-1,9-dihydro-6H-purin-6-one;
4-[2-{5-chloro-1-benzothien-3-ylmethylthio}-4-oxopyrido[3,2-d]pyrimidin-3(4H)-yl]benzonitrile;
ethyl {1-(4-chlorophenyl)-9-methyl-6-oxo-6,9-dihydro-1H-purin-2-ylthio}acetate;
4-[4-oxo-2-(2-[4-trifluoromethylphenyl]ethylthio)pyrido[3,2-d]pyrimidin-3(4H)-yl]benzonitrile;
1-(4-chlorophenyl)-2-[3,4-dichlorobenzylthio]-9-ethyl-1,9-dihydro-6H-purin-6-one;
1-(4-chlorophenyl)-9-propyl-2-(2-[4-trifluoromethylphenyl]ethylthio)-1,9-dihydro-6H-purin-6-one;
1-(4-chlorophenyl)-9-methyl-2-(2-[4-trifluoromethylphenyl]ethylamino)-1,9-dihydro-6H-purin-6-one;
1-(4-chlorophenyl)-2-[3,4-dichlorobenzylthio]9-propyl-1,9-dihydro-6H-purin-6-one;
2-{5-chloro-1-benzothien-3-ylmethylthio}-9-methyl-1-[4-trifluoromethoxyphenyl]-1,9-dihydro-6H-purin-6-one;
2-{5-chloro-1-benzothien-3-ylmethylthio}-1-(4-chlorophenyl)-9-(cyclopropylmethyl)-1,9-dihydro-6H-purin-6-one;
1-(4-chlorophenyl)-2-{2-(4-chlorophenyl)-2-oxoethylthio}-9-(cyclopropylmethyl)-1,9-dihydro-6H-purin-6-one;
1-(4-chlorophenyl)-9-cyclopropyl-2-[3-fluorobenzylthio]-1,9-dihydro-6H-purin-6-one;
2-[3-chloro-4-fluorobenzylthio]-1-(4-chlorophenyl)-9-cyclopropyl-1,9-dihydro-6H-purin-6-one;
1-(4-chlorophenyl)-9-cyclopropyl-2-[3,4-dichlorobenzylthio]-1,9-dihydro-6H-purin-6-one;
1-(4-chlorophenyl)-9-cyclopropyl-2-{3-trifluoromethylbenzylthio}-1,9-dihydro-6H-purin-6-one;
2-[3-chlorobenzylthiol-1-(4-chlorophenyl)-9-cyclopropyl-1,9-dihydro-6H-purin-6-one;
2-{5-chloro-1-benzothien-3-ylmethylthio}-1-(4-chlorophenyl)-9-cyclopropyl-1,9-dihydro-6H-purin-6-one;
1-(4-chlorophenyl)-2-{2-(4-chlorophenyl)-2-oxoethylthio}-9-cyclopropyl-1,9-dihydro-6H-purin-6-one;
1-(4-chlorophenyl)-9-cyclopropyl-2-(2-oxo-2-[4-trifluoromethylphenyl]ethylthio)-1,9-dihydro-6H-purin-6-one;
1-(4-chlorophenyl)-9-cyclopropyl-2-{2-(4-fluorophenyl)-2-oxoethylthio}-1,9-dihydro-6H-purin-6-one;
1-(4-chlorophenyl)-9-cyclopropyl-2-[2,4-dichlorobenzylthio]-1,9-dihydro-6H-purin-6-one;
1-(4-chlorophenyl)-9-cyclopropyl-2-{2-(2,4-dichlorophenyl)ethylthio}-1,9-dihydro-6H-purin-6-one;
3-(4-chlorophenyl)-2-[3-fluorobenzylthio]-7-methylthieno[3,2-d]pyrimidin-4(3H)-one;
2-{5-chloro-1-benzothien-3-ylmethylthio)}-3-(4-chlorophenyl)-7-methylthieno[3,2-d]pyrimidin-4(3H)-one;
4-2-[3-fluorobenzylthio]-4-oxopyrido[3,2-d]pyrimidin-3(4H)-yl]benzonitrile;
1-(4-chlorophenyl)-9-cyclopropyl-2-(2-[2-hydroxy-4-trifluoromethylphenyl]ethylthio)-1,9-dihydro-6H-purin-6-one;
2-{5-chloro-1-benzothien-3-ylmethylthio}-9-ethyl-1-(4-methylphenyl)-1,9-dihydro-6H-purin-6-one;
2-{5-chloro-1-benzothien-3-ylmethylthio}-1-(4-chlorophenyl)-9-propyl-1,9-dihydro-6H-purin-6-one;
1-(4-chlorophenyl)-9-cyclopropyl-2-(2-[2-fluoro-4-trifluoromethylphenyl]ethylthio)-1,9-dihydro-6H-purin-6-one;
1-(4-bromophenyl)-2-{5-chloro-1-benzothien-3-ylmethylthio}-9-ethyl-1,9-dihydro-6H-purin-6-one;
2-[1,3-benzothiazol-2-ylmethylthio]-1-(4-chlorophenyl)-9-cyclopropyl-1,9-dihydro-6H-purin-6-one;
1-(4-chlorophenyl)-9-cyclopropyl-2-{2-fluoro-4-trifluoromethylbenzylthio}-1,9-dihydro-6H-purin-6-one;
1-(4-chlorophenyl1-9-cyclopropyl-2-{2-fluoro-5-trifluoromethylbenzylthio)-1,9-dihydro-6H-purin-6-one;
1-(4-chlorophenyl)-9-cyclopropyl-2-{3-fluoro-4-trifluoromethylbenzylthio}-1,9-dihydro-6H-purin-6-one;
1-(4-chlorophenyl)-9-cyclopropyl-2-(5-trifluoromethyl-1,3-benzothiazol-2-ylmethylthio)-1,9-dihydro-6H-purin-6-one;
2-{5-chloro-1-benzothien-3-ylmethylthio}-1-[4-dimethylaminophenyl]-9-ethyl-1,9-dihydro-6H-purin-6-one;
3-(4-chlorophenyl)-2-[3,4-dichlorobenzylthio]-7-methylthio[3,2-d]pyrimidin-4(3H)-one;
2-{2-(2,4-dichlorophenyl)ethylthio}-1-(4-fluorophenyl-9-methyl-1,9-dihydro-6H-purin-6-one;
1-(4-chlorophenyl)-2-{2-(2,4-dichlorophenyl)ethylthio}-9-ethyl-1,9-dihydro-6H-purin-6-one;
1-(4-chlorophenyl)-9-ethyl-2-pentylthio-1,9-dihydro-6H-purin-6-one;
1-(4-chlorophenyl)-9-ethyl-2-[3-methylbutylthio]-1,9-dihydro-6H-purin-6-one;
1-(4-chlorophenyl)-2-[2-cyclohexylethylamino]-9-ethyl-1,9-dihydro-6H-purin-6-one;
1-(4-chlorophenyl)-2-(2-[2-chloro-4-trifluoromethylphenyl]ethylthio)-9-methyl-1,9-dihydro-6H-purin-6-one;
1-(4-chlorophenyl)-2-(2-[2-chloro-4-trifluoromethylphenyl]ethylthio)-9-ethyl-1,9-dihydro-6H-purin-6-one;
2-{2-(2,4-dichlorophenyl)ethylthio}-9-ethyl-1-(4-fluorophenyl)-1,9-dihydro-6H-purin-6-one;
9-ethyl-1-(4-fluorophenyl)-2-(2-[2-fluoro-4-trifluoromethylphenyl]ethylthio)-1,9-dihydro-6H-purin-6-one;
2-(2-[2-chloro-4-trifluoromethylphenyl]ethylthio)-9-ethyl-1-(4-fluorophenyl)-1,9-dihydro-6H-purin-6-one;
2-{5-chloro-1-benzothien-3-ylmethylamino}-1-(4-chlorophenyl)-9-ethyl-1,9-dihydro-6H-purin-6-one;
1-(4-chlorophenyl)-9-ethyl-2-[3-fluorobenzylamino]-1,9-dihydro-6H-purin-6-one;
2-{5-chloro-1,3-benzothiazol-2-ylmethylthio}-1-(4-chlorophenyl)-9-ethyl-1,9-dihydro-6H-purin-6-one;
1-(2,4-dichlorophenyl)-2-{2-(2,4-dichlorophenyl)ethylthio}-9-methyl-1,9-dihydro-6H-purin-6-one;
1-(4-chlorophenyl)-2-(2-[2-fluoro-4-trifluoromethylphenyl]ethylthio)-9-methyl-1,9-dihydro-6H-purin-6-one;
1-(4-chlorophenyl)-9-ethyl-2-(2-[2-fluoro-4-trifluoromethylphenyl]ethylthio)-1,9-dihydro-6H-purin-6-one;
1-(4-chlorophenyl)-2-{2-(2,4-dichlorophenyl)-2-oxoethylthio}-9-methyl-1,9-dihydro-6H-purin-6-one;
9-ethyl-1-(4-fluorophenyl)-2-[3,3,3-trifluoropropylthio]-1,9-dihydro-6H-purin-6-one;
1-(4-chlorophenyl)-9-ethyl-2-[3,3,3-trifluoropropylamino]1,9-dihydro-6H-purin-6-one;
9-cyclopropyl-2-{2-(2,4-dichlorophenyl)ethylthio}-1-(4-fluorophenyl)-1,9-dihydro-6H-purin-6-one;
1-(4-chlorophenyl)-2-{2-(2,4-dichlorophenyl)ethylthio}-9-(2-hydroxyethyl)-1,9-dihydro-6H-purin-6-one;
9-cyclopropyl-1-(4-fluorophenyl)-2-(3,3,3-trifluoropropylthio)-1,9-dihydro-6H-purin-6-one;
1-(4-chlorophenyl)-2-{2-(2,4-dichlorophenyl)ethylamino}-9-ethyl-1,9-dihydro-6H-purin-6-one;
1-(4-chlorophenyl)-2-[cyclobutylmethylthio]-9-ethyl-1,9-dihydro-6H-purin-6-one;
9-cyclopropyl-1-(4-fluorophenyl)-2-(2-[2-fluoro-4-trifluoromethylphenyl]ethylthio)-1,9-dihydro-6H-purin-6-one;
1-(4-chlorophenyl)-9-ethyl-2-[3,3,3-trifluoro-2-hydroxypropylthio]-1,9-dihydro-6H-purin-6-one;
1-(4-chlorophenyl)-9-ethyl-2-[3,3,3-trifluoro-2,2-dihydroxypropylthiol]-1,9-dihydro-6H-purin-6-one;
1-(4-chlorophenyl)-2-[2-cyclopentylethylthio]-9-ethyl-1,9-dihydro-6H-purin-6-one;
1-(4-chlorophenyl)-9-ethyl-2-{2-methyl-1,3-thiazol-4-ylmethylthio}-1,9-dihydro-6H-purin-6-one;
1-(4-chlorophenyl)-9-methyl-2-[4,4,4-trifluorobutylthio]-1,9-dihydro-6H-purin-6-one;
1-(4-chlorophenyl)-2-(2-[2-fluoro-4-trifluoromethylphenyl]ethylamino)-9-methyl-1,9-dihydro-6H-purin-6-one;
1-(4-chlorophenyl)-2-[cyclopentylmethylthio]-9-ethyl-1,9-dihydro-6H-purin-6-one;
1-(4-chlorophenyl)-9-methyl-2-[4,4,4-trifluorobutylamino]-1,9-dihydro-6H-purin-6-one;
3-(4-chlorophenyl)-2-[3,3,3-trifluoropropylthio]pyrido[3,2-d]pyrimidin-4(3H)-one;
4-{9-methyl-6-oxo-2-[4,4,4-trifluorobutylthio]-6,9-dihydro-1H-purin-1-yl}benzonitrile;
4-{9-ethyl-6-oxo-2-[3,3,3-trifluoropropylthio]-6,9-dihydro-1H-purin-1-yl}benzonitrile;
1-(3-chloro-4-fluorophenyl)-9-methyl-2-[4,4,4-trifluorobutylthio]-1,9-dihydro-6H-purin-6-one;
1-(3-chloro-4-fluorophenyl)-9-methyl-2-[3,3,3-trifluoropropylthio]-1,9-dihydro-6H-purin-6-one;
1-(4-chlorophenyl)-9-methyl-2-{2-methyl-1,3-thiazol-4-ylmethylthio}-1,9-dihydro-6H-purin-6-one;
1-(4-chlorophenyl)-9-propyl-2-[3,3,3-trifluoropropylthio]-1,9-dihydro-6H-purin-6-one;
1-(4-chlorophenyl)-9-propyl-2-[4,4,4-trifluorobutylthio]-1,9-dihydro-6H-purin-6-one;
1-(4-chlorophenyl)-9-isopropyl-2-[3,3,3-trifluoropropylthio]-1,9-dihydro-6H-purin-6-one;
1-(4-chlorophenyl)-9-isopropyl-2-[4,4,4-trifluorobutylthio]-1,9-dihydro-6H-purin-6-one;
1-(4-chlorophenyl)-2-[3-fluoropropylthio]-9-methyl-1,9-dihydro-6H-purin-6-one;
1-(4-chlorophenyl)-9-ethyl-2-[3-fluoropropylthio]-1,9-dihydro-6H-purin-6-one;
1-(4-chlorophenyl)-9-methyl-2-(4-trifluoromethyl-1,3-thiazol-2-ylmethylthio)-1,9-dihydro-6H-purin-6-one;
1-(4-chlorophenyl)-9-methyl-2-(6-trifluoromethylpyridin-3-ylmethylthio)-1,9-dihydro-6H-purin-6-one;
1-(4-chlorophenyl)-9-ethyl-2-(6-trifluoromethylpyridin-3-ylmethylthio)-1,9-dihydro-6H-purin-6-one;
1-(4-chlorophenyl)-9-ethyl-2-(4-trifluoromethyl-1,3-thiazol-2-ylmethylthio)-1,9-dihydro-6H-purin-6-one;
4-[9-ethyl-6-oxo-2-(4-trifluoromethyl-1,3-thiazol-2-ylmethylthio)-6,9-dihydro-1H-purin-1-yl]benzonitrile;
1-(3,4-difluorophenyl)-9-methyl-2-[3,3,3-trifluoropropylthio)-1,9-dihydro-6H-purin-6-one;
1-(3,4-difluorophenyl)-9-methyl-2-[4,4,4-trifluorobutylthio]-1,9-dihydro-6H-purin-6-one;
1-(4-chlorophenyl)-9-methyl-2-(2-trifluoromethyl-1,3-thiazol-4-ylmethylthio)-1,9-dihydro-6H-purin-6-one;
1-(3-fluoro-4-methylphenyl)-9-methyl-2-[3,3,3-trifluoropropylthio]-1,9-dihydro-6H-purin-6-one;
1-(4-chlorophenyl)-9-methyl-2-(4-methylpiperazin-1-yl)-1,9-dihydro-6H-purin-6-one;
1-(4-chloro-2-fluorophenyl)-9-methyl-2-[3,3,3-trifluoropropylthio]-1,9-dihydro-6H-purin-6-one;
1-(4-fluorophenyl)-9-methyl-2-[3,3,3-trifluoropropylthio]-1,9-dihydro-6H-purin-6-one;
1-(4-chloro-3-fluorophenyl)-9-methyl-2-[3,3,3-trifluoropropylthio]-1,9-dihydro-6H-purin-6-one;
1-(4-chloro-3-methoxyphenyl)-9-methyl-2-[3,3,3-trifluoropropylthio]-1,9-dihydro-6H-purin-6-one;
2-{1-(4-chlorophenyl)-9-ethyl-6-oxo-6,9-dihydro-1H-purin-2-ylthio}-N-methylacetamide;
2-{1-(4-chlorophenyl)-9-ethyl-6-oxo-6,9-dihydro-1H-purin-2-ylthio}-N,N-diethylacetamide;
1-(4-chlorophenyl)-9-ethyl-2-{5-methylisoxazol-3-ylmethylthio}-1,9-dihydro-6H-purin-6-one;
1-(4-chlorophenyl)-9-methyl-2-methylthio-1,9-dihydro-6H-purin-6-one;
1-(4-fluorophenyl)-9-methyl-2-(4-trifluoromethyl-1,3-thiazol-2-yl]methylthio)-1,9-dihydro-6H-purin-6-one;
9-ethyl-1-(4-fluorophenyl)-2-(4-trifluoromethyl-1,3-thiazol-2-ylmethylthio)-1,9-dihydro-6H-purin-6-one;
1-(3-bromo-4-chlorophenyl)-9-methyl-2-[3,3,3-trifluoropropylthio]-1,9-dihydro-6H-purin-6-one;
1-(4-chlorophenyl)-2-cyclohexylamino-9-ethyl-1,9-dihydro-6H-purin-6-one;
1-(4-chlorophenyl)-2-[3,3,3-trifluoropropylthio]-1,9-dihydro-6H-purin-6-one;
1-(4-chlorophenyl)-9-ethyl-2-{2-methyl-1,3-thiazol-4-ylmethylamino}-1,9-dihydro-6H-purin-6-one;
3-(4-chlorophenyl)-2-(2-trifluoromethyl-1,3-thiazol-4-ylmethylthio)pyrido[3,2-d]pyrimidin-4(3H)-one;
3-(4-chlorophenyl)-2-(2-trifluoromethyl-1,3-thiazol-4-ylmethylamino)pyrido[3,2-d]pyrimidin-4(3H)-one;
1-(4-chloro-3-ethoxyphenyl)-9-ethyl-2-[3,3,3-trifluoropropylthio]-1,9-dihydro-6H-purin-6-one;
1-(4-chloro-3-isopropoxyphenyl)-9-ethyl-2-[3,3,3-trifluoropropylthio]-1,9-dihydro-6H-purin-6-one;
1-(4-chlorophenyl)-9-ethyl-2-{4-[3-trifluoromethylpyridin-2-yl]piperazin-1-yl}-1,9 -dihydro-6H-purin-6-one;
3-(4-fluorophenyl)-2-(2-trifluoromethyl-1,3-thiazol-4-ylmethyl}thio)pyrido[3,2-d]pyrimidin-4(3H)-one;
6-(4-chlorophenyl)-5-(2-trifluoromethyl-1,3-thiazol-4-ylmethylthio)[1,3]thiazolo[5,4-d]pyrimidin-7(6H)-one;
3-(4-chlorophenyl)-2-cyclohexylaminopyrido[3,2-d]pyrimidin-4(3H)-one;
3-(4-chlorophenyl)-2-[3,3,3-trifluoropropylthio]thieno[3,2-d]pyrimidin-4(3H)-one;
1-(4-chlorophenyl)-9-ethyl-2-(2-trifluoromethyl-1,3-thiazol-4-ylmethylamino)-1,9-dihydro-6H-purin-6-one;
9-ethyl-1-(4-fluorophenyl)-2-(2-trifluoromethyl-1,3-thiazol-4-ylmethylthio)-1,9-dihydro-6H-purin-6-one;
3-(4-chlorophenyl)-7-methyl-2-[3,3,3-trifluoropropylthio]thieno[3,2-d]pyrimidin-4(3H)-one;
3-(4-chlorophenyl)-7-methyl-2-(2-[4-trifluoromethylphenyl]ethylamino)thieno[3,2-d]pyrimidin-4(3H)-one;
3-(4-chlorophenyl)-7-methyl-2-[3,3,3-trifluoropropylamino]thieno[3,2-d]pyrimidin-4(3H)-one;
1-(4-chlorophenyl)-2-{2-(2,4-dichlorophenyl)ethylthio}-9-[2-dimethylaminoethyl]-1,9-dihydro-6H-purin-6-one;
1-(4-chlorophenyl)-9-cyclopropyl-2-(2-[2-methoxy-4-trifluoromethylphenyl]ethylthio)-1,9-dihydro-6H-purin-6-one;
1-(4-chlorophenyl)-9-ethyl-2-[methyl(3,3,3-trifluoropropyl)amino]-1,9-dihydro-6H-purin-6-one;
2-[[5-chloro-1-benzothien-3-ylmethyl](methyl)amino]-1-(4-chlorophenyl)-9-ethyl-1,9-dihydro-6H-purin-6-one; and
2-chloro-5-{9-methyl-6-oxo-2-[3,3,3-trifluoropropylthio]-6,9-dihydro-1H-purin-1-yl}benzonitrile;
or a pharmaceutically acceptable salt thereof.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

As used herein, the terms “haloC₁₋₆alkyl” and “haloC₁₋₆alkoxy” means a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂F, CHF₂, CH₂CH₂F, CH₂CHF₂, CH₂CF₃, OCF₃, OCH₂CH₂F, OCH₂CHF₂ or OCH₂CF₃, and most especially CF₃ and OCF₃.

The cycloalkyl groups referred to herein may represent, for example, cyclopropyl, cyclobutyl, cyclopentyl or cyclohexyl. Such groups also include, for example, cyclopropylmethyl and cyclohexylmethyl.

As used herein, the terms “alkenyl” and “alkynyl” as a group or part of a group means that the group is straight or branched. Examples of suitable alkenyl groups include vinyl and allyl. A suitable alkynyl group is acetylene or propargyl.

When used herein, the term “halogen” means fluorine, chlorine, bromine and iodine. The most preferred halogens are fluorine and chlorine, especially chlorine.

Examples of 6-membered saturated rings are morpholine, piperidine and piperazine.

Examples of 6-membered heteroaromatic rings are pyridine, pyrimidine, pyrazine, pyridazine and triazine.

Examples of 5-membered heteroaromatic rings are thiophene, furan, pyrrole, imidazole, pyrazole, oxazole, isoxazole, thiazole, isothiazole, 1,2,3-triazole, 1,2,4-triazole, oxadiazole, thiadiazole and tetrazole.

Examples of 9- or 10-membered fused bicyclic heteroaromatic rings include benzofuran, benzothiophene, benzimidazole, benzoxazole, benzothiazole, benzisothiazole, quinoline, isoquinoline and cinnoline.

In a further aspect of the present invention, the compounds of formula I may be prepared in the form of a pharmaceutically acceptable salt, especially an acid addition salt.

For use in medicine, the salts of the compounds of formula I will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. A further salt is the acid addition salt with benzenesulfonic acid. Preferred pharmaceutically acceptable salts of the compounds of the present invention are the besylate salts. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the compound of formula I with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention also includes within its scope N-oxides of the compounds of formula I above. In general, such N-oxides may be formed on any available nitrogen atom. The N-oxides may be formed by conventional means, such as reacting the compound of formula I with oxone in the presence of wet alumina.

The present invention includes within its scope prodrugs of the compounds of formula I above. In general, such prodrugs will be functional derivatives of the compounds of formula I which are readily convertible in vivo into the required compound of formula I. Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula I and salts thereof, for example, hydrates.

The compounds according to the invention may have one or more asymmetric centres, and may accordingly exist both as enantiomers and as diastereoisomers. It is to be understood that all such isomers and mixtures thereof are encompassed within the scope of the present invention. Furthermore, the compounds of formula I may also exist in tautomeric forms and the invention includes within its scope both mixtures and separate individual tautomers.

The compounds may exist in different isomeric forms, all of which are encompassed by the present invention.

The present invention further provides pharmaceutical compositions comprising one or more compounds of formula I in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 50, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form. of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of painful conditions such as those listed below, a suitable dosage level is about 1.0 mg to 15 g per day, preferably about 5.0 mg to 1 g per day, more preferably about 5 mg to 500 mg per day, especially 10 mg to 100 mg per day. The compounds may be administered on a regimen of 1 to 4 times per day.

It will be appreciated that the amount of a compound of formula I required for use in any treatment will vary not only with the particular compounds or composition selected but also with the route of administration, the nature of the condition being treated, and the age and condition of the patient, and will ultimately be at the discretion of the attendant physician.

The invention further provides a compound of formula I as defined above, or a pharmaceutically acceptable salt thereof, for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical-pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain, chronic pelvic pain, chronic prostatitis and endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis, asthma and rhinitis, including allergic rhinitis such as seasonal and perennial rhinitis, and non-allergic rhinitis and cough; autoimmune diseases; and immunodeficiency disorders. The compounds of the present invention may also be used to treat depression. They may also be used to treat gastro-oesophageal reflux disease (GERD), particularly the pain associated with GERD.

Thus, according to a further aspect, the present invention provides a compound of formula I for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula I or a composition comprising a compound of formula I.

According to a further or alternative aspect, the present invention provides a compound of formula I for use in the manufacture of a medicament for the treatment or prevention of a disease or condition in which pain and/or inflammation predominates.

The present invention also provides a method for the treatment or prevention of a disease or condition in which pain and/or inflammation predominates, which method comprises administration to a patient in need thereof of an effective amount of a compound of formula I or a composition comprising a compound of formula I.

According to a further aspect of the present invention, it may be desirable to treat any of the aforementioned conditions with a combination of a compound according to the present invention and one or more other pharmacologically active agents suitable for the treatment of the specific condition. The compound of formula I and the other pharmacologically active agent(s) may be administered to a patient simultaneously, sequentially or in combination.

Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR2B antagonists, bradykinin antagonists, anti-migraine agents, anticonvulsants such as oxcarbazepine and carbamazepine, antidepressants (such as TCAs, SSRIs, SNRIs, substance P antagonists, etc.), spinal blocks, gabapentin, pregabalin and asthma treatments (such as θ₂-adrenergic receptor agonists or leukotriene D₄ antagonists (e.g. montelukast).

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib and tilicoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or a pharmaceutically acceptable salt thereof. Suitable anti-migraine agents of use in conjunction with a compound of the present invention include CGRP-antagonists, ergotamines or 5-HT₁ agonists, especially sumatriptan, naratriptan, zolmatriptan or rizatriptan.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

Compounds of formula I in which X is S can be made by reacting a compound of formula II with a compound of formula III: 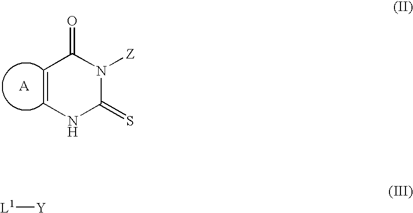
wherein A, Y and Z are as defined above and L¹ is a leaving group such as Cl, Br, or I. The reaction is generally carried out in the presence of a mild base, such as potassium carbonate, in a solvent such as acetonitrile from room temperature to 75° C. for two to 24 hours.

Compounds of formula II can be made by reacting a compound of formula IV with a compound of formula V: 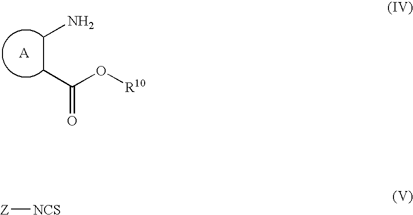
wherein A and Z are as defined above and R¹⁰ is a C₁₋₆alkyl group such as methyl. The reaction is generally carried out in a solvent such as acetonitrile, ethanol or pyridine from 45° C. to reflux for from 2 to 24 hours. A catalytic amount of a compound such as 4-dimethylaminopyridine is generally added. If necessary the reaction-completing ring closure is effected by the addition of a base such as potassium hydroxide or sodium hydroxide in a solvent such as methanol, water or tetrahydrofuran for from 30 minutes to 3 hours from room temperature to reflux. If necessary, the product is acidified using an acid such as HCl to produce a salt.

The compound of formula IV can be made by reacting a compound of formula VI with an alcohol of formula VII: 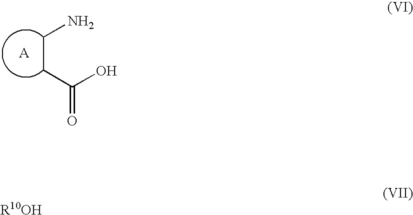
wherein A and R¹⁰ are as defined above, generally in the presence of an acid, such as sulphuric acid, at about 80° C. for from 3 to 7 days.

The compound of formula VI can be made by reacting a compound of formula VIII: 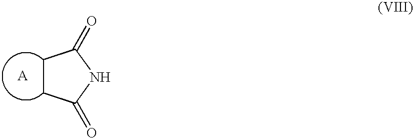
wherein A is as defined above, with an oxidizing agent such as sodium hypobromite (which can be prepared by reacting bromine with 10% NaOH_{(aq)} at about 0° C.). The reaction is generally carried out at about 80° C. for about 45 minutes.

The compound of formula IV can alternatively be prepared by reacting a compound of formula IX: 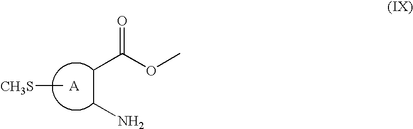
wherein A is as defined above with a hydrogenating agent such as Raney Nickel in the presence of hydrogen at about 45 psi for about 1 week generally in a solvent such as ethanol/water mixture.

Alternatively the compound of formula IV can be made by reacting a compound of formula X: 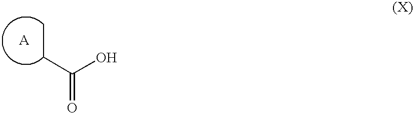
wherein A is as defined above firstly with a nitrating agent such as ammonium nitrate generally in the presence of an acid such as sulphuric acid at about 100° C. for about 2 days, secondly with a compound of formula VII under the conditions described for reaction with the compound of formula VI and thirdly under hydrogenating conditions such as hydrogen on 10% Pd/C in a solvent mixture of ethanol and water for about 4 hours.

Compounds of formula I in which X is NR¹, where R¹ is as defined above, can be made by reacting a compound of formula XI with a compound of formula XII: 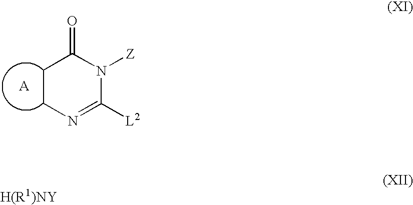
wherein A, R¹, Y and Z are as defined above and L² is a leaving group such as chlorine. The reaction is generally carried out in a solvent such as acetonitrile in the presence of a base such as potassium carbonate at about reflux for four or five hours.

Compounds of formula XI can be made by reacting a compound of formula II with a chlorinating agent such as PCl₅ in POCl₃ or POCl₃ at about 110° C. for 36 hours or in the presence of pyridine at about 100° C. or reflux for 6 to 24 hours. They can also be made under the same conditions starting with a compound of formula XIII: 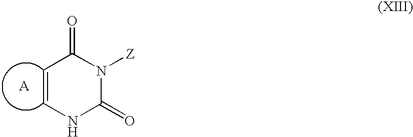
wherein A and Z are as defined above.

Compounds of formula XIII can be made in the same way as compounds of formula II but using a compound of formula XIV:
Z—NCO (XIV)
wherein Z is as defined above generally in a solvent such as ethyl acetate at about reflux for about 8 hours, followed by a ring closure as described for the preparation of compounds of formula II.

Compounds of formula XII can be made by reacting a compound of formula XV:
Y¹CN (XV)
wherein Y¹ is (CR²R³)ₙ₋₁(CO)ₚ(NR⁴)_{q}W and n is 1, 2, 3 or 4, with sodium trifluoroacetoxyborohydride in a solvent such as tetrahydrofuran.

Compounds of formula I in which X is O can be prepared by reacting a compound of formula XI with a compound of formula XVI:
HOY (XVI)
wherein Y is as defined above. The reaction is generally carried out in the presence of a strong base such as sodium hydride in a solvent such as tetrahydrofuran from about 0° C. to room temperature for about 18 hours.

The compound of formula II can alternatively be prepared by reacting a compound of formula XVII: 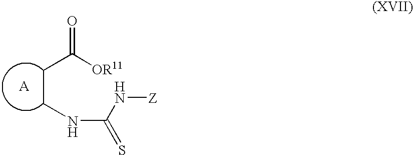
wherein A and Z are as defined above and R¹¹ is a C₁₋₆alkyl group such as ethyl or methyl, with a base such as potassium hydroxide or sodium hydroxide, generally in a solvent such as water at about 80° C.

The compound of formula XVII can be prepared by reacting a compound of formula XVIII with a compound of formula XIX: 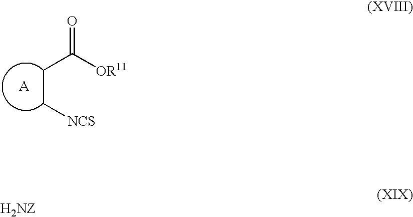
wherein A, R¹¹ and Z are as defined above, generally in a solvent such as methylcyanide at about 40 to 80° C. A catalyst such as dimethylaminopyridine (DMAP) may be used.

The compound of formula XVIII can be prepared by reacting a compound of formula XX: 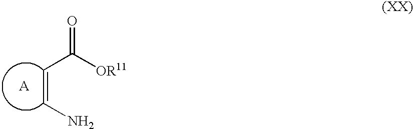
wherein A and R¹¹ are as defmed above with a thiocarbonyl transfer reagent such as 1,1′-thiocarbonyldi-2(1H)-pyridone (TDP), generally in a solvent such as dichloromethane at room temperature.

The compounds of formula III can be prepared by reacting a compound of formula XX with a compound of formula XXII: 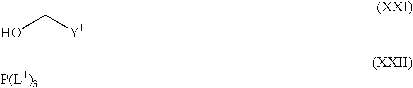
wherein Y¹ and L¹ are as defined above. The reaction is generally carried out at room temperature followed by heating to about 100° C. for around 2 hours.

Compounds of formula X=can be made by reacting a compound of formula XXII with a reducing agent such as borane dimethylsulfide: 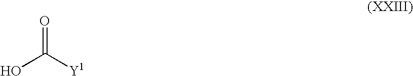
wherein Y¹ is as defined above. The reaction is generally carried out in a solvent such as tetrahydrofuran at room temperature followed by the addition of a strong base such as sodium hydroxide or potassium hydroxide.

Compounds of formula XXI may alternatively be prepared by perfusing a compound of formula XXIV with oxygen and ozone: 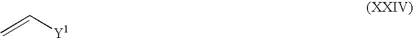
wherein Y¹ is as defined above, generally in solvents such as dichloromethane and methanol at about −78° C. A reducing agent such as sodium borohydride may subsequently be added.

The compounds of formula III can alternatively be made by reacting a compound of formula XXI with L³₂ wherein L³ is a halogen group such as iodine or bromine. The reaction is generally carried out in the presence of triphenylphosphine, in a solvent such as dichloromethane or dimethylforamide at about 0° C.

Compounds of formula XX may alternatively be made by reacting a compound of formula XXV with a reducing agent such as diisobutyl aluminium hydride: 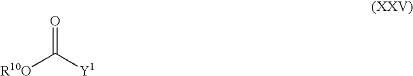
wherein R¹⁰ and Y¹ are as defined above, generally in a solvents such as tetrahydrofuran at about −78° C. A further amount of reducing agent may subsequently be added to the reaction mixture, followed by the addition of an alcohol such as methanol.

Compounds of formula XII wherein R¹ is hydrogen can be made by reacting a compound of formula XXVI: 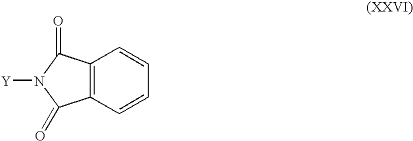
wherein Y is as defined above with hydrazine hydrate, generally in solvents such as tetrahydrofuran and ethanol at room temperature.

Where the synthesis of intermediates and starting materials is not described these compounds are commercially available or can be made from commercially available compounds by standard methods, or by extension from the Descriptions and Examples herein.

Compounds of formula I may be converted to other compounds of formula I by known methods or by methods described in the Descriptions and Examples.

Thus, compounds of formula I wherein A is substituted by hydroxyC₁₋₆alkyl may be converted to compounds wherein A is substituted by an amino moiety by reacting with a mixture of triphenylphosphine and L³₂ wherein L³ is a halogen group such as iodine, generally in a solvent such as dichloromethane at room temperature. An amine such as dimethylamine can subsequently be added at room temperature to produce the desired compound.

Similarly, compounds of formula I wherein X is NR¹ and R¹ is hydrogen may be converted to compounds of formula I wherein R¹ is C₁₋₆alkyl by reacting with an alkylating agent such as sodium hydride, generally in a solvent such as dimethylformide, followed by the addition of C₁₋₆alkyl-L¹ wherein L¹ is as defined above.

The halogen substituent such as bromine on a compound of formula I may be converted to a cyano group by reacting with zinc cyanide. The reaction may be carried out in the presence of zinc dust and a catalyst such as [1,1′-bis(diphenylphosphino)ferrocene]dichloropalladium(II).

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P.G.M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention.

### Description 1 3-Aminoisonicotinic acid

Bromine (3.5 ml, 69.0 mmol) was added to a solution of 10% aqueous sodium hydroxide (120 ml) at 0° C. to give a pale yellow solution. To this solution was added 3,4-pyridinedicarboximide (10 g, 67.5 mmol) and the reaction was heated at 80° C. for 45 min. The reaction was cooled in a water bath and acidified by the addition of acetic acid (12.5 ml) causing precipitation. The solid was collected, rinsed with water (50 ml), then MeOH (50 ml) and dried to give the title compound as a beige solid (6.28 g, 67%). ¹H NMR (360 MHz, DMSO) δ 8.06 (1H, s), 7.60 (1H, d, J5.1), 7.34 (1H, d, J5.1), 3.19 (2H, brs). M/z (ES⁺) 139 (M+H⁺).

### Description 2 Methyl-3-aminoisonicotinate

Description 1 (3.55 g, 25.7 mmol), H₂SO₄ (2.5 ml) and methanol (50 ml) were heated at 80° C. for 3 days. The methanol was evaporated, the residue diluted with water (75 ml) and heated to 80° C. Solid sodium carbonate was added until effervescence ceased. The mixture was cooled and extracted with dichloromethane (2×100 ml). The combined organic layers were dried over MgSO₄ and concentrated to give the title compound as a beige solid (2.36 g, 60%). ¹H NMR (500 MHz, DMSO) δ 8.24 (1H, s), 7.74 (1H, d, J5.3), 7.46 (1 H, d, J5.2), 6.67 (2H, brs), 3.83 (3H, s). M/z (ES+) 153 (M+H⁺).

### Description 3 3-(4-Chlorolphenyl)-2-thioxo-2,3-dihydropyrido[3,4-d]pyrimidin-4(1H)-one

Description 2 (1.86 g, 12.2 mmol) and 4-chlorophenyl isothiocyanate (2.28 g, 13.5 mmol) were heated at 70° C. in acetonitrile (30 ml) with a catalytic amount of 4-dimethylaminopyridine for 24 h. The reaction was cooled and the solid product collected by filtration, washed with ether (20 ml) then dichloromethane (10 ml) and dried to give the title compound as a white solid (2.15 g, 61%). ¹H NMR (500 MHz, DMSO) δ 8 13.26 (1H, s), 8.79 (1H, s), 8.50 (1H, d, J5.1), 7.79 (1H, d, J5.2), 7.53 (2H, d, J8.6), 7.31 (2H, d, J8.6). M/z (ES⁺) 290 (M+H⁺).

### Description 4 Methyl 3-aminopyridine-2-carboxylate

A solution of 3-aminopyridine-2-carboxylic acid (Bioorg Med. Chem. 2001, 9, 2061) (1.0 g, 7.25 mmol) and H₂SO₄ (2.75 ml) in methanol (15 ml) was heated at 80° C. for 7 days. The reaction was cooled and the methanol removed by evaporation. The residue was poured into water (ca. 30 ml) and solid sodium carbonate was added until effervescence ceased (pH ˜7). The mixture was extracted with dichloromethane (4×50 ml) and the combined organic fractions dried over MgSO₄ and concentrated to give the title compound as a beige solid (0.84 g, 76%). ¹H NMR (360 MHz, CDCl₃) δ 8.07 (1H, dd, J4.2, 1.4), 7.22 (1H, dd, J8.4, 4.2), 7.05 (1H, dd, J8.4, 1.4), 5.73 (2H, brs), 3.98 (3H, s). M/z (ES⁺) 153 (M+H⁺).

### Description 5 3-(4-Chlorophenyl)-2-thioxo-2,3-dihydropyrido[3,2-d]pyrimidin-4(1l)-one

A solution of 4-chlorophenyl isothiocyanate (1.10 g, 6.48 mmol) and ethyl 3-aminopyridine-2-carboxylate (J. Chem. Soc. 1956, 1045) (1.07 g, 6.48 mmol) in acetonitrile (30 ml) was heated at reflux for 2 h, then cooled to room temperature. The solid was collected by filtration, washed with cold acetonitrile (5 ml) and dried to give the title compound as a white crystalline solid (84 mg, 4.5%). The filtrate was re-heated to reflux for 18 h and then cooled to room temperature to give a second crop of crystals. The crystals were collected by filtration, washed with acetonitrile (5 ml) and dried to give the title compound (350 mg, 19%). ¹H NMR (400 MHz, DMSO) δ 13.09 (1H, br. s), 8.60 (1H, dd, J4.3, 1.5), 7.82 (1H, dd, J8.4, 1.5), 7.77 (1H, dtd, J8.4, 4.3), 7.55 (2H, d, J8.0), 7.35 (2H, d, J8.0). M/z (ES⁺⁾ 290, 292 (M+H⁺).

### Description 6 2-Chloro-3-(4-chlorophenyl)pyrido[3.2-d]pyrimidin-4(3)-one

A solution of Description 5 (123 mg, 0.43 mmol) and phosphorous pentachloride (134 mg, 0.65 mmol) in phosphorous oxychloride (1 ml) was stirred at 100° C. for 24 h. The reaction mixture was cooled, evaporated in vacuo, and azeotroped twice with toluene. The resulting oil was then dissolved in ethyl acetate (15 ml) and washed with water (5×15 ml). The organic layer was dried over MgSO₄, filtered and evaporated to give a brown solid. The solid was dry loaded in acetonitrile onto silica and purified by flash column chromatography [eluant:ethyl acetate/dichloromethane (1:4)] to give the title compound as pale yellow solid (58 mg, 47%). ¹H NMR (360 MHz, DMSO) δ 8.86 (1H, dd, J4.4, 1.6), 8.16 (1H, dd, J8.2, 1.6), 7.91 (1H, dd, J8.2, 4.4), 7.66 (2H, d, J8.7), 7.58 (2H, d, J8.7) M/z (ES⁺⁾ 292, 294 (M+H⁺).

### Description 7 3-(4-Chlorophenyl)-2-thioxo-2.3-dihydrothieno[2.3-d]pyrimidin-4(1H)-one

A solution of methyl 2-aminothiophene-3-carboxylate (1.0 g, 6.4 mmol) and 4-chlorophenyl isothiocyanate (1.2 g, 7.1 mmol) in ethanol (10 ml) was stirred at 100° C. for 16 h. The reaction was cooled and the solid collected by filtration, washed with ether and dried to give methyl 2-(4-chlorophenylaminocarbonothioyl amino)thiophene-3-carboxylate as a white solid (0.83 g, 40%). This solid (0.63 g, 1.93 mmol) was treated with a solution of potassium hydroxide in methanol (2 M, 8 ml) at room temperature for 40 min. The reaction was acidified with 5 M aqueous hydrochloric acid leading to a thick white precipitate. The slurry was diluted with water (25 ml) to dissolve salts and then filtered. The product was washed with water and dried to give the title compound as a white solid (0.45 g, 79%). ¹H NMR (360 MHz, DMSO) δ 13.82 (1H, s), 7.53 (2H, m), 7.31 (3H, m), 7.24 (1H, d, J5.6). M/z (ES⁺⁾ 295, 297 (M+H⁺).

### Description 8 3-(4-Chlorophenyl)-2-thioxo-2,3-dihydrothieno[3,2-d]pyrimidin-4(1H)-one

A solution of methyl-3-aminothiophene-2-carboxylate (6.79 g, 4.32 mmol) and 4-chlorophenyl isothiocyanate (8.42 g, 49.6 mmol) was treated using the method of Description 7 to give methyl 3-(4-chlorophenylaminocarbonothioyl amino)thiophene-2-carboxylate (6.16 g, 44%). This solid (4.0 g, 13.6 mmol) was treated with potassium hydroxide as in Description 7 to give the title compound as a white solid (3.42 g, 96%). ¹H NMR (360 MHz, DMSO) δ 13.53 (1H, s), 8.20 (1H, d, J5.2), 7.53 (2H, d, 8.5), 7.32 (2H, d, J8.6), 7.07 (1H d, J5.2). M/z (ES⁺⁾ 295, 297 (M+H⁺).

### Description 9 3-(4-Chlorophenyl)thieno[3.2-d]pyrimidine-2,4(1H,3H)-dione

To a solution of methyl 3-aminothiophene-2-carboxylate (5,1 g, 32.5 mmol) and 4-dimethylaminopyridine (50 mg) in EtOAc (50 ml) was added 4-chlorophenyl isocyanate (5 g, 32.5 mmol)) portion-wise. After the addition was complete, the reaction was heated to reflux for 8 h. The reaction was cooled, the white solid collected by filtration and added to a solution of potassium hydroxide (3 g, 53.6 mmol) in THF/water (10:1; 35 ml). The mixture was heated to reflux for 30 min, allowed to cool, acidified with 5 M aqueous hydrochloric acid and the resultant solid collected by filtration and dried to give the title compound as a white solid (2.4 g, 26%). ¹H NMR (360 MHz, DMSO) 12.04 (1H, s), 8.12 (1H, d, J5.4), 7.53 (2H, d, J8.6), 7.36 (2H, d, J8.6), 6.99 (1H, d, J5.3).

### Description 10 2-Chloro-3-(4-chlorophenyl)thieno[3,2-d]pyrimidin-4(3H)-one

A suspension of Description 9 (2.4 g, 8.5 mmol) in phosphorus oxychloride (25 ml) and pyridine (2.5 ml) was heated to reflux for 6 h. After cooling, phosphorus oxychloride was removed in vacuo and ice-chilled water (50 ml) added. The reaction was extracted with dichloromethane (3×50 ml) and the combined organic fractions were washed with brine, dried over Na₂SO₄, and condensed to give a bright blue solid. The product was purified using a prepacked silica column, eluting with 8-25% ethyl acetate in hexane to provide a white solid (150 mg, 4%). ¹H NMR (360 MHz, CDCl₃) δ 7.87 (1H, d, J5.3), 7.53 (2H, d, J8.6), 7.34 (1H, d, J5.3), 7.24 (2H, d, J8.6).

### Description11 Ethyl 5-(4-chlorophenylaminocarbonothioylamino)-1,3-thiazole-4-carboxylate

A solution of ethyl 5-amino-1,3-thiazole-4-carboxylate (Tetrahedron 1985, 41, 5989) (544 mg, 3.16 mmol) and 4-chlorophenyl isothiocyanate (536 mg, 3.16 mmol) in acetonitrile (15 ml) was heated at reflux for 20 h. The mixture was filtered to remove insoluble material and the filtrate re-heated to reflux for a further 72 h. Flash silica (ca. 10 g) was added and the solvent evaporated. The residue was then purified by flash column chromatography [eluant: ethyl acetate/isohexane (1:3), then (1:1), then (3:1)] to give the title compound (358 mg, 33%). ¹H NMR (400 MHz, DMSO) δ 11.53 (1H, s), 11.51 (1H, s), 8.47 (1H, s), 7.58 (2H, d, J8.7), 7.47 (2H, d, J8.7), 4.35 (2H, q, J7.0), 1.33 (3H, t, J7.0). M/z (ES⁺) 342, 344 (M+H⁺).

### Description 12 6-(4-Chlorophenyl)-5-thioxo-5,6-dihydro[1,3]thiazolo[5,4]-d ]pyrimidin-7(4H)-one

Description 11 (358 mg, 1.05 mmol) was suspended in methanol (15 ml) at room temperature. Methanolic 2 M potassium hydroxide solution (2 ml, 2 mmol) was added and the reaction mixture was stirred for 3 h. The reaction was then cooled to 0° C. and acidified by adding 2 N aqueous hydrochloric acid (ca. 5 ml, 10 mmol). After stirring for 10 min, the solid was collected by filtration and washed with water (3×5 ml), then dried under vacuum to give the title compound (202 mg, 65%). ¹H NMR (400 MHz, DMSO) δ 13.87 (1H, br. s), 8.91 (1H, s), 7.55 (2H, d, J9.0), 7.32 (2H, d, J9.0).

### Description 13 Methyl 4-amino-1,3-thiazole-5-carboxylate

A suspension of methyl 4-amino-2-methylthio-1,3-thiazole-5-carboxylate (6.74 g, 33 mmol) and Raney-Nickel (commercially available slurry in water, ca. 15 ml, added in 5 portions throughout the reaction) in ethanol (200 ml) was hydrogenated at 45 psi for 1 week. The catalyst was removed by filtration, washed with ethyl acetate and ethanol and the filtrate evaporated. The resulting solid was purified by flash column chromatography [eluant:ethyl acetate/isohexane (1:4)] to give the title compound as a bright yellow solid (1.23 g, 24%). ¹H NMR (400 MHz, CDCl₃) δ 8.54 (1H, s), 5.90 (2H, brs), 3.84 (3H, s). M/z (ES⁺) 159 (M+H⁺).

### Description 14 Methyl 4-(4-chlorophenylaminocarbonothioylamino)-1,3-thiazole-5-carboxylate

Description 13 (1.23 g, 7.8 mmol), 4-chlorophenyl isothiocyanate (1.33 g, 7.8 mmol) and a catalytic amount of 4-dimethylaminopyridine in acetonitrile was refluxed at 100° C. for 18 h. The reaction was cooled and the solid collected by filtration, washed with acetonitrile and methanol to give the title compound (0.79 g, 31%). ¹H NMR (400 MHz, DMSO) δ 11.97 (1H, s). 10.13 (1H, s), 9.41 (1H, s), 7.71 (2H, d, J8.8), 7.48 (2H, d, J8.8), 3.89 (3H, s).

### Description 15 6-(4-Chlorophenyl)-5-thioxo-5,6-dihydro [1,3]thiazolo [4,5-d]pyrimidin-7(4H)-one

Description 14 (788 mg, 2.4 mmol) was suspended in methanol (5 ml). Methanolic 1 M potassium hydroxide (10 ml, 9.6 mmol) was then added and the reaction stirred at room temperature for 2 h. The insoluble material was filtered, and the filtrate cooled to 0° C. and acidified to pH 5 with 1 N aqueous hydrochloric acid and the resulting solid filtered and washed with water. The solid was dry loaded onto silica in acetonitrile/methanol and purified by flash column chromatography (eluant: 2.5% methanol in dichloromethane) to give the title compound as a pink solid (200 mg, 28%). ¹H NMR (400 MHz, DMSO) δ 14.37 (1H, s), 9.56 (1H, s), 7.54 (2H, d, J8.7), 7.31 (2H, d, J8.6). M/z (ES⁺) 296, 298 (M+H⁺).

### Description 16 1-(4-Chlorophenyl)-9-methyl-2-thioxo-1,2,3,9-tetrahydro-6H-purin-6-one

Ethyl 5-amino-1-methyl-1H-imidazole-4-carboxylate (Zhurnal Obshchei Khimii 1987, 57 (3), 692) (0.50 g, 2.96 mmol) and 4-chlorophenyl isothiocyanate (0.50 g, 2.96 mmol) were stirred in pyridine (2.5 ml) at 45° C. for 17 h. The reaction was cooled and diluted by the addition of ice. When the ice had melted the reaction was filtered, the product rinsed with water and dried to give ethyl 5-(4-chlorophenylaminocarbonothioylamino)-1-methyl-1H-imidazole-4-carboxylate (0.75 g, 75%). The solid was slurried in 1% aqueous sodium hydroxide solution (7.5 ml) and heated at 80° C. for 90 min. The reaction was cooled, diluted with methanol to dissolve all solids and loaded onto a strong cation exchange (SCX) cartridge. The cartridge was washed with methanol and then the product eluted with 2 M methanolic ammonia. The product was azeotroped with ethanol, triturated with acetonitrile and dried to give the title compound as an off white solid (0.63 g, 97%). ¹H NMR (360 MHz, DMSO) δ 7.58 (1H, s), 7.37 (2H, m), 7.06 (1H, brs), 6.96 (2H, m), 3.54 (3H, s). M/z (ES⁺) 293, 295 (M+H⁺).

### Description 17 Methyl-5-nitro-4-imidazolecarboxylate

Ammonium nitrate (3.2 g, 40.2 mmol) was added slowly to a solution of 4-imidazolecarboxylic acid (3.0 g, 26.8 mmol) in concentrated sulfuric acid (24 ml) at 100° C. The reaction was heated for 2 days then cooled. Methanol (15 ml) was added cautiously with vigorous stirring and then the reaction heated at 60° C. for 24 h. The reaction was cooled and poured onto ice, causing a fine white precipitate to form. The mixture was neutralized by the addition of 33% aqueous ammonia. The solid was filtered off and dried to give the title compound (1.24 g, 27%). A second crop of crystals was collected from the filtrate (0.82 g, 18%). ¹H NMR (400 MHz, DMSO) δ 14.2 (1H, brs), 7.94 (1H, s), 3.87 (3H, s).

### Description 18 Methyl-5-amino-4-imidazolecarboxylate

A solution of Description 17 (1.24 g, 7.25 mmol) in 1:1 ethanol:methanol (60 ml) was hydrogenated using 10% palladium on carbon catalyst under a balloon of hydrogen. After 4 h the reaction mixture was filtered, the filtrate condensed and azeotroped with ethanol. The product was triturated with ethyl acetate and dried to give the title compound as a white solid (0.98 g, 96%). ¹H NMR (400 MHz, DMSO) δ 12.0 (1H, brs), 7.32 (1H, s), 5.56 (2H, s), 3.70 (3H, s). M/z (ES⁺) 142 (M+H⁺).

### Description 19 1-(4-Chlorophenyl)-2-thioxo-1,2,3,7-tetrahydro-6H-purin-6-one

Description 18 (0.98 g, 6.95 mmol) and 4-chlorophenyl isothiocyanate (1.29 g, 7.65 mmol) were stirred in pyridine (5 ml) at 100° C. After 15 h additional 4-chlorophenyl isothiocyanate (0.12 g, 0.70 mmol) was added and heating continued for a further 4 h. The reaction was cooled, poured onto ice and the resultant solid, methyl 5-({[(4-chlorophenyl)amino]carbonothioyl}amino)-1H-imidazole-4-carboxylate, was collected by filtration and dried (0.42 g, 20%). Without purification, the solid was slurried in 1% aqueous sodium hydroxide solution (10 ml) and heated at 80° C. for 2 h. The reaction was cooled and filtered to remove unreacted starting material. The filtrate was acidified to pH 5 using acetic acid, causing a fine white precipitate to form. The solid was collected, rinsed with water and dried to give the title compound as a fine white solid (0.28 g, 73%). ¹H NMR (360 MHz, DMSO) δ 13.72 (2H, brs), 8.18 (1H, s), 7.55 (2H, m), 7.30 (2H, m). 11M/z (ES⁺) 279, 281 (M+H⁺).

### Description 20 2-Thioxo-3-[4-trifluoromethylphenyl-]2,3-dihydropyrido[3,2-d]pyrimidin-4(1H)-one

Description 4 (0.20 g, 1.31 mmol) and 4-trifiuoromethylphenyl isothiocyanate (0.32 g, 1.57 mmol) were heated at 75° C. in acetonitrile (5 ml) with a catalytic amount of 4-dimethylaminopyridine. After 16 h additional 4-(trifluoromethyl)phenyl isothiocyanate (50 mg, 0.25 mmol) was added and the reaction heated at 85° C. for a further 2 h. The reaction was cooled and the product collected by filtration, washed with acetonitrile (5 ml) and dried to give the title compound as a white solid (0.27 g, 90%). ¹H NMR (360 MHz, DMSO) δ 13.14 (1H, s), 8.61 (1H, m), 7.90-7.76 (4H, m), 7.57 (2H, d, J8.2). M/z (ES⁺) 324 (M+H⁺).

### Description 21 3-Pyridin-3-yl-2-thioxo-2,3-dihydropyrido[3,2-d]pyrimidin-4(1H)-one

3-Aminopyridine-2-carboxylic acid (Bioorg. Med. Chem. 2001, 9, 2061) (1.84 g, 13.3 mmol) was treated with 3-pyridyl isothiocyanate according to the method of Description 7 to give the title compound directly, as an off white solid (1.33 g, 38%). ¹H NMR (360 MHz, DMSO) δ 13.20 (1H, brs), 8.61 (2H, m), 7.80 (3H, m), 7.55 (1H, d, J1.9), 7.56 (1H, m). M/z (ES⁺) 257 (M+H⁺).

### Description 22 2-Chloro-N-(5-methylisoxazol-3-yl)acetamide

A solution of 3-amino-5-methylisoxazole (867 mg, 8.85 mmol) and triethylamine (2.4 ml, 17.7 mmol) in dichloromethane (10 ml) was added dropwise over 5 min to a solution of chloroacetyl chloride (0.707 ml, 8.85 mmol) in dichloromethane (15 ml) at 0° C. The solution was allowed to warm to room temperature and stir for a further 2 h. The solution was then washed with 1:1 brine:water (2×20 ml) and the dichloromethane layer dried over MgSO₄, filtered and evaporated. The resulting residue was triturated with diethyl ether to give the title compound (275 mg, 18%). 1H (360 MHz, DMSO) δ 11.25 (1 H, s), 6.62 (1 H, s), 4.29 (2 H, s), 2.38 (3 H, s). M/z(ES⁺) 175, 177 (M+H⁺).

### Description 23 1-(4-chlorophenyl)-9-cyclopropyl-2-thioxo-1,2,3,9-tetrahydro-6H-purin-6-one hydrochloride

To a solution of ethyl 3-nitriloalaninate (Synthesis, 1996, 11, 1325; 27 g, 0.21 mol) in MeCN (500 mL) was added triethylorthoformate (35 mL, 31.2 g, 0.25 mol) and the resulting solution heated to 90° C. After 90 min the yellow-green solution was cooled to room temperature and a solution of cyclopropyl amine (17.3 mL, 14.2 g, 0.25 mol) in EtOH (100 mL) was added, causing the solution to go orange. The reaction was stirred at 45° C. for 90 minutes then at room temperature overnight. The reaction was condensed to a viscous oil then taken up in dichloromethane (˜200 mL) and washed with sodium hydroxide solution (2M, 50 mL) then water (50 mL). The aqueous layers were combined and extracted with dichloromethane (2×100 mL). All the organic layers were combined, dried over MgSO₄ and condensed in vacuo to give a brown solid residue. The residue was slurried in minimum EtOH, filtered, the solid rinsed with ether and dried to give ethyl 5-amino-1-cyclopropyl-1H-imidazole-4-carboxylate as a beige solid (6.45 g, 16%). The filtrate also contained product. Ethyl 5-amino-1-cyclopropyl-1H-imidazole-4-carboxylate (4.0 g, 20.5 mmol) and 4-chlorophenyl isothiocyanate (3.47 g, 20.5 mmol) were stirred in pyridine (17 ml) at 45° C. for 24 h. The suspension was cooled and diluted by the addition of ice. When the ice had melted the reaction was filtered, the product rinsed with water and dried to give ethyl 5-(4-chlorophenyl aminocarbonothioylamino)-1-cyclopropyl-1H-imidazole-4-carboxylate (5.68 g). The solid was slurried in 1% aqueous sodium hydroxide solution (25 ml) and heated at 80° C. for 2 h. The reaction was filtered to remove insoluble impurities and then acidified to pH˜5 using hydrochloric acid (5N), causing a thick white suspension to form. The mixture was aged for 30 minutes, diluted with water and filtered. The solid was rinsed with water then ether and dried to give the title compound as a beige solid (3.95 g, 61%). ¹H NMR (360 MHz, DMSO) δ 7.88 (1H, s), 7.52 (2H, J8.6), 7.22 (2H, J8.6), 3.47-3.45 (1H, m),1.08 (4H, d, J6.9). M/z (ES⁺) 319, 321 (M+H⁺).

### Description 24 1-(4-chlorophenyl)-9-phenyl-2-thioxo-1,2,3,9-tetrahydro-6H-purin-6-one hydrochloride

Prepared from ethyl 3-nitriloalaninate and aniline according to the procedure described in Description 23. ¹H NMR (360 MHz, DMSO) δ 8.08 (1H, s), 7.61-7.53 (7H, m), 7.24 (2H, d, J8.6). M/z (ES⁺) 400, 402 (M+H⁺).

### Description 25 1-(4-chlorophenyl)-9-ethyl-2-thioxo-1,2,3,9-tetrahydro-6H-purin-6-one hydrochloride

Prepared from ethyl 3-nitriloalaninate and ethylamine according to the procedure described in Description 23. ¹H NMR (400 MHz, DMSO) δ 13.90 (1H, s), 7.95 (1H, s), 7.54-7.50 (2H, m), 7.26-7.22 (2H, m), 4.23 (2H, q, J7.2), 1.35 (3H, J7.2). M/z (ES⁺) 307, 309 (M+H⁺).

### Description 26 2-chloro-1-(4-chlorophenyl-9-ethyl-1,9-dihydro-6H-purin-6-one

A solution of Description 25 (860 mg, 2.5 mmol) in phosphorous oxychloride (4.5 ml, 20 eq) was stirred at 110° C. for 36 h. The reaction mixture was cooled, evaporated in vacuo, and azeotroped twice with toluene. The resulting sticky brown oil was then neutralized with sat. NaHCO₃ (aq) and the resulting solid collected by filtration. The crude solid was dissolved in dichloromethane and purified by flash column chromatography on silica [eluant:ethyl acetate/dichloromethane (1:1)] to give the title compound as pale yellow solid (426 mg, 55%). ¹H NMR (500 MHz; CDCl₃) δ 7.79 (1H, s), 7.52 (2H, d, J8.6), 7.21 (2H, d, J 8.6), 4.23 (2H, q, J7.3), 1.56 (3H, t, J7.3). M/z (ES⁺) 309, 311 (M+H⁺).

### Description 27 2-[2-fluoro-4-(trifluoromethyl)phenyl]ethanamine

To a suspension of sodium borohydride (528 mg, 13.9 mmol) in tetrahydrofuran (10 ml) was added trifluoroacetic acid (1.6 g, 13.9 mmol) dropwise at room temperature over 10 mins to give a solution of sodium trifluoroacetoxyborohydride [NaBH₃(OCOCF3)]. To this was added a solution of 2-fluoro-4-(trifluoromethyl)phenylacetonitrile (2.83 g, 13.9 mmol) in tetrahydrofuran (5 ml) and the resulting solution stirred at RT for 20 hrs. The reaction was quenched by the addition of water (1 ml) and then evaporated in vacuo and the resulting oil was dissolved in dichloromethane and loaded onto a strong cation exchange (SCX) cartridge. The cartridge was washed with dichloromethance and methanol then the product eluted with 2M ammonia in methanol. This gave the title compound as a brown oil (900 mg, 31%). ¹H NMR (360 MHz; DMSO) δ 7.65-7.55 (3H, m), 5.89-5.45 (2H, br s), 2.98-2.88 (4H, m). M/z (ES⁺) 208 (M+H⁺).

### Description 28 4-(9-methyl-6-oxo-2-thioxo-2,3,6,9-tetrahydro-1H-purin-1-yl)benzonitrile hydrochloride

Prepared from ethyl 3-nitriloalaninate, methylamine and 4-cyanophenyl isothiocyanate, according to the procedure described in Description 23. ¹H NMR (500 MHz, DMSO) δ 14.00 (1H, s), 7.96 (2H, d, J8.4), 7.87 (1H, s), 7.45 (2H, d, J 8.4), 3.77 (3H, s). M/z (ES⁺) 284 (M+H⁺).

### Description 29 9-methyl-1-(4-methylphenyl)-2-thioxo-1,2,3,9-tetrahydro-6H-purin-6-one hydrochloride

Prepared from ethyl 3-nitriloalaninate, methylamine and 4-tolyl isothiocyanate, according to the procedure described in Description 23. ¹H NMR (360 MHz, DMSO) δ 7.83 (1H, s), 7.25 (2H, d, J8.1), 7.02 (2H, d, J8.1), 3.75 (3H, s) 2.36 (3H, s). M/z (ES⁺) 273 (M+H⁺).

### Description 30 General Procedure for Preparation of Phenethyl Bromides

Borane dimethylsulifide complex (2M in THF, 6.9 ml, 13.8 mmol) was added slowly to a solution of the appropriate phenylacetic acid (10.6 mmol) in THF (20 ml) at room temperature. The reaction was stirred overnight before being quenched by the slow addition of sodium hydroxide (2N, 20 ml). The mixture was stirred at room temperature for 1 h then extracted with ethyl acetate (2×50 ml). The organic layers were dried over MgSO₄ and condensed to give the appropriate alcohol as an oil (quantitative). Phosphorous tribromide (0.50 ml, 1.4 g, 5.3 mmol) was added to the neat alcohol whilst stirring in a room temperature water bath. The reaction was then heated to 100° C. for 2 h until evolution of HBr ceased. The reaction was cooled and added dropwise to an ice/water mixture. The product was extracted twice with hexane and the combined organic layers washed with saturated sodium carbonate solution. The organic layer was dried over MgSO₄ and condensed to give the desired bromide which was used without further purification.

The following alkyl bromides were made according to the general procedure described in Description 30: 1-(2-bromoethyl)-4-trifluoromethylbenzene; 1-(2-bromoethyl)-2,4-dichlorobenzene; 1-(2-bromoethyl)-2-fluoro-4-trifluoromethylbenzene; 4-(2-bromoethyl)-2-fluoro-1-trifluoromethylbenzene; 1-(2-bromoethyl)-2-chloro-4-trifluoromethylbenzene; 1-(2-bromoethyl)-3-chlorobenzene.

3-bromomethyl-6-chloro-1-benzothiophene was prepared according to Magn. Reson. Chem. 1985, 23, 10, 814.

### Description 31 [2-Chloro-4-trifluoromethylphenyl]acetic acid

Trifluoroacetic acid (5 ml) was added to a solution of tertbutyl [2-chloro-4-trifluoromethylphenyl ] acetate (US-A-6620838; J Am. Chem. Soc. 2002, 124, 12557; 3.64 g, 12.4 mmol) in CH₂Cl₂ (30 ml) and the reaction was stirred at room temperature for 4 h. The reaction was condensed to approximately half the volume and additional TFA (2 ml) was added and the reaction stirred for a further 3 h. The reaction was evaporated to dryness and taken on, without characterization, to the procedure described in Description 30.

### Description 32 2-Allyl-5-trifluoromethylophenyl

Allyl-(3-trifluoromethylphenyl)-ether (J. Am. Chem. Soc. 1951, 73, 2375, 10 g, 0.05 mol) was irradiated for 90 min at 240° C. in a Smith Optimiser microwave to give a 1:1 mixture of two isomers. Column chromatography, eluting with 4 to 10% ethyl acetate in hexane gave the desired isomer, 2-allyl-5-trifluoromethylphenol, as the less polar component (3.8 g, 38%). ¹H NMR (360 MHz, CDCl₃) δ 7.22 (1H, d, J7.8), 7.14 (1H, t, J7.8), 7.05 (1 H, 8), 6.04-5.96 m), 5.26 (1H, s), 5.21-5.15 (2H, m), 3.45 (2H, d, J6.3).

### Description 33 2-(2-Hydroxyethyl)-5-trifluoromethylphenol

To a solution of Description 32 (2.0 g, 0.01 mol) in CH₂C₂ (50 ml) was added MeOH (30 ml) and the resulting solution was cooled to −78° C. and bubbled with nitrogen for 10 min. The reaction was perfused with oxygen for 10 min at −78° C. then with ozone until the solution turned blue. Sodium borohydride (0.75 g) was added and the reaction stirred at −78° C. After 80 min additional sodium borohydride (0.75 g) was added and the reaction allowed to warm to room temperature overnight. The reaction was quenched by the addition of acetone followed by water and condensed. The reaction was partitioned between HCl (1N, 50 ml) and CH₂Cl₂ (3×50 ml). The combined organic layers were dried over MgSO₄ and condensed to give the title compound (1.53 g, 75%). ¹H NMR (360 MHz, CDCI₃) δ 7.13 (4H, m), 4.04 (2H, t, J5.2), 2.95 (2H, t, J5.2).

### Description 34 2-(2-Iodoethyl)-5-trifluoromethylphenol

A solution of Description 33 (0.75 g, 3.64 mmol) in CH₂Cl₂ (10 ml) was added to a suspension of triphenylphosphine (1.05 g, 4.00 mmol), imidazole (0.27 g, 4.0 mmol) and iodine (1.02 g, 4.00 mmol) in CH₂C₁₂ (10 ml) at 0° C. The reaction was allowed to warm to room temperature over 2 h. The reaction was diluted with CH₂Cl₂ and shaken with saturated sodium thiosulfate solution. The aqueous later was extracted with CH₂Cl₂ (3 ×). The combined organic layers were dried over MgSO₄ and condensed to give a crude product which was purified by column chromatography, eluting with 10% ethyl acetate in hexane, to give the title compound (0.85 g, 74%). ¹H NMR (360 MHz, CDCl₁₃) δ 7.23 (1H, s), 7.16 (1H, d, J 7.8), 7.00 (1H, s), 5.28 (1H, s), 3.43-3.39 (2H, m), 3.25 (2H, t, J7.5).

### Description 35 Ethyl 2-trifluoromethyl-1.3-thiazole-4-carboxylate

A solution of 2,2,2-trifluoroacetamide (7.12 g, 63 mmol) and Lawesson's Reagent (15.3 g, 37.8 mmol) in THF (anhydrous, 60 ml) was stirred at reflux for 18 h. The reaction mixture was cooled, then ethyl bromopyruvate (8 ml, 63 mmol) added, and refluxed for 18 h. The reaction was cooled, evaporated in vacuo, and the resulting crude material extracted into ethyl acetate and washed with water. The organic fraction was dried over MgSO₄, and condensed to give a yellow/orange oil. The residue was purified by flash column chromatography on silica eluting with 15% ethyl acetate in hexane to provide the title compound as a clear oil (3 g, 21%). ¹H NMR (400MHz, CDCl₁) δ 8.39 (1H, s), 4.47 (2H, q, J7.1), 1.42 (3H, t, J7.2).

### Description 36 4-Bromomethyl-2-trifluoromethyl-1,3-thiazole

Diiosbutyl aluminium hydride (1M in dichloromethane, 25.2 ml, 25.2 mmol) was added dropwise to a solution of Description 35 (2.83 g, 12.6 mmol) in THF (anhydrous, 40 ml) at −78° C. This was allowed to stir at −78° C. for 1 h then a further equivalent of diisobutyl aluminium hydride (1M in dichloromethane, 12 ml, 12 mmol) was added and the solution stirred at −78° C. for another hour. Methanol (20 ml) was added to the solution and allowed to warm to room temperature. The reaction was evaporated in vacuo, extracted into diethyl ether, washed with aqueous sodium potassium tartrate (200 ml), then aqueous ammonium chloride (200 ml). The diethyl ether layer was dried over MgSO₄, evaporated in vacuo to give the crude alcohol product as a light yellow oil (2.9 g). Bromine (˜800 μl) was added dropwise to a light yellow solution of the crude alcohol (2 g, 11 mmol) and triphenyl phosphine (3.15 g, 12 mmol) in N,N-dimethylformamide (anhydrous, 20 ml) at 0° C. until the colour persisted. This was then stirred at RT for 1 h. The solution was extracted into ethyl acetate, washed with water, dried over MgSO₄, evaporated in vacuo to give a yellow solid. This was purified by flash column chromatography on silica eluting with 10% ethyl acetate in hexane to give the title compound as a yellow oil (1.64 g, 61%). 1H NMR (400 MHz, DMSO) δ 8.24 (1H, s), 4.84 (2H, s).

### Description 37 1-[2-Trifluoromethyl-1,3-thiazol-4-yl]methanamine

Potassium phthalimide (495 mg, 2.7 mmol) was added to a solution of Description 36 (470 mg, 1.9 mmol) in N,N-dimethylformamide (anhydrous, 7 ml). The resulting suspension was stirred at room temperature overnight. The resulting solid was filtered, and the filtrate extracted into ethyl acetate, washed with water, dried over MgSO₄, evaporated in vacuo to give a white solid (520 mg, 1.67 mmol). This was dissolved in tetrahydrofuran/ethanol (10 ml/15 ml), hydrazine hydrate (600 μl, 10.4 mmol) added, and the reaction stirred at room temperature overnight. To the resulting suspension was added hydrochloric acid (10N, 10 μl) and the mixture filtered. The filtrate was basified to pH 10 with solid NaOH, and extracted into DCM. The DCM layer was dried over MgSO₄, evaporated in vacuo to give the title compound as a yellow oil (170 mg, 52%). ¹H NMR (400 MHz, DMSO) δ 7.88 (1H, s), 3.88 (2H, s). M/z (ES⁺) 183 (M+H⁺).

### Description 38 [4-Trifluoromethyl-1,3-thiazol-2-yl]methanol

A solution of 3-bromo-1,1,1-trifluoroacetone (3.2 ml, 30.2 mmol) and 2-amino-2-thioxoethyl private (5.3 g, 30.2 mmol) in ethanol (15 ml) was stirred at reflux for 18 h. To the cooled solution were added methanol (10 ml) and DBU (4.6 ml, 30.2 mmol) and the solution was stirred at room temperature for 2 days. The reaction mixture was evaporated in vacuo, extracted with dichloromethane, washed with water, dried over Na₂SO₄, and evaporated in vacuo to give crude product as a black oil. This was purified by flash column chromatography on silica eluting with 30% ethyl acetate in hexane to give the title compound as an off-white solid (3.2 g, 58%). ¹H NMR (400 MHz, DMSO) δ 8.42 (1H, s), 6.30 (1H, t, J5.8), 4.79 (2H, d, J5.7).

### Description 39 2-Bromomethyl-4-trifluoromethyl-1,3-thiazole

Bromine (˜500 μl) was added dropwise to a solution of Description 38 (1.2 g, 6.6 mmol) and triphenyl phosphine (1.9 g, 7.2 mmol) in N,N-dimethylformamide (anhydrous, 10 ml) at 0° C. until the colour persisted. This was then stirred at room temperature for 30 min. The solution was extracted into ethyl acetate, washed with water (×4), dried over Na₂SO₄ and evaporated in vacuo to give a yellow oil/solid. This was purified by flash column chromatography on silica eluting with 30% ethyl acetate in hexane to give the title compound as a yellow oil (1.1 g, 68%). ¹H NMR δ (500 MHz, DMSO) δ 8.56 (1H, s), 5.08 (2H, s).

### Description 40 3-Bromo-4-chloroaniline

3-Bromo-4-chloronitrobenzene (2.01 g, 8.50 mmol) was added to an efficiently stirred mixture of iron powder (2.37 g) and 1N aqueous hydrochloric acid (18 ml) at 40° C. The mixture was then warmed to 85° C. for 2 h. After cooling to RT the mixture was basified by addition of 10% aqueous potassium carbonate solution. Ethyl acetate (80 ml) was then added and the mixture filtered through a glass-fibre pad. The layers were separated and the aqueous phase extracted with more ethyl acetate (70 ml). The combined organic layers were dried over sodium sulphate and evaporated to give the title compound. ¹H NMR (500 MHz, CDCl₃) δ 7.17 (1H, d, J8.5), 6.94, (1H, d, J2.65), 6.54 (1H, dd, J8.5, 2.65), 3.70 (2H, br. s).

### Description 41 2-Chloro-5-nitrophenol

A mixture of 2-chloro-5-nitroanisole (101.86 g, 543 mmol), and 48% hydrobromic acid (500 ml) in acetic acid (500 ml) was heated at reflux for 3 days. Further 48% hydrobromic acid (200 ml) was added after 48 hours. The mixture was cooled and poured onto ice/water (3 litres). The resultant solid was removed by filtration. The solid was taken up into 1N NaOH (1 litre), and washed with EtOAc (3×500 ml). The aqueous layer was acidified by the addition of conc. HCl with cooling. The mixture was extracted with EtOAc (3×500 ml), the combined EtOAc layers washed with water (500 ml), sat. NaCl (500 ml), dried over Na₂SO₄, filtered and evaporated to give a beige solid (51 g, 54%). ¹H NMR (400 MHz, DMSO-d₆) 11.29 (1H, s), 7.77 (1H, d, J2.5), 7.67 (1H, dd, J8.7 and 2.5), 7.63 (1H, d, J8.7).

### Description 42 1-Chloro-2-ethoxy-4-nitrobenzene

To a solution of Description 41 (5.0 g, 28.8 mmol) in anhydrous N,N-dimethylformamide (50 ml) was added portionwise sodium hydride (60% dispersion in oil, 1.73 g, 43.2 mmol). The mixture was stirred at room temperature for 10 min, then iodoethane (3.46 ml, 43.2 mmol) added, and stirring continued for 3 days. The mixture was poured into water (200 ml) and extracted with EtOAc (200 ml). The organic layer was washed with water (250 ml), sat. NaCl (100 ml), dried over Na₂SO₄, filtered, and evaporated to give a dark oil (5.8 g, quant). ¹H NMR (400 MHz, CDCl₃) 7.75 (2H, m), 7.49 (1H, dd, J8.2 and 0.5), 4.21 (2H, q, J7.0), 1.52 (3H, t, J7.0).

### Description 43 (4-Chloro-3-ethoxyphenyl)amine

To a suspension of Description 42 (6.49 g, 32.2 mmol) in a mixture of glacial acetic acid (40 ml) and water (100 ml), mixed with an overhead stirrer was added iron powder (8.99 g, 161 mmol) and the mixture heated at reflux for 1 hour. The mixture was cooled and filtered through hyflo supercel®. The solid was washed with EtOAc (200 ml) and water (200 ml) and the organic layer separated, washed with more water (300 ml), sat. K₂CO₃ (100 ml), sat. NaCl (100 ml), dried over Na₂SO₄, filtered and evaporated. The residue was purified by column chromatography on silica (eluent: 100% dichloromethane) to give a dark oil (4.01 g, 72%). ¹H NMR (400 MHz, CDCl₃) 7.07 (1H, d, J8.4), 6.25 (1H, d, J2.6), 6.19 (1H, dd, J8.4 and 2.6), 4.02 (2H, q, J7.0), 3.60 (2H, br s), 1.43 (3H, t, J7.0).

### Description 44 (4-Chloro-3-isopropylphenyl)amine

Prepared from Description 41 and 2-iodopropane according to the procedures of Descriptions 42 and 43. ¹H NMR (400 MHz, CDCl₃) 7.07 (1H, d, J8.4), 6.28 (1H, d, J2.5), 6.20 (1H, dd, J8.4 and 2.5), 4.45 (1H, septet, J6.0), 3.61 (2H, br s), 1.34 (6H, d, J6.0).

(4-Chloro-3-methoxyphenyl)amine can be prepared according to Environ. Toxicol. Chem. 2001, 20, 7, 1381.

### Description 45 Ethyl 5-isothiocyanato-1-methyl-1H-imidazole-4-carboxylate

A solution of ethyl 5-amino-1-methyl-1H-imidazole-4-carboxylate (Zhurnal Obshchei Khimii 1987, 57 (3), 692) (100 mg, 0.59 mmol) and 1,1′-thiocarbonyldi-2(1)H-pyridone (137 mg, 0.59 mmol) in CH₂Cl₂ (3 ml) was stirred at room temperature for 22 h. The reaction was condensed and purified by flash column chromatography, eluting with ethyl acetate, to give the title compound (78 mg, 63%). 1H NMR (360 MHz, CDCl₃) δ 7.31 (1H, s), 4.42 (2H, q, J7.1), 3.60 (3H s), 1.41 (3H, t, J7.1).

### Description 46 Ethyl 5-isothiocyanato-1-ethyl-1H-imidazole-4-carboxulate

Prepared from ethyl 5-amino-1-ethyl-1H-imidazole-4-carboxylate (made from ethylamine and ethyl 3-nitriloalaninate according to procedure described in Description 23) and 1,1′-thiocarbonyldi-2(1H-pyridone according to procedure described in Description 45. ¹H NMR (360 MHz, CDCl₃) δ ¹H NMR (400 MHz, CDCl₃) 7.38 (1H, s), 4.05 (2H, q, J7.2), 3.97 (2H, q, J7.4), 1.47 (3H, t, J7.4), 1.41 (3H, t, J7.2).

### Description 47 1-(4-Chloro-3-fluorophenyl)-9-methyl-2-thioxo-1,2,3,9-tetrahydro-6H-purin-6-one hydrochloride

Description 45 (78 mg, 0.37 mmol) and 3-fluoro-4-chloroaniline (54 mg, 0.37 mmol) in MeCN (2 ml) in the presence of a catalytic quantity of DMAP were stirred at 45° C. for 16 h. The reaction was cooled and the resultant solid collected by filtration and rinsed with ether. Without further purification this solid was added to sodium hydroxide solution (1% aqueous w/w, 2 ml) and heated at 80° C. for 30 min. The reaction was cooled, filtered through Celite® to remove insoluble impurities and the filtrate was acidified to pH ˜6 by the dropwise addition of hydrochloric acid (5N) to precipitate the product. The solid was collected by filtration, rinsed with water then ether and dried to give the title compound (70 mg). ¹H NMR (400 MHz, DMSO) δ 7.86 (1H, s), 7.70 (1H, t, J8.4), 7.43 (1H, dd, J2.2, 10.0), 7.14-7.12 (1H, m), 3.77 (3H, s); Mz (ES⁺) 313, 311 (M+H⁺).

### Description 48 1-(4-Chloro-3-ethoxyphenyl)-9-ethyl-2-thioxo-1,2,3,9-tetrahydro-6H-purin-6-one

Prepared from Description 43 and Description 46 according to the procedure of Description 47. ¹H NMR (400 MHz, DMSO-d₆) 13.87 (1H, br s), 7.94 (1H, s), 7.49 (1H, d, J7.7), 7.06 (1H, s), 6.80 (1H, d, J7.7), 4.23 (2H, br m), 4.07 (2 H, br m), 1.34 (6H, m).

### Description 49 1-(4-Chloro-3-isopropoxyhenyl)-9-ethyl-2-thioxo-1,2,3,9-tetrahydro-6H-purin-6-one

Prepared from Description 44 and Description 46 according to the procedure of Description 47. ¹H NMR (400 MHz, DMSO-d₆) 13.80 (1H, br s), 7.95 (1H, s), 7.47 (1H, d, J6.9), 7.08 (1H, s), 6.78 (1H, d, J6.9), 4.57 (1H, br m), 4.23 (2H, br m), 1.31 (9H, m).

### Description 50 3-(6-Chloropyridin-3-yl)-2-thioxo-2,3-dihydrothieno[3,2-d]pyrimidin-4(1H)-one

A solution of methyl 3-isothiocyanatothiophene-2-carboxylate (200 mg, 1.0 mmol), 6-chloropyridin-3-amine (129 mg, 1.0 mmol) and a catalytic amount of DMAP in MeCN (5 ml) was stirred at 70° C. overnight. The reaction was cooled, condensed and partitioned between water and CH₂Cl₂ (2×). The organic layers were combined, dried over MgSO₄ and condensed to give a brown oil which was used without further purification. M/z (ES⁺) 298, 296 (M+H⁺).

The following descriptions, 51 to 74 as shown below, were made by procedures analogous to those described above.

| | | | Made |
|---|---|---|---|
| | | M/z ES⁺ | according |
| Description | Name | [M + H⁺] | to procedure of |
| 51 | 1-(4-chlorophenyl)-9- | 323, 321 | Description 23 |
| | propyl-2-thioxo-1,2,3,9- | | |
| | tetrahydro-6H-purin-6-one | | |
| | hydrochloride | | |
| 52 | 1-(4-chlorophenyl)-9-(2- | 325, 323 | Description 23 |
| | hydroxyethyl)-2-thioxo- | | |
| | 1,2,3,9-tetrahydro-6H- | | |
| | purin-6-one hydrochloride | | |
| 53 | 4-(9-ethyl-6-oxo-2-thioxo- | 298 | Description 23 |
| | 2,3,6,9-tetrahydro-1H- | | |
| | purin-1-yl)benzonitrile | | |
| | hydrochloride | | |
| 54 | 1-(4-fluorophenyl)-9- | 277 | Description 23 |
| | methyl-2-thioxo-1,2,3,9- | | |
| | tetrahydro-6H-purin-6-one | | |
| | hydrochloride | | |
| 55 | 9-ethyl-1-(4-fluorophenyl)- | 291 | Description 23 |
| | 2-thioxo-1,2,3,9-tetrahydro- | | |
| | 6H-purin-6-one | | |
| | hydrochloride | | |
| 56 | 1-(3-chloro-4-fluorophenyl)- | 313, 311 | Description 23 |
| | 9-methyl-2-thioxo-1,2,3,9- | | |
| | tetrahydro-6H-purin-6-one | | |
| | hydrochloride | | |
| 57 | 1-(3,4-difluorophenyl)-9- | 295 | Description 23 |
| | methyl-2-thioxo-1,2,3,9- | | |
| | tetrahydro-6H-purin-6-one | | |
| | hydrochloride | | |
| 58 | 1-(2,4-dichlorophenyl)-9- | 329, 327 | Description 23 |
| | methyl-2-thioxo-1,2,3,9- | | |
| | tetrahydro-6H-purin-6-one | | |
| | hydrochloride | | |
| 59 | 9-cyclopropyl-1-(4- | 303 | Description 23 |
| | fluorophenyl)-2-thioxo- | | |
| | 1,2,3,9-tetrahydro-6H- | | |
| | purin-6-one hydrochloride | | |
| 60 | 1-(4-bromophenyl)-9-ethyl- | 350, 352 | Description 23 |
| | 2-thioxo-1,2,3,9-tetrahydro- | | |
| | 6H-purin-6-one | | |
| | hydrochloride | | |
| 61 | 9-ethyl-2-thioxo-1-[4- | 357 | Description 23 |
| | trifluoromethoxyphenyl]- | | |
| | 1,2,3,9-tetrahydro-6H-purin- | | |
| | 6-one hydrochloride | | |
| 62 | 9-ethyl-1-(4-methylphenyl)- | 287 | Description 23 |
| | 2-thioxo-1,2,3,9-tetrahydro- | | |
| | 6H-purin-6-one | | |
| | hydrochloride | | |
| 63 | 1-[4-dimethylaminophenyl]- | 316 | Description 23 |
| | 9-ethyl-2-thioxo-1,2,3,9- | | |
| | tetrahydro-6H-purin-6-one | | |
| | hydrochloride | | |
| 64 | 9-methyl-2-thioxo-1-[4- | 327 | Description 23 |
| | trifluoromethylphenyl]- | | |
| | 1,2,3,9-tetrahydro-6H-purin- | | |
| | 6-one hydrochloride | | |
| 65 | 1-(3-chlorophenyl)-9-methyl- | 295, 293 | Description 23 |
| | 2-thioxo-1,2,3,9-tetrahydro- | | |
| | 6H-purin-6-one | | |
| | hydrochloride | | |
| 66 | 1-(4-chlorophenyl)-9- | 332, 324 | Description 23 |
| | cyclopropylmethyl-2-thioxo- | | |
| | 1,2,3,9-tetrahydro-6H-purin- | | |
| | 6-one hydrochloride | | |
| 67 | 1-(4-chlorophenyl)-9- | 321, 323 | Description 23 |
| | isopropyl-2-thioxo-1,2,3,9- | | |
| | tetrahydro-6H-purin-6-one | | |
| | hydrochloride | | |
| 68 | 3-phenyl-2-thioxo-2,3- | 256 | Description 5 |
| | dihydropyrido[3,2- | | |
| | d]pyrimidin-4(1H)-one | | |
| 69 | 3-(4-fluorophenyl)-2-thioxo- | 273 | Description 5 |
| | 2,3-dihydropyrido[3,2- | | |
| | d]pyrimidin-4(1H)-one | | |
| 70 | 4-(4-oxo-2-thioxo-1,4- | 281 | Description 5 |
| | dihydropyrido[3,2- | | |
| | d]pyrimidin-3(2H)- | | |
| | yl)benzonitrile | | |
| 71 | 2-thioxo-3-[4- | 329 | Description 8 |
| | trifluoromethylphenyl]-2,3- | | |
| | dihydrothieno[3,2- | | |
| | d]pyrimidin-4(1H)-one | | |
| 72 | 3-(4-chlorophenyl)-7- | 308, 310 | Description 8 |
| | methyl-2-thioxo-2,3- | | |
| | dihydrothieno[3,2- | | |
| | d]pyrimidin-4(1H)-one | | |
| 73 | 1-(4-chloro-3- | 325, 323 | Description 47 |
| | methoxyphenyl)-9-methyl- | | |
| | 2-thioxo-1,2,3,9-tetrahydro- | | |
| | 6H-purin-6-one | | |
| 74 | 1-(3-bromo-4-chlorophenyl)- | 371, 373, | Description 47 |
| | 9-methyl-2-thioxo-1,2,3,9- | 375 | |
| | tetrahydro-6H-purin-6-one | | |

### Description 75 2-Chloro-1-(4-chlorophenyl)-9-methyl-1,9-dihydro-6H-purin-6-one

Prepared from Description 16 according to the procedure described in Description 26.

¹H NMR (360 MHz, DMSO) δ 8.14 (1H, s), 7.64 (2H, d, J8.6), 7.52 (2H, d, J8.6), 3.76 (3H, s). M/z (ES⁺) 295, 297 (M+H⁺).

### Description 76 2-Chloro-3-(4-chlorophenyl)-7-methylthieno[3,2-d]pyrimidin-4(3H)-one

A mixture of Description 72 (1.1 g, 3.56 mmol) and phosphorous oxychloride (16.6 ml, 178 mmol) were heated at 105° C. for 4 h. The mixture was allowed to cool, and the excess phosphorous oxychloride removed by evaporation. The residue was taken up in dichloromethane (100 ml) and ice (100 g) added, and the resulting mixture stirred for 30 min. The organic layer was separated, dried over Na₂SO₄, filtered and evaporated to give a dark solid (1.0 g, 90%). ¹H NMR (400 MHz, CDCl₃) 7.51 (3H, m), 7.22 (2H, m), 2.41 (3H, d, J1.1); m/z (ES⁺) 311 (M+H⁺).

### EXAMPLE 1

### 3-(4-Chlorophenyl)-2-[3-fluorobenzylthio]pyrido[3,4-d]pyrimidin-4(3H)-one

A suspension of Description 3 (0.50 g, 1.73 mmol), potassium carbonate (1.20 g, 8.70 mmol) and 3-fluorobenzyl bromide (0.34 g, 1.82 mmol) in acetonitrile (12 ml) was stirred at room temperature for 1 h. Additional 3-fluorobenzyl bromide (34 mg, 0.18 mmol) was added and the reaction stirred for a further hour. The reaction was diluted with water (50 ml), extracted with dichloromethane (2×50 ml) and the combined organic fractions dried over MgSO₄ and condensed. The crude product was purified by flash column chromatography, eluting with 2% methanol in dichloromethane, to give the title compound as a white solid (100 mg, 15%). ¹H NMR (400 MHz, CDCl₃) δ 9.10 (1H, s), 8.65 (1H, d, J5.2), 7.99 (1H, dd, J5.3, 0.8), 7.52 (2H, m), 7.23 (3H, m), 7.16 (1H, d, J7.8), 7.10 (1H, m), 6.95 (1H, m), 4.42 (2H, s). M/z (ES⁺) 398, 400 (M+H⁺).

### EXAMPLE 2

### 3-(4-Chlorophenyl)-2-{2-(4-chlorolhenyl)-2-oxoethylthio}pyrido [3,2]pyrimidin-4(3H)-one

A suspension of Description 5 (75 mg, 0.26 mmol), potassium carbonate (75 mg, 0.54 mmol) and 2-bromo-4′-chloroacetophenone (67 mg, 0.29 mmol) in acetonitrile (4 ml) was stirred at 75° C. for 5 h. The reaction was cooled and diluted with water (ca. 7 ml) to dissolve the salts. The solid was collected by filtration, rinsed with water (3 ml) then diethyl ether (5 ml) and dried to give the title compound as an off white solid (48 mg, 44%). ¹H NMR (500 MHz, DMSO) δ 8.69 (1H, m), 8.10 (2H, m), 7.75-7.66 (5H, m), 7.57 (2H, m), 7.54 (1H, m), 4.77 (2H, s). M/z (ES⁺) 442, 444 (M+H⁺).

### EXAMPLES 3-48

Examples 3-46 were prepared using the appropriate purinone or pyrimidinone core (Descriptions 5, 7, 8, 12, 15, 16, 19, 20, 21, 23-25, 28 and 29) and the appropriate alkyl iodide, bromide or chloride in a procedure analogous to Example 2. Alkyl iodides, bromides and chlorides are commercially available or described in Description 22 or prepared by known methods as follows: 1-(2-bromoethyl)-4-trifluoromethyl benzene, Can. J. Chem. 1996, 74, 453; 3-bromomethylbenzo[b]thiophene, J. Med. Chem. 2002, 45, 4559; 4-(2-bromoethyl)chlorobenzene, J. Am. Chem. Soc. 1977, 99, 3059. Where the product did not precipitate analytically pure from the reaction it was purified by recrystallisation, flash column chromatography, preparative thin layer chromatography or mass directed HPLC as appropriate.

| | | M/z | |
|---|---|---|---|
| EX | NAME | ES+ [M + H⁺] | ¹H NMR |
| 3 | 3-(4-chlorophenyl)-2-[3 | 398, 400 | (400MHz, DMSO) δ |
| | fluorobenzylthiol]pyrido | | 8.75(1H, dd, J4.3, 1.5), |
| | [3,2-d]pyrimidin-4(3H)- | | 8.12(1H, dd, J8.2, 1.5), |
| | one | | 7.85(1H, dd, J8.2, 4.3), |
| | | | 7.64(2H, d, J8), 7.55(2H, d, |
| | | | J8), 7.35-7.25(3H, m), |
| | | | 7.10-7.00(1H, m), |
| | | | 4.45(2H, s). |
| 4 | 3-(4-chlorophenyl)-2-{2- | 428, 430 | (400MHz, DMSO) δ |
| | (4-chlorophenyl)ethylthio} | | 2.94-2.96(2H, m), |
| | pyrido[3,2-d] | | 3.34-3.37(2H, m), 7.31-7.37(4H, |
| | pyrimidin-4(3H)-one | | m), 7.53(2H, d, J8.6), |
| | | | 7.65(2H, d, J8.6), 7.85(1H, |
| | | | dd, J4.3, 8.2), 8.08(1H, |
| | | | dd, J1.6, 8.2), 8.75(1H, |
| | | | dd, J1.6, 4.3). |
| 5 | 2-{5-chloro-1-benzothien- | 470, 472 | (400MHz, DMSO) d |
| | 3-ylmethylthio}-3-(4- | | 4.71(2H, s), 7.42(1H, dd, |
| | chlorophenyl)pyrido[3,2- | | J1.6, 8.6), 7.52(2H, d, |
| | d]pyrimidin-4(3H)-one | | J11.5), 7.63(2H, d |
| | | | J11.5), 7.88(1H, dd, |
| | | | J4.3, 8.2), 8.01(2H, m), |
| | | | 8.08(1H, d, J2.0), 8.23(1H, |
| | | | dd, J1.6, 8.2), 8.76(1H, |
| | | | dd, J1.6, 4.3). |
| 6 | 2-[1-benzothien-3- | 436, 438 | (360MHz, CDCl₃) d |
| | ylmethylthio]-3-(4- | | 8.82(1H, dd, J4.2, 1.8), |
| | chlorophenyl)pyrido[3,2- | | 8.08(1H, dd, J8.4, 1.2), |
| | d]pyrimidin-4(3H)-one | | 7.85(1H, m), 7.79(1H, m), |
| | | | 7.69(1H, dd, J8.4, 4.1), |
| | | | 7.47(3H, m), 7.38(2H, |
| | | | m), 7.24(2H, m), |
| | | | 4.71(2H, s). |
| 7 | 2-[1,3-benzothiazol-2- | 437, 439 | (400MHz, DMSO) δ |
| | ylmethylthio]-3-(4- | | 4.91(2H, s), 7.39-7.43(1H, |
| | chlorophenyl)pyrido[3,2- | | m), 7.47-7.51(1H, m), |
| | d]pyrimidin-4(3H)-one | | 7.59(2H, d, J11.4), |
| | | | 7.68(2H, d, J11.5), 7.87(1H, |
| | | | dd, J4.3, 8.2), 7.95(1H, |
| | | | dd, J8.2, 1.2), 8.03(1H, |
| | | | dd, J1.7, 8.4), 8.11(1H, |
| | | | dd, J1.6, 8.2), 8.78(1H, |
| | | | dd, J1.6, 4.3). |
| 8 | 3-(4-chlorophenyl)-2-[2- | 408, 410 | (400MHz, DMSO) δ |
| | oxo-2-phenylethylthio] | | 4.79(2H, s), 7.54(1H, dd, |
| | pyrido[3,2-d] | | J1.6, 8.2), 7.58-7.62(4H, |
| | pyrimidin-4(3H)-one | | m), 7.69-7.73(4H, m), |
| | | | 8.07-8.09(2H, d, m), |
| | | | 8.70(1H, dd, J1.6, 4.3). |
| 9 | 3-(4-chlorophenyl)-2-{2- | 442, 444 | (400MHz, CDCl₃) δ |
| | (3-chlorophenyl)-2- | | 4.54(2H, s), 7.35(2H, d, |
| | oxoethylthio}pyrido[3,2- | | J11.4), 7.50(1H, t, J7.8), |
| | d]pyrimidin-4(3H)-one | | 7.55-7.59(4H, m), |
| | | | 7.62-7.64(1H, m), |
| | | | 7.93-7.95(1H, m), 8.05-8.06(1H, |
| | | | m), 8.75-8.77(1H, m). |
| 10 | 3-(4-chlorophenyl)-2-(2- | 492, 494 | (400MHz, CDCl₃) δ |
| | oxo-2-[4-trifluoromethoxyphenyl] | | 4.56(2H, s), 7.33-7.39(4H, |
| | ethylthio)pyrido | | m), 7.51-7.58(4H, m), |
| | [3,2-d]pyrimidin-4(3H)- | | 8.13(2H, d, J8.9), 8.76(1H, |
| | one | | dd, J1.9, 4.3). |
| 11 | 3-(4-chlorophenyl)-2-(2- | 476, 478 | (400MHz, CDCl₃) δ |
| | oxo-2-[4-trifluoromethylphenyl] | | 4.58(2H, s), 7.36(2H, d, |
| | ethylthio)pyrido | | J11.6), 7.48-7.50(1H, |
| | [3,2-d]pyrimidin-4(3H)- | | m), 7.54-7.59(3H, m), |
| | one | | 7.82(2H, d, J8.2), 8.18(2H, |
| | | | d, J8.2), 8.77(1H, dd, |
| | | | J1.6, 4.3). |
| 12 | 3-(4-chlorophenyl)-2-{2- | 477, 479 | (400MHz, DMSO) δ |
| | oxo-2-(4-pyrrolidin-1-yl | | 1.97-2.00(4H, m), |
| | phenyl)ethylthio}pyrido | | 3.33-3.41(4H, m), 4.70(2H, s), |
| | [3,2-d]pyrimidin-4(3H)- | | 6.61(2H, d, J9.0), 7.58(2H, |
| | one | | d, J9.0), 7.71(2H, d, |
| | | | J8.6), 7.77(2H, d, J3.1), |
| | | | 7.89(2H, d, J9.0), |
| | | | 8.70-8.72(1H, m). |
| 13 | 3-(4-chlorophenyl)-2-[2- | 409, 411 | 1H(400MHz, CDCl₃) δ |
| | oxo-2-pyridin-2-ylethylthio] | | 4.87(2H, s), 7.35-7.40(3H, |
| | pyrido[3,2-d] | | m), 7.51(1H, dd, J4.3, |
| | pyrimidin-4(3H)-one | | 8.2), 7.54-7.59(3H, m), |
| | | | 7.91(1H, td, J7.8, 1.6), |
| | | | 8.07(1H, dd, J1.2, 7.8), |
| | | | 8.74(1H, dd, J1.6, 4.3), |
| | | | 8.76-8.77(1H, m). |
| 14 | 3-(4-chlorophenyl)-2-[4- | 398, 400 | (400MHz, DMSO) δ |
| | fluorobenzylthio]pyrido | | 4.42(2H, s), 7.09-7.11(2H, |
| | [3,2-d]pyrimidin-4(3H)- | | m), 7.48-7.55(4H, m), |
| | one | | 7.64(2H, d, J8.6), 7.85(1H, |
| | | | dd, J4.3, 8.6), 8.13(1H, |
| | | | dd, J1.6, 8.2), 8.75(1H, |
| | | | dd, J1.6, 4.3). |
| 15 | 2-[3-chlorobenzylthio]-3- | 414, 416 | (400MHz, DMSO) d |
| | (4-chlorophenyl)pyrido | | 4.43(2H, s), 7.28-7.34(2H, |
| | [3,2-d]pyrimidin-4(3H)- | | m), 7.42-7.44(1H, m), |
| | one | | 7.54-7.56(3H, m), |
| | | | 7.64(2H, d, J8.6), 7.86(1H, |
| | | | dd, J4.3, 8.2), 8.13(1H, |
| | | | dd, J1.6, 8.2), 8.75(1H, |
| | | | dd, J1.4, 4.5). |
| 16 | 3-(4-chlorophenyl)-2- | 381, 383 | (400MHz, DMSO) d |
| | [pyridin-2-ylmethylthio] | | 4.55(2H, s), 7.24-7.28(1H, |
| | pyrido[3,2-d]pyrimidin- | | m), 7.54-7.59(3H, m), |
| | 4(3H)-one | | 7.66(2H, d, J9.0), |
| | | | 7.72-7.76(1H, m), 7.84(1H, |
| | | | dd, J4.3, 8.2), 8.09(1H, |
| | | | dd, J1.6, 8.2), 8.47-8.49(1H, |
| | | | m), 8.75(1H, dd, J1.6, |
| | | | 4.3). |
| 17 | 3-(4-chlorophenyl)-2-{5- | 448, 450 | (400MHz, DMSO) d |
| | phenyl-1,2,4-oxadiazol-3- | | 4.67(2H, s), 7.58-7.73(7H, |
| | ylmethylthio}pyrido[3,2- | | m), 7.84(1H, dd, J4.3, |
| | d]pyrimidin-4(3H)-one | | 8.6), 8.04(1H, dd, J1.6, |
| | | | 8.6), 8.07-8.09(2H, m), |
| | | | 8.76(1H, dd, J1.6, 4.3). |
| 18 | 2-{3-(4-chlorophenyl)-4- | 428, 430 | (400MHz, DMSO) d |
| | oxo-3,4- | | 2.35(3H, s), 4.11(2H, s), |
| | dihydropyrido[3,2- | | 6.56(1H, s), 7.59(2H, d, |
| | d]pyrimidin-2-ylthio}-N- | | J8.6), 7.69(2H, d, J8.6), |
| | (5-methylisoxazol-3- | | 7.81(1H, dd, J4.3, 8.2), |
| | yl)acetamide | | 7.87(1H, dd, J1.6, 8.2), |
| | | | 8.73(1H, dd, J1.8, 4.1), |
| | | | 11.27(1H, s). |
| 19 | 3-(4-chlorophenyl)-2-[3- | 403, 405 | (400MHz, CDCl₃) δ |
| | fluorobenzylthio]thieno | | 7.49(2H, m), 7.43(1H, d, |
| | [2,3-d]pyrimidin-4(3H)- | | J6.0), 7.23(3H, m), |
| | one | | 7.14(1H, d, J6.0), 7.10(1H, |
| | | | m), 7.07(1H, m), |
| | | | 6.94(1H, m), 4.35(2H, s). |
| 20 | 3-(4-chlorophenyl)-2-[3- | 403, 405 | (500MHz, DMSO) d |
| | fluorobenzylthio]thieno | | 8.24(1H, d, J5.3), 7.63(2H, d, |
| | [3,2-d]pyrimidin-4(3H)- | | J8.7), 7.52(2H, d, J8.7), |
| | one | | 7.45(1H, d, J5.3), |
| | | | 7.36-7.31(1H, m), |
| | | | 7.30-7.24(2H, m), 7.10-7.03(1H, |
| | | | m), 4.40(2H, s). |
| 21 | 3-(4-chlorophenyl)-2-{2- | 447, 448 | (500MHz, CDCl₃) d |
| | (4-chlorophenyl)-2- | | 8.01(2H, d, J8.3), 7.72(1H, d, |
| | oxoethylthio}thieno | | J5.3), 7.54(2H, d, J8.5), |
| | [3,2-d]pyrimidin-4(3H)- | | 7.50(2H, d, J8.4), |
| | one | | 7.32(2H, d, J8.4), 6.94(1H, d, |
| | | | J5.2), 4.52(2H, s). |
| 22 | 6-(4-chlorophenyl)-5-[3- | 404, 406 | (400MHz, DMSO) δ |
| | fluorobenzylthio][1,3] | | 9.11(1H, s), 7.65(2H, d, |
| | thiazolo[5,4-d]pyrimidin- | | J8.6), 7.53(2H, d, J8.6), |
| | 7(6H)-one | | 7.38-7.30(1H, m), |
| | | | 7.30-7.19(2H, m), |
| | | | 7.11-7.03(1H, m), 4.41(2H, s). |
| 23 | 6-(4-chlorophenyl)-5-{2- | 448, 450 | (400MHz, DMSO) δ |
| | (4-chlorophenyl)-2- | | 9.04(1H, s), 8.05(2H, d, |
| | oxoethylthio}[1,3]thiazolo | | J8.6), 7.71(2H, d, J8.6), |
| | [5,4-d]pyrimidin-7(6H)- | | 7.65(2H, d, J8.6), |
| | one | | 7.58(2H, d, J8.6), 4.77(2H, |
| | | | s). |
| 24 | 6-(4-chlorophenyl)-5-{2- | 448, 450 | (400MHz, DMSO) d |
| | (4-chlorophenyl)-2- | | 4.84(2H, s), 7.59(2H, d, |
| | oxoethylthio}[1,3]thiazolo | | J8.7), 7.65(2H, d, J8.6), |
| | [4,5-d]pyrimidin-7(6H)- | | 7.71(2H, d, J8.8), 8.07(2H, |
| | one | | d, J8.6), 9.57(1H, s). |
| 25 | 2-{5-chloro-1-benzothien- | 473, 475 | (500MHz, CDCl₃) d |
| | 3-ylmethylthio}-1-(4- | | 7.80(1H, d, J2.0), 7.26(1H, d, |
| | chlorophenyl)-9-methyl- | | J8.6), 7.68(1H, s), |
| | 1,9-dihydro-6H-purin-6- | | 7.47(3H, m), 7.34(1H, dd, |
| | one | | J8.6, 2.0), 7.20(2H, d, |
| | | | J8.6), 4.60(2H, s), |
| | | | 3.82(3H, s). |
| 26 | 1-(4-chlorophenyl)-9- | 465, 467 | (400MHz, CDCl₃) d |
| | methyl-2-(2-[4- | | 7.68(1H, s), 7.57(2H, d, |
| | trifluoromethylphenyl] | | J8.1), 7.50(2H, d, J8.5), |
| | ethylthio)-1,9-dihydro- | | 7.33(2H, d, J7.8), |
| | 6H-purin-6-one | | 7.20(2H, d, J8.5), 3.81(3H, |
| | | | s), 3.66(2H, t, J7.8), |
| | | | 3.07(2H, t, J7.8). |
| 27 | 1-(4-chlorophenyl)-2-{2- | 431, 433 | (400MHz, DMSO) δ |
| | (4-chlorophenyl)ethylthio}- | | 2.96(2H, t, J7.6), 3.33(2H, t, |
| | 9-methyl-1,9- | | J7.6), 3.78(3H, s), |
| | dihydro-6H-purin-6-one | | 7.28(2H, d, J8.2), 7.35(2H, |
| | | | d, J8.2), 7.42(2H, d, |
| | | | J9.0), 7.63(2H, d, J8.6), |
| | | | 8.03(1H, s). |
| 28 | 1-(4-chlorophenyl)-2-{2- | 445, 447 | (500MHz, CDCl₃) d |
| | (4-chlorophenyl)-2- | | 8.00(2H, d, J8.7), 7.57(1H, |
| | oxoethylthio}-9-methyl- | | s), 7.52(4H, m), 7.29(2H, |
| | 1,9-dihydro-6H-purin-6- | | d, 8.7), 4.48(2H, s), |
| | one | | 3.37(3H, s). |
| 29 | 1-(4-chlorophenyl)-2-[3- | 401, 403 | (400MHz, CDCl₃) δ |
| | fluorobenzylthio]-9- | | 7.66(1H, s), 7.49(2H, d, |
| | methyl-1,9-dihydro-6H- | | J8.6), 7.26(1H, m) |
| | purin-6-one | | 7.21(2H, d, 8.6), 7.12(2H, m), |
| | | | 6.96(1H, m), 4.33(2H, s), |
| | | | 3.82(3H, s). |
| 30 | 1-(4-chlorophenyl)-2-[3- | 387, 389 | (400MHz, DMSO) δ |
| | fluorobenzylthio]-1,9- | | 13.55 and 13.35(1H, |
| | dihydro-6H-purin-6-one | | brs), 8.23 and 8.03(1H, |
| | | | brs), 7.62(2H, m), |
| | | | 7.48(2H, m), 7.33(1H, m), |
| | | | 7.30(2H, m), 7.07(1H, |
| | | | m), 4.39(2H, s). |
| 31 | 2-{2-(4-chlorophenyl)-2- | 476, 478 | (400MHz, CDCl₃) δ |
| | oxoethylthio}-3-[4- | | 8.78(1H, dd, J3.9, 2.0), |
| | trifluoromethylphenyl] | | 8.01(2H, d, J8.6), 7.87(2H, |
| | pyrido[3,2-d]pyrimidin- | | dd, J8.2), 7.60-7.52(6H, |
| | 4(3H)-one | | m), 4.57(2H, s). |
| 32 | 2-[3-fluorobenzylthio]-3- | 432 | (400MHz, CDCl₃) δ |
| | [4-trifluoromethylphenyl] | | 8.83(1H, dd, J4.5, 1.4), |
| | pyrido[3,2-d] | | 8.02(1H, dd, J8.2, 1.6), |
| | pyrimidin-4(3H)-one | | 7.81(2H, dd, J8.2), 7.71(1H, |
| | | | dd, J8.4, 4.5), 7.47(2H, |
| | | | d, J8.2), 7.27(1H, m), |
| | | | 7.15-7.09(2H, m), |
| | | | 6.98-6.93(1H, m), 4.41(2H, s). |
| 33 | 2-(methylthio)-3-pyridin- | 271 | (360MHz, DMSO) d |
| | 3-ylpyrido[3,2- | | 3.40(3H, s), 7.65(1H, m), |
| | d]pyrimidin-4(3H)-one | | 7.84(1H, m), 8.02(2H, |
| | | | m), 8.70(1H, d, J1.8), |
| | | | 8.75(2H, m). |
| 34 | 1-(4-chlorophenyl)-2-[2- | 403, 405 | (360MHz; CDCl₃) δ |
| | cyclohexylethylthio]-9- | | 7.65(1H, s), 7.50(2H, t, |
| | methyl-1,9-dihydro-6H- | | J4.3), 7.21(2H, t, J4.3), |
| | purin-6-one | | 3.79(3H, s), 3.13(2H, dd, |
| | | | J7.7 and 9.5), 1.72(5H, |
| | | | t, J13.4), 1.57(4H, m), |
| | | | 1.29-1.17(2H, m), |
| | | | 0.97-0.89(2H, m). |
| 35 | 1-(4-chlorophenyl)-2-[2- | 417, 419 | (500MHz; CD₃OD) δ |
| | cyclohexylethylthio]-9- | | 7.99(1H, s), 7.57(2H, d, |
| | ethyl-1,9-dihydro-6H- | | J8.6), 7.32(2H, d, J8.6), |
| | purin-6-one | | 4.29(2H, q, J7.3), |
| | | | 3.19(2H, dd, J7.7 and 9.7), |
| | | | 1.79-1.59(7H, m), |
| | | | 1.54(3H, t, J7.3), |
| | | | 1.41-1.13(4H, m), 1.01-0.93(2H, |
| | | | m). |
| 36 | 1-(4-chlorophenyl)-9- | 403, 405 | (500MHz; CD₃OD) δ |
| | ethyl-2[3,3,3- | | 8.02(1H, s), 7.58(2H, d, |
| | trifluoropropylthio]-1,9- | | J8.6), 7.36(2H, d, J8.6), |
| | dihydro-6H-purin-6-one | | 4.29(2H, q, J7.3), |
| | | | 3.35(2H, dd, J7.8 and 10.5), |
| | | | 2.72-2.64(2H, m), |
| | | | 1.53(3H, t, J7.3). |
| 37 | 1-(4-chlorophenyl)-9- | 349, 351 | (400MHz; CD₃OD) δ |
| | ethyl-2-propylthio-1,9- | | 7.99(1H, s), 7.59-7.55(2H, |
| | dihydro-6H-purin-6-one | | m), 7.35-7.31(2H, m), |
| | | | 4.28(2H, q, J7.3), |
| | | | 3.16(2H, t, J7.3), |
| | | | 1.79-1.71(2H, m), 1.54(3H, t, |
| | | | J7.3), 1.01(3H, t, J7.4). |
| 38 | 1-(4-chlorophenyl)-2- | 361, 363 | (400MHz; CD₃OD) δ |
| | [cyclopropylmethylthio]- | | 7.99(1H, s), 7.58(2H, d, |
| | 9-ethyl-1,9-dihydro-6H- | | J8.6), 7.34(2H, d, J8.6), |
| | purin-6-one | | 4.28(2H, q, J7.3), |
| | | | 3.14(2H, d, J7.3), 1.54(3H, t, |
| | | | J7.3), 1.22-1.12(1H, m), |
| | | | 0.61-0.55(2H, m), |
| | | | 0.31-0.28(2H, m). |
| 39 | 1-(4-chlorophenyl)-9- | 389, 391 | (500MHz; CD₃OD) δ |
| | methyl-2-[3,3,3- | | 7.96(1H, s), 7.58(2H, d, |
| | trifluoropropylthio]-1,9- | | J8.7), 7.35(2H, d, J8.6), |
| | dihydro-6H-purin-6-one | | 3.84(3H, s), 3.36(2H, m), |
| | | | 2.73-2.63(2H, m). |
| 40 | 1-(4-chlorophenyl)-9- | 415, 417 | (500MHz; CD₃OD) δ |
| | cyclopropyl-2-[3,3,3- | | 7.99(1H, s), 7.58(2H, d, |
| | trifluoropropylthio]-1,9- | | J8.6), 7.35(2H, d, J8.6), |
| | dihydro-6H-purin-6-one | | 3.53-3.49(1H, m), |
| | | | 3.37(2H, m), 2.77-2.67(2H, |
| | | | m), 1.17(4H, d, J5.5). |
| 41 | 1-(4-chlorophenyl)-9- | 389, 391 | (400MHz; DMSO) δ |
| | ethyl-2-[2,2,2- | | 8.13(1H, s), 7.67(2H, d, |
| | trifluoroethylthio]-1,9- | | J8.6), 7.51(2H, d, J8.6), |
| | dihydro-6H-purin-6-one | | 4.30-4.20(4H, m), |
| | | | 1.43(3H, t, J7.2). |
| 42 | 1-(4-chlorophenyl)-9- | 417, 419 | (360MHz; DMSO) δ |
| | ethyl-2-[4,4,4- | | 8.09(1H, s), 7.63(2H, d, |
| | trifluorobutylthio]-1,9- | | J8.6), 7.46(2H, d, J8.6), |
| | dihydro-6H-purin-6-one | | 4.20(2H, q, J7.2), |
| | | | 3.17(2H, t, J7.2), |
| | | | 2.43-2.29(2H, m), 1.96-1.88(2H, |
| | | | m), 1.43(3H, t, J7.2). |
| 43 | 1-(4-chlorophenyl)-9- | 527, 529 | (500MHz; DMSO) δ |
| | phenyl-2-(2-[4- | | 8.46(1H, s), 7.84(2H, d, |
| | trifluoromethylphenyl] | | J7.3), 7.65(2H, d, J8.6), |
| | ethylthio)-1,9-dihydro- | | 7.60-7.53(5H, m), |
| | 6H-purin-6-one | | 7.48(2H, d, J8.5), 7.25(2H, d, |
| | | | J7.9), 3.27(2H, m) |
| | | | 2.98(2H, t, J7.8). |
| 44 | 1-(4-chlorophenyl)-2- | 369, 371 | (500MHz; DMSO) δ |
| | methylthio-9-phenyl-1,9- | | 8.49(1H, s), 7.89(2H, d, |
| | dihydro-6H-purin-6-one | | J7.5), 7.67-7.61(4H, m), |
| | | | 7.49(3H, t, J7.5), |
| | | | 2.44(3H, s). |
| 45 | 1-(4-chlorophenyl)-9- | 451, 453 | (500MHz; CD₃OD) δ |
| | phenyl-2-[3,3,3- | | 8.32(1H, s), 7.79(2H, d, |
| | trifluoropropylthiol]-1,9- | | J7.6), 7.60-7.58(4H, m), |
| | dihydro-6H-purin-6-one | | 7.52(1H, t, J7.4), |
| | | | 7.39(2H, d, J8.6), 3.25(2H, |
| | | | m), 2.65-2.55(2H, m). |
| 46 | 1-(4-chlorophenyl)-9- | 465, 467 | (500MHz; CD₃OD) δ |
| | phenyl-2-[4,4,4- | | 8.30(1H, s), 7.77(2H, d, |
| | trifluorobutylthio]-1,9- | | J7.6), 7.63-7.59(4H, m), |
| | dihydro-6H-purin-6-one | | 7.54(1H, t, J7.4), |
| | | | 7.39(2H, d, J8.6), 3.12(2H, t, |
| | | | J7.4), 2.19-2.09(2H, m), |
| | | | 1.96-1.90(2H, m). |
| 47 | 4-{9-methyl-6-oxo-2- | 380 | (500MHz; CD₃OD) δ |
| | [3,3,3-trifluoropropylthio]- | | 7.98(1H, s), 7.96(2H, d, |
| | 6,9-dihydro-1H- | | J8.4), 7.59(2H, d, J8.4), |
| | purin-1-yl}benzonitrile | | 3.85(3H, s), 3.39(2H, dd, |
| | | | J7.7, 10.1), |
| | | | 2.73-2.65(2H, m). |
| 48 | 9-methyl-1-(4- | 369 | (360MHz; CD₃OD) δ |
| | methylphenyl)-2-[3,3,3- | | 7.95(1H, s), 7.38(2H, d, |
| | trifluoropropylthio]-1,9- | | J8.1), 7.19(2H, d, J8.3), |
| | dihydro-6H-purin-6-one | | 3.84(3H, s), 3.35(2H, m), |
| | | | 2.73-2.59(2H, m), |
| | | | 2.44(3H, s). |

### EXAMPLE 49

### 3-(4-Chlorophenyl)-2-(3-oxo-4-phenylpiperazin-1-yl)pyrido [3,2-d]pyrimidin-4(3H)-one

A mixture of Description 6 (50 mg, 0.17 mmol), 1-phenylpiperazin-2-one (Tetrahedron Lett. 1998, 39, 7459) (37 mg, 0.21 mmol) and potassium carbonate (240 mg, 1.7 mmol) in anhydrous acetonitrile (2 ml) was refluxed for 5 h. The reaction was cooled to room temperature and the salts removed by filtration and washed with acetonitrile (3×10 ml). The filtrate was evaporated in vacuo and the resulting residue purified by mass directed HPLC, then passed through a strong cation exchange (SCX) cartridge to give the title compound (8 mg, 10%). ¹H (400 MHz, DMSO) δ 8.65 (1H, dd, J1.6, 4.3), 7.94 (1H, dd, J1.6, 8.2), 7.77 (1H, dd, J4.1, 8.4), 7.64-7.61 (4H, m), 7.40-7.36 (2H, m), 7.26-7.22 (3H, m), 3.86 (1H, s), 3.43 (4H, s). M/z (ES⁺) 432, 434 (M+H⁺).

### EXAMPLE 50

### 3-4-Chlorophenyl-2-{2-4-chlorophenylethylamino}pyrido [3,2-d]pyrimidin-4(3H)-one

A mixture of Description 6 (58 mg, 0.2 mmol) and 2-(4-chlorophenyl)ethylamine (37 mg, 0.24 mmol) and potassium carbonate (138 mg, 1 mmol) in acetonitrile (2 ml) was heated at refluxfor 4 h, then cooled to room temperature. The reaction mixture was then evaporated in vacuo and the residue partitioned between dichloromethane (15 ml) and water (2×15 ml). The organic layer was dried over MgSO₄, filtered and evaporated. The crude product was purified by preparative thin layer chromatography (eluant: 5% methanol in dichloromethane) to give the title compound as a beige solid (20 mg, 24%). ¹H (360 MHz, DMSO) δ 8.43 (1H, dd, J1.4, 4.2), 7.75-7.72 (1H, m), 7.64-7.59 (3H, m), 7.37 (2H, d, J8.6), 7.33 (2H, d, J8.5), 7.21 (2H, d, J8.4), 6.07 (1H, t, J5.8), 3.50-3.45 (2H, m), 2.82 (2H, t, J 7.0). M/z (ES⁺) 411, 413 (M+H⁺).

### EXAMPLE 51

### 3-(4-Chlorophenyl)-2-[3-fluorobenzyloxy]thieno[3,2-d]pyrimidin-4(3H)-one

To 3-fluorobenzylalcohol (16 mg, 0.127 mmol) in THF (1 ml) at 0° C. was added NaH (60% dispersion in oil, 5 mg, 0.130 mmol) and the solution allowed to warm to room temperature for 10 min. A solution of Description 10 (25 mg, 0.084 mmol) in THF (1 ml) was added and the reaction stirred for 18 h at room temperature. The reaction was concentrated, then dissolved in water (2 ml) and dichloromethane (2 ml) and the mixture vortexed. After settling, the mixture was added to a phase separation cartridge and the dichloromethane phase was separated and concentrated. The crude mixture was dissolved in dimethylsulfoxide and purified by mass-directed HPLC to give the title compound as a white solid (10 mg, 30%). ¹H (400 MHz, DMSO) δ 8.20 (1H, d, J4.7), 7.59 (2H, d, J7.7), 7.51 (2H, d, J7.7), 7.35 (2H, m), 7.08 (2H, m), 6.99 (1H, d, J8.8), 5.42 (2H, s). M/z (ES⁺) 387, 389 (M+H⁺).

### EXAMPLE 52

### 3-(4-Chlorophenyl)-2-[3-fluorobenzylamino]thieno[3,2-d]pyrimidin-4(3H)-one

Description 10 (25 mg, 0.084 mmol), 3-fluorobenzylamine (12 mg, 0.105 mmol) and potassium carbonate (35 mg, 0.254 mmol) in acetonitrile (1.5 ml) were heated to reflux for 4 h. The solvent was removed and the reaction then dissolved in water (2 ml) and dichloromethane (2 ml) added and the mixture vortexed. After settling, the mixture was added to a phase separation cartridge and the dichloromethane phase was separated and concentrated. The crude mixture was dissolved in dimethylsulfoxide and purified by mass-directed HPLC to provide the title compound as a white solid (9 mg, 27%). ¹H (400 MHz, DMSO) δ 7.74 (1H, d, J5.3), 7.57 (2H, d, J8.3), 7.29 (3H, m), 7.14 (1H, d, J5.3), 6.95 (3H, m), 4.62 (2H, d, J5.4), 4.47 (1H, brm). M/z (ES⁺) 386, 388 (M+H⁺).

### EXAMPLE 53

### 1-(4-chlorophenyl)-9-ethyl-2-(2-[4-trifluoromethylphenyl]ethyl amino)-1,9-dihydro-6H-purin-6-one

Prepared from Description 26 (75 mg, 0.24 mmol) and 2-[4-(trifluoromethyl)phenyl]ethanamine (WO-A-03080578) (69 mg, 0.37 mmol) according to Example 49. The crude product was purified by preparative thin layer chromatography (eluant: 5% methanol in dichloromethane with 0.1% ammonia) to give the title compound as a white solid (37 mg, 33%). 1H NMR (500 MHz; CD₃OD) δ 7.77 (1H, s), 7.57-7.52 (4H, m), 7.35 (2H, d, J8.0), 7.19 (2H, d, J8.0), 4.17 (2H, q, J7.3), 3.63 (2H, t, J7.0), 2.98 (2H, t, J7.0), 1.51 (3H, t, J 7.3). M/z (ES⁺) 462, 464 (M+H⁺).

### EXAMPLE 54

### 1-(4-chlorophenyl)-9-ethyl-2-(2-[2-fluoro-4-trifluoromethylphenyl]ethylamino)-1,9-dihydro-6H-purin-6-one

Prepared from Description 26 (190 mg, 0.62 mmol) and Description 27 (153 mg, 0.74 mmol) according to Example 49. The crude product was purified by preparative thin layer chromatography (eluant: ethyl acetate with 0.1% ammonia) to give the title compound as a white solid (110 mg, 37%). ¹H NMR (400 MHz; DMSO) δ 7.78 (1H, s), 7.60-7.44 (5H, m), 7.28-7.24 (2H, m), 6.10 (1H, t, J5.7), 4.04 (2H, q, J7.2), 3.49 (2H, q, J6.5), 2.95 (2H, t, J6.8), 1.40 (3H, t, J7.2). M/z (ES⁺) 480, 482 (M+H⁺).

Examples 55-205, as shown below, were prepared using the appropriate purine or pyrimidinone core according to procedures described above. Synthesis of these cores is described above in Descriptions 5, 7, 8, 12, 15, 16, 19-21, 23-25, 28, 29, 47-76. Alkyl iodides, bromides, chlorides and amines are commercially available, prepared by known methods or by the methods described above. Where the product did not precipitate analytically pure from the reaction mixture it was purified by recrystallisation, flash column chromatography, preparative thin layer chromatography or mass directed HPLC as appropriate.

| | | M/z ES+ | Made according |
|---|---|---|---|
| Example | Name | [M + H⁺] | to procedure of |
| 55 | 3-(4-chlorophenyl)-2- | 292, 294 | Example |
| | methylaminothieno[3,2-d]pyrimidin-4(3H)- | | 52 |
| | one | | |
| 56 | 3-(4-chlorophenyl)-2-{2-(2-chlorophenyl)-2- | 442, 444 | Example 2 |
| | oxoethylthio}pyrido[3,2-d]pyrimidin-4(3H)- | | |
| | one | | |
| 57 | 2-{2-(4-chlorophenyl)-2-oxoethylthio}-3- | 408, 410 | Example 2 |
| | phenylpyrido[3,2-d]pyrimidin-4(3H)-one | | |
| 58 | 3-(4-chlorophenyl)-2-[2- | 394, 396 | Example 2 |
| | phenylethylthio]pyrido[3,2-d]pyrimidin- | | |
| | 4(3H)-one | | |
| 59 | 3-(4-chlorophenyl)-2-[2- | 398, 400 | Example 2 |
| | fluorobenzylthio]pyrido[3,2-d]pyrimidin- | | |
| | 4(3H)-one | | |
| 60 | 6-(4-chlorophenyl)-5-{2-(4- | 434, 436 | Example 2 |
| | chlorophenyl)ethylthio}[1,3]thiazolo[5,4- | | |
| | d]pyrimidin-7(6H)-one | | |
| 61 | 3-(4-chlorophenyl)-2-{2-(3- | 428, 430 | Example 2 |
| | chlorophenyl)ethylthio}pyrido[3,2- | | |
| | d]pyrimidin-4(3H)-one | | |
| 62 | 3-(4-chlorophenyl)-2-(2-[4- | 462, 464 | Example 2 |
| | trifluoromethylphenyl]ethylthio)pyrido[3,2- | | |
| | d]pyrimidin-4(3H)-one | | |
| 63 | 6-(4-chlorophenyl)-5-[3- | 404, 406 | Example 2 |
| | fluorobenzylthio][1,3]thiazolo[4,5- | | |
| | d]pyrimidin-7(6H)-one | | |
| 64 | 6-(4-chlorophenyl)-5-{2-(4- | 434, 436 | Example 2 |
| | chlorophenyl)ethylthio}[1,3]thiazolo[4,5- | | |
| | d]pyrimidin-7(6H)-one | | |
| 65 | 2-{6-chloro-1-benzothien-3-ylmethylthio}-1- | 473, 475 | Example 2 |
| | (4-chlorophenyl)-9-methyl-1,9-dihydro-6H- | | |
| | purin-6-one | | |
| 66 | 2-{5-chloro-1-benzothien-3-ylmethylthio}-1- | 487, 489 | Example 2 |
| | (4-chlorophenyl)-9-ethyl-1,9-dihydro-6H- | | |
| | purin-6-one | | |
| 67 | 2-{5-chloro-1-benzothien-3-ylmethylthio}-1- | 501, 503 | Example 2 |
| | (4-chlorophenyl)-9-isopropyl-1,9-dihydro- | | |
| | 6H-purin-6-one | | |
| 68 | 3-(6-chloropyridin-3-yl)-2-[3- | 404, 406 | Example 2 |
| | fluorobenzylthio]thieno[3,2-d]pyrimidin- | | |
| | 4(3H)-one | | |
| 69 | 2-{5-chloro-1-benzothien-3-ylmethylthio}-3- | 491, 493 | Example 2 |
| | [4-trifluoromethylphenyl]pyrido[3,2- | | |
| | d]pyrimidin-4(3H)-one | | |
| 70 | 1-(3-chlorophenyl)-9-methyl-2-(2-[4- | 465, 467 | Example 2 |
| | trifluoromethylphenyl]ethylthio)-1,9- | | |
| | dihydro-6H-purin-6-one | | |
| 71 | 1-(4-chlorophenyl)-2-[3,4- | 451, 453 | Example 2 |
| | dichlorobenzylthio]-9-methyl-1,9-dihydro- | | |
| | 6H-purin-6-one | | |
| 72 | 1-(4-chlorophenyl)-9-cyclopropyl-2-(2-[4- | 491, 493 | Example 2 |
| | trifluoromethylphenyl]ethylthio)-1,9- | | |
| | dihydro-6H-purin-6-one | | |
| 73 | 1-(4-chlorophenyl)-9-ethyl-2-(2-[4- | 479, 481 | Example 2 |
| | trifluoromethylphenyl]ethylthio)-1,9- | | |
| | dihydro-6H-purin-6-one | | |
| 74 | 3-[4-trifluoromethylphenyl]-2-(2-[4- | 501 | Example 2 |
| | trifluoromethylphenyl]ethylthio)thieno[3,2- | | |
| | d]pyrimidin-4(3H)-one | | |
| 75 | 3-[4-trifluoromethylphenyl]-2-(2-[4- | 496 | Example 2 |
| | trifluoromethylphenyl]ethylthio)pyrido[3,2- | | |
| | d]pyrimidin-4(3H)-one | | |
| 76 | 1-(4-chlorophenyl)-2-{2-(2,4- | 466, 468 | Example 2 |
| | dichlorophenyl)ethylthio}-9-methyl-1,9- | | |
| | dihydro-6H-purin-6-one | | |
| 77 | 3-(4-chlorophenyl)-2-[3,4- | 448, 450 | Example 2 |
| | dichlorobenzylthio]pyrido[3,2-d]pyrimidin- | | |
| | 4(3H)-one | | |
| 78 | 2-[3-chloro-4-fluorobenzylthio]-3-(4- | 432, 434 | Example 2 |
| | chlorophenyl)pyrido[3,2-d]pyrimidin-4(3H)- | | |
| | one | | |
| 79 | 1-(4-chlorophenyl)-2-(2-[3-fluoro-4- | 483, 485 | Example 2 |
| | trifluoromethylphenyl]ethylthio)-9-methyl- | | |
| | 1,9-dihydro-6H-purin-6-one | | |
| 80 | 3-(4-fluorophenyl)-2-(2-[4- | 445 | Example 2 |
| | trifluoromethylphenyl]ethylthio)pyrido[3,2- | | |
| | d]pyrimidin-4(3H)-one | | |
| 81 | 1-(4-fluorophenyl)-9-methyl-2-(2-[4- | 449 | Example 2 |
| | trifluoromethylphenyl]ethylthio)-1,9- | | |
| | dihydro-6H-purin-6-one | | |
| 82 | 1-(4-chlorophenyl)-9-methyl-2-(2-[4- | 481, 483 | Example 2 |
| | trifluoromethoxyphenyl]ethylthio)-1,9- | | |
| | dihydro-6H-purin-6-one | | |
| 83 | 1-(4-chlorophenyl)-2-{2-(4- | 415, 417 | Example 2 |
| | fluorophenyl)ethylthio}-9-methyl-1,9- | | |
| | dihydro-6H-purin-6-one | | |
| 84 | 1-(4-chlorophenyl)-2-[2,4- | 451, 453 | Example 2 |
| | dichlorobenzylthio]-9-methyl-1,9-dihydro- | | |
| | 6H-purin-6-one | | |
| 85 | 2-{5-chloro-1-benzothien-3-ylmethylthio}-9- | 507, 509 | Example 2 |
| | methyl-1-[4-trifluoromethylphenyl]-1,9- | | |
| | dihydro-6H-purin-6-one | | |
| 86 | 9-methyl-1-[4-trifluoromethylphenyl]-2-(2- | 499 | Example 2 |
| | [4-trifluoromethylphenyl]ethylthio)-1,9- | | |
| | dihydro-6H-purin-6-one | | |
| 87 | 4-[2-{5-chloro-1-benzothien-3-ylmethylthio}- | 461, 463 | Example 2 |
| | 4-oxopyrido[3,2-d]pyrimidin-3(4H)- | | |
| | yl]benzonitrile | | |
| 88 | ethyl {1-(4-chlorophenyl)-9-methyl-6-oxo- | 379, 381 | Example 2 |
| | 6,9-dihydro-1H-purin-2-ylthio}acetate | | |
| 89 | 4-[4-oxo-2-(2-[4- | 453 | Example 2 |
| | trifluoromethylphenyl]ethylthio)pyrido[3,2- | | |
| | d]pyrimidin-3(4H)-yl]benzonitrile | | |
| 90 | 1-(4-chlorophenyl)-2-[3,4- | 465, 467 | Example 2 |
| | dichlorobenzylthio]-9-ethyl-1,9-dihydro-6H- | | |
| | purin-6-one | | |
| 91 | 1-(4-chlorophenyl)-9-propyl-2-(2-[4- | 493, 495 | Example 2 |
| | trifluoromethylphenyl]ethylthio)-1,9- | | |
| | dihydro-6H-purin-6-one | | |
| 92 | 1-(4-chlorophenyl)-9-methyl-2-(2-[4- | 448, 450 | Example |
| | trifluoromethylphenyl]ethylamino)-1,9- | | 53 |
| | dihydro-6H-purin-6-one | | |
| 93 | 1-(4-chlorophenyl)-2-[3,4- | 479, 481 | Example 2 |
| | dichlorobenzylthio]-9-propyl-1,9-dihydro- | | |
| | 6H-purin-6-one | | |
| 94 | 2-{5-chloro-1-benzothien-3-ylmethylthio}-9- | 523, 525 | Example 2 |
| | methyl-1-[4-trifluoromethoxyphenyl]-1,9- | | |
| | dihydro-6H-purin-6-one | | |
| 95 | 2-{5-chloro-1-benzothien-3-ylmethylthio}-1- | 513, 515 | Example 2 |
| | (4-chlorophenyl)-9-(cyclopropylmethyl)-1,9- | | |
| | dihydro-6H-purin-6-one | | |
| 96 | 1-(4-chlorophenyl)-2-{2-(4-chlorophenyl)-2- | 485, 487 | Example 2 |
| | oxoethylthio}-9-(cyclopropylmethyl)-1,9- | | |
| | dihydro-6H-purin-6-one | | |
| 97 | 1-(4-chlorophenyl)-9-cyclopropyl-2-[3- | 427, 429 | Example 2 |
| | fluorobenzylthio]-1,9-dihydro-6H-purin-6- | | |
| | one | | |
| 98 | 2-[3-chloro-4-fluorobenzylthio]-1-(4- | 461, 463 | Example 2 |
| | chlorophenyl)-9-cyclopropyl-1,9-dihydro- | | |
| | 6H-purin-6-one | | |
| 99 | 1-(4-chlorophenyl)-9-cyclopropyl-2-[3,4- | 477, 479 | Example 2 |
| | dichlorobenzylthio]-1,9-dihydro-6H-purin- | 481 | |
| | 6-one | | |
| 100 | 1-(4-chlorophenyl)-9-cyclopropyl-2-{3- | 477, 479 | Example 2 |
| | trifluoromethylbenzylthio}-1,9-dihydro-6H- | | |
| | purin-6-one | | |
| 101 | 2-[3-chlorobenzylthio]-1-(4-chlorophenyl)-9- | 443, 445 | Example 2 |
| | cyclopropyl-1,9-dihydro-6H-purin-6-one | | |
| 102 | 2-{5-chloro-1-benzothien-3-ylmethylthio}-1- | 499, 501 | Example 2 |
| | (4-chlorophenyl)-9-cyclopropyl-1,9-dihydro- | | |
| | 6H-purin-6-one | | |
| 103 | 1-(4-chlorophenyl)-2-{2-(4-chlorophenyl)-2- | 471, 473 | Example 2 |
| | oxoethylthio}-9-cyclopropyl-1,9-dihydro-6H- | | |
| | purin-6-one | | |
| 104 | 1-(4-chlorophenyl)-9-cyclopropyl-2-(2-oxo-2- | 505, 507 | Example 2 |
| | [4-trifluoromethylphenyl]ethylthio)-1,9- | | |
| | dihydro-6H-purin-6-one | | |
| 105 | 1-(4-chlorophenyl)-9-cyclopropyl-2-{2-(4- | 455, 457 | Example 2 |
| | fluorophenyl)-2-oxoethylthio}-1,9-dihydro- | | |
| | 6H-purin-6-one | | |
| 106 | 1-(4-chlorophenyl)-9-cyclopropyl-2-[2,4- | 477, 479 | Example 2 |
| | dichlorobenzylthio]-1,9-dihydro-6H-purin- | | |
| | 6-one | | |
| 107 | 1-(4-chlorophenyl)-9-cyclopropyl-2-{2-(2,4- | 491, 493 | Example 2 |
| | dichlorophenyl)ethylthio}-1,9-dihydro-6H- | | |
| | purin-6-one | | |
| 108 | 3-(4-chlorophenyl)-2-[3-fluorobenzylthio]-7- | 417, 419 | Example 2 |
| | methylthieno[3,2-d]pyrimidin-4(3H)-one | | |
| 109 | 2-{5-chloro-1-benzothien-3-ylmethylthio}-3- | 489, 491 | Example 2 |
| | (4-chlorophenyl)-7-methylthieno[3,2- | | |
| | d]pyrimidin-4(3H)-one | | |
| 110 | 4-[2-[3-fluorobenzylthio]-4-oxopyrido[3,2- | 389 | Example 2 |
| | d]pyrimidin-3(4H)-yl]benzonitrile | | |
| 111 | 1-(4-chlorophenyl)-9-cyclopropyl-2-(2-[2- | 507, 509 | Example 2 |
| | hydroxy-4- | | |
| | trifluoromethylphenyl]ethylthio)-1,9- | | |
| | dihydro-6H-purin-6-one | | |
| 112 | 2-{5-chloro-1-benzothien-3-ylmethylthio}-9- | 467, 469 | Example 2 |
| | ethyl-1-(4-methylphenyl)-1,9-dihydro-6H- | | |
| | purin-6-one | | |
| 113 | 2-{5-chloro-1-benzothien-3-ylmethylthio}-1- | 501, 503 | Example 2 |
| | (4-chlorophenyl)-9-propyl-1,9-dihydro-6H- | | |
| | purin-6-one | | |
| 114 | 1-(4-chlorophenyl)-9-cyclopropyl-2-(2-[2- | 509, 511 | Example 2 |
| | fluoro-4-trifluoromethylphenyl]ethylthio)- | | |
| | 1,9-dihydro-6H-purin-6-one | | |
| 115 | 1-(4-bromophenyl)-2-{5-chloro-1- | 531, 533, | Example 2 |
| | benzothien-3-ylmethylthio}-9-ethyl-1,9- | 535 | |
| | dihydro-6H-purin-6-one | | |
| 116 | 2-[1,3-benzothiazol-2-ylmethylthio]-1-(4- | 466, 468 | Example 2 |
| | chlorophenyl)-9-cyclopropyl-1,9-dihydro- | | |
| | 6H-purin-6-one | | |
| 117 | 1-(4-chlorophenyl)-9-cyclopropyl-2-{2- | 495, 497 | Example 2 |
| | fluoro-4-trifluoromethylbenzylthio}-1,9- | | |
| | dihydro-6H-purin-6-one | | |
| 118 | 1-(4-chlorophenyl)-9-cyclopropyl-2-{2- | 495, 497 | Example 2 |
| | fluoro-5-trifluoromethylbenzylthio}-1,9- | | |
| | dihydro-6H-purin-6-one | | |
| 119 | 1-(4-chlorophenyl)-9-cyclopropyl-2-{3- | 495, 497 | Example 2 |
| | fluoro-4-trifluoromethylbenzylthio}-1,9- | | |
| | dihydro-6H-purin-6-one | | |
| 120 | 1-(4-chlorophenyl)-9-cyclopropyl-2-(5- | 534, 536 | Example 2 |
| | trifluoromethyl-1,3-benzothiazol-2- | | |
| | ylmethylthio)-1,9-dihydro-6H-purin-6-one | | |
| 121 | 2-{5-chloro-1-benzothien-3-ylmethylthio}-1- | 497, 499 | Example 2 |
| | [4-dimethylaminophenyl]-9-ethyl-1,9- | | |
| | dihydro-6H-purin-6-one | | |
| 122 | 3-(4-chlorophenyl)-2-[3,4- | 467, 469, | Example 2 |
| | dichlorobenzylthio]-7-methylthieno[3,2- | | |
| | d]pyrimidin-4(3H)-one | | |
| 123 | 2-{2-(2,4-dichlorophenyl)ethylthio}-1-(4- | 449, 451 | Example 2 |
| | fluorophenyl-9-methyl-1,9-dihydro-6H- | | |
| | purin-6-one | | |
| 124 | 1-(4-chlorophenyl)-2-{2-(2,4- | 479, 481 | Example 2 |
| | dichlorophenyl)ethylthio}-9-ethyl-1,9- | | |
| | dihydro-6H-purin-6-one | | |
| 125 | 1-(4-chlorophenyl)-9-ethyl-2-pentylthio-1,9- | 377, 379 | Example 2 |
| | dihydro-6H-purin-6-one | | |
| 126 | 1-(4-chlorophenyl)-9-ethyl-2-[3- | 377, 379 | Example 2 |
| | methylbutylthio]-1,9-dihydro-6H-purin-6- | | |
| | one | | |
| 127 | 1-(4-chlorophenyl)-2-[2- | 400, 402 | Example |
| | cyclohexylethylamino]-9-ethyl-1,9-dihydro- | | 53 |
| | 6H-purin-6-one | | |
| 128 | 1-(4-chlorophenyl)-2-(2-[2-chloro-4- | 499, 501 | Example 2 |
| | trifluoromethylphenyl]ethylthio)-9-methyl- | | |
| | 1,9-dihydro-6H-purin-6-one | | |
| 129 | 1-(4-chlorophenyl)-2-(2-[2-chloro-4- | 513, 515 | Example 2 |
| | trifluoromethylphenyl]ethylthio)-9-ethyl- | | |
| | 1,9-dihydro-6H-purin-6-one | | |
| 130 | 2-{2-(2,4-dichlorophenyl)ethylthio}-9-ethyl- | 463, 465 | Example 2 |
| | 1-(4-fluorophenyl)-1,9-dihydro-6H-purin-6- | | |
| | one | | |
| 131 | 9-ethyl-1-(4-fluorophenyl)-2-(2-[2-fluoro-4- | 481 | Example 2 |
| | trifluoromethylphenyl]ethylthio)-1,9- | | |
| | dihydro-6H-purin-6-one | | |
| 132 | 2-(2-[2-chloro-4- | 497, 499 | Example 2 |
| | trifluoromethylphenyl]ethylthio)-9-ethyl-1- | | |
| | (4-fluorophenyl)-1,9-dihydro-6H-purin-6- | | |
| | one | | |
| 133 | 2-{5-chloro-1-benzothien-3-ylmethylamino}- | 470, 472 | Example |
| | 1-(4-chlorophenyl)-9-ethyl-1,9-dihydro-6H- | | 53 |
| | purin-6-one | | |
| 134 | 1-(4-chlorophenyl)-9-ethyl-2-[3- | 398, 400 | Example |
| | fluorobenzylamino]-1,9-dihydro-6H-purin- | | 53 |
| | 6-one | | |
| 135 | 2-{5-chloro-1,3-benzothiazol-2- | 488, 490 | Example 2 |
| | ylmethylthio}-1-(4-chlorophenyl)-9-ethyl- | | |
| | 1,9-dihydro-6H-purin-6-one | | |
| 136 | 1-(2,4-dichlorophenyl)-2-{2-(2,4- | 500, 502 | Example 2 |
| | dichlorophenyl)ethylthio}-9-methyl-1,9- | | |
| | dihydro-6H-purin-6-one | | |
| 137 | 1-(4-chlorophenyl)-2-(2-[2-fluoro-4- | 483, 485 | Example 2 |
| | trifluoromethylphenyl]ethylthio)-9-methyl- | | |
| | 1,9-dihydro-6H-purin-6-one | | |
| 138 | 1-(4-chlorophenyl)-9-ethyl-2-(2-[2-fluoro-4- | 497, 499 | Example 2 |
| | trifluoromethylphenyl]ethylthio)-1,9- | | |
| | dihydro-6H-purin-6-one | | |
| 139 | 1-(4-chlorophenyl)-2-{2-(2,4- | 479, 481 | Example 2 |
| | dichlorophenyl)-2-oxoethylthio}-9-methyl- | | |
| | 1,9-dihydro-6H-purin-6-one | | |
| 140 | 9-ethyl-1-(4-fluorophenyl)-2-[3,3,3- | 387, 389 | Example 2 |
| | trifluoropropylthio]-1,9-dihydro-6H-purin- | | |
| | 6-one | | |
| 141 | 1-(4-chlorophenyl)-9-ethyl-2-[3,3,3- | 386, 388 | Example |
| | trifluoropropylamino]-1,9-dihydro-6H- | | 53 |
| | purin-6-one | | |
| 142 | 9-cyclopropyl-2-{2-(2,4- | 475, 477 | Example 2 |
| | dichlorophenyl)ethylthio}-1-(4- | | |
| | fluorophenyl)-1,9-dihydro-6H-purin-6-one | | |
| 143 | 1-(4-chlorophenyl)-2-{2-(2,4- | 495, 497 | Example 2 |
| | dichlorophenyl)ethylthio}-9-(2- | | |
| | hydroxyethyl)-1,9-dihydro-6H-purin-6-one | | |
| 144 | 9-cyclopropyl-1-(4-fluorophenyl)-2-(3,3,3- | 399 | Example 2 |
| | trifluoropropylthio]-1,9-dihydro-6H-purin- | | |
| | 6-one | | |
| 145 | 1-(4-chlorophenyl)-2-{2-(2,4- | 462, 464 | Example |
| | dichlorophenyl)ethylamino}-9-ethyl-1,9- | | 53 |
| | dihydro-6H-purin-6-one | | |
| 146 | 1-(4-chlorophenyl)-2- | 375, 377 | Example 2 |
| | [cyclobutylmethylthio]-9-ethyl-1,9-dihydro- | | |
| | 6H-purin-6-one | | |
| 147 | 9-cyclopropyl-1-(4-fluorophenyl)-2-(2-[2- | 493 | Example 2 |
| | fluoro-4-trifluoromethylphenyl]ethylthio)- | | |
| | 1,9-dihydro-6H-purin-6-one | | |
| 148 | 1-(4-chlorophenyl)-9-ethyl-2-[3,3,3-trifluoro- | 419, 421 | Example 2 |
| | 2-hydroxypropylthio]-1,9-dihydro-6H-purin- | | |
| | 6-one | | |
| 149 | 1-(4-chlorophenyl)-9-ethyl-2-[3,3,3-trifluoro- | 435, 437 | Example 2 |
| | 2,2-dihydroxypropylthio]-1,9-dihydro-6H- | | |
| | purin-6-one | | |
| 150 | 1-(4-chlorophenyl)-2-[2- | 403, 405 | Example 2 |
| | cyclopentylethylthio]-9-ethyl-1,9-dihydro- | | |
| | 6H-purin-6-one | | |
| 151 | 1-(4-chlorophenyl)-9-ethyl-2-{2-methyl-1,3- | 418, 420 | Example 2 |
| | thiazol-4-ylmethylthio}-1,9-dihydro-6H- | | |
| | purin-6-one | | |
| 152 | 1-(4-chlorophenyl)-9-methyl-2-[4,4,4- | 403, 405 | Example 2 |
| | trifluorobutylthio]-1,9-dihydro-6H-purin-6- | | |
| | one | | |
| 153 | 1-(4-chlorophenyl)-2-(2-[2-fluoro-4- | 466, 468 | Example |
| | trifluoromethylphenyl]ethylamino)-9- | | 53 |
| | methyl-1,9-dihydro-6H-purin-6-one | | |
| 154 | 1-(4-chlorophenyl)-2- | 389, 391 | Example 2 |
| | [cyclopentylmethylthio]-9-ethyl-1,9- | | |
| | dihydro-6H-purin-6-one | | |
| 155 | 1-(4-chlorophenyl)-9-methyl-2-[4,4,4- | 386, 388 | Example |
| | trifluorobutylamino]-1,9-dihydro-6H-purin- | | 53 |
| | 6-one | | |
| 156 | 3-(4-chlorophenyl)-2-[3,3,3- | 386, 388 | Example 2 |
| | trifluoropropylthio]pyrido[3,2-d]pyrimidin- | | |
| | 4(3H)-one | | |
| 157 | 4-{9-methyl-6-oxo-2-[4,4,4- | 394 | Example 2 |
| | trifluorobutylthio]-6,9-dihydro-1H-purin-1- | | |
| | yl}benzonitrile | | |
| 158 | 4-{9-ethyl-6-oxo-2-[3,3,3- | 394 | Example 2 |
| | trifluoropropylthio]-6,9-dihydro-1H-purin- | | |
| | 1-yl}benzonitrile | | |
| 159 | 1-(3-chloro-4-fluorophenyl)-9-methyl-2- | 421, 423 | Example 2 |
| | [4,4,4-trifluorobutylthio]-1,9-dihydro-6H- | | |
| | purin-6-one | | |
| 160 | 1-(3-chloro-4-fluorophenyl)-9-methyl-2- | 407, 409 | Example 2 |
| | [3,3,3-trifluoropropylthio]-1,9-dihydro-6H- | | |
| | purin-6-one | | |
| 161 | 1-(4-chlorophenyl)-9-methyl-2-{2-methyl- | 404, 406 | Example 2 |
| | 1,3-thiazol-4-ylmethylthio}-1,9-dihydro-6H- | | |
| | purin-6-one | | |
| 162 | 1-(4-chlorophenyl)-9-propyl-2-[3,3,3- | 417, 419 | Example 2 |
| | trifluoropropylthio]-1,9-dihydro-6H-purin- | | |
| | 6-one | | |
| 163 | 1-(4-chlorophenyl)-9-propyl-2-[4,4,4- | 431, 433 | Example 2 |
| | trifluorobutylthio]-1,9-dihydro-6H-purin-6- | | |
| | one | | |
| 164 | 1-(4-chlorophenyl)-9-isopropyl-2-[3,3,3- | 417, 419 | Example 2 |
| | trifluoropropylthio]-1,9-dihydro-6H-purin- | | |
| | 6-one | | |
| 165 | 1-(4-chlorophenyl)-9-isopropyl-2-[4,4,4- | 431, 433 | Example 2 |
| | trifluorobutylthio]-1,9-dihydro-6H-purin-6- | | |
| | one | | |
| 166 | 1-(4-chlorophenyl)-2-[3-fluoropropylthio]-9- | 353, 355 | Example 2 |
| | methyl-1,9-dihydro-6H-purin-6-one | | |
| 167 | 1-(4-chlorophenyl)-9-ethyl-2-[3- | 367, 369 | Example 2 |
| | fluoropropylthio]-1,9-dihydro-6H-purin-6- | | |
| | one | | |
| 168 | 1-(4-chlorophenyl)-9-methyl-2-(4- | 458, 460 | Example 2 |
| | trifluoromethyl-1,3-thiazol-2-ylmethylthio)- | | |
| | 1,9-dihydro-6H-purin-6-one | | |
| 169 | 1-(4-chlorophenyl)-9-methyl-2-(6- | 452, 454 | Example 2 |
| | trifluoromethylpyridin-3-ylmethylthio)-1,9- | | |
| | dihydro-6H-purin-6-one | | |
| 170 | 1-(4-chlorophenyl)-9-ethyl-2-(6- | 466, 468 | Example 2 |
| | trifluoromethylpyridin-3-ylmethylthio)-1,9- | | |
| | dihydro-6H-purin-6-one | | |
| 171 | 1-(4-chlorophenyl)-9-ethyl-2-(4- | 472, 474 | Example 2 |
| | trifluoromethyl-1,3-thiazol-2-ylmethylthio)- | | |
| | 1,9-dihydro-6H-purin-6-one | | |
| 172 | 4-[9-ethyl-6-oxo-2-(4-trifluoromethyl-1,3- | 463 | Example 2 |
| | thiazol-2-ylmethylthio)-6,9-dihydro-1H- | | |
| | purin-1-yl]benzonitrile | | |
| 173 | 1-(3,4-difluorophenyl)-9-methyl-2-[3,3,3- | 391 | Example 2 |
| | trifluoropropylthio]-1,9-dihydro-6H-purin- | | |
| | 6-one | | |
| 174 | 1-(3,4-difluorophenyl)-9-methyl-2-[4,4,4- | 405 | Example 2 |
| | trifluorobutylthio]-1,9-dihydro-6H-purin-6- | | |
| | one | | |
| 175 | 1-(4-chlorophenyl)-9-methyl-2-(2- | 458, 459, | Example 2 |
| | trifluoromethyl-1,3-thiazol-4-ylmethylthio)- | 460 | |
| | 1,9-dihydro-6H-purin-6-one | | |
| 176 | 1-(3-fluoro-4-methylphenyl)-9-methyl-2- | 387 | Example 2 |
| | [3,3,3-trifluoropropylthio]-1,9-dihydro-6H- | | |
| | purin-6-one | | |
| 177 | 1-(4-chlorophenyl)-9-methyl-2-(4- | 359, 361 | Example |
| | methylpiperazin-1-yl)-1,9-dihydro-6H- | | 53 |
| | purin-6-one | | |
| 178 | 1-(4-chloro-2-fluorophenyl)-9-methyl-2- | 407, 409 | Example 2 |
| | [3,3,3-trifluoropropylthio]-1,9-dihydro-6H- | | |
| | purin-6-one | | |
| 179 | 1-(4-fluorophenyl)-9-methyl-2-[3,3,3- | 373 | Example 2 |
| | trifluoropropylthio]-1,9-dihydro-6H-purin- | | |
| | 6-one | | |
| 180 | 1-(4-chloro-3-fluorophenyl)-9-methyl-2- | 407, 409 | Example 2 |
| | [3,3,3-trifluoropropylthio]-1,9-dihydro-6H- | | |
| | purin-6-one | | |
| 181 | 1-(4-chloro-3-methoxyphenyl)-9-methyl-2- | 419, 421 | Example 2 |
| | [3,3,3-trifluoropropylthio]-1,9-dihydro-6H- | | |
| | purin-6-one | | |
| 182 | 2-{1-(4-chlorophenyl)-9-ethyl-6-oxo-6,9- | 378, 380 | Example 2 |
| | dihydro-1H-purin-2-ylthio}-N- | | |
| | methylacetamide | | |
| 183 | 2-{1-(4-chlorophenyl)-9-ethyl-6-oxo-6,9- | 420, 422 | Example 2 |
| | dihydro-1H-purin-2-ylthio}-N,N- | | |
| | diethylacetamide | | |
| 184 | 1-(4-chlorophenyl)-9-ethyl-2-{5- | 402, 404 | Example 2 |
| | methylisoxazol-3-ylmethylthio}-1,9- | | |
| | dihydro-6H-purin-6-one | | |
| 185 | 1-(4-chlorophenyl)-9-methyl-2-methylthio- | 307, 309 | Example 2 |
| | 1,9-dihydro-6H-purin-6-one | | |
| 186 | 1-(4-fluorophenyl)-9-methyl-2-(4- | 442 | Example 2 |
| | trifluoromethyl-1,3-thiazol-2-ylmethylthio)- | | |
| | 1,9-dihydro-6H-purin-6-one | | |
| 187 | 9-ethyl-1-(4-fluorophenyl)-2-(4- | 456 | Example 2 |
| | trifluoromethyl-1,3-thiazol-2-ylmethylthio)- | | |
| | 1,9-dihydro-6H-purin-6-one | | |
| 188 | 1-(3-bromo-4-chlorophenyl)-9-methyl-2- | 467, 469, | Example 2 |
| | [3,3,3-trifluoropropylthio]-1,9-dihydro-6H- | 471 | |
| | purin-6-one | | |
| 189 | 1-(4-chlorophenyl)-2-cyclohexylamino-9- | 372, 374 | Example |
| | ethyl-1,9-dihydro-6H-purin-6-one | | 53 |
| 190 | 1-(4-chlorophenyl)-2-[3,3,3- | 375, 377 | Example 2 |
| | trifluoropropylthio]-1,9-dihydro-6H-purin- | | |
| | 6-one | | |
| 191 | 1-(4-chlorophenyl)-9-ethyl-2-{2-methyl-1,3- | 401, 403 | Example |
| | thiazol-4-ylmethylamino}-1,9-dihydro-6H- | | 53 |
| | purin-6-one | | |
| 192 | 3-(4-chlorophenyl)-2-(2-trifluoromethyl-1,3- | 455, 457 | Example 2 |
| | thiazol-4-ylmethylthio)pyrido[3,2- | | |
| | d]pyrimidin-4(3H)-one | | |
| 193 | 3-(4-chlorophenyl)-2-(2-trifluoromethyl-1,3- | 438, 440 | Example |
| | thiazol-4-ylmethylamino)pyrido[3,2- | | 50 |
| | d]pyrimidin-4(3H)-one | | |
| 194 | 1-(4-chloro-3-ethoxyphenyl)-9-ethyl-2- | 447, 449 | Example 2 |
| | [3,3,3-trifluoropropylthio]-1,9-dihydro-6H- | | |
| | purin-6-one | | |
| 195 | 1-(4-chloro-3-isopropoxyphenyl)-9-ethyl-2- | 461, 463 | Example 2 |
| | [3,3,3-trifluoropropylthio]-1,9-dihydro-6H- | | |
| | purin-6-one | | |
| 196 | 1-(4-chlorophenyl)-9-ethyl-2-{4-[3- | 504, 506 | Example |
| | trifluoromethylpyridin-2-yl]piperazin-1-yl}- | | 53 |
| | 1,9-dihydro-6H-purin-6-one | | |
| 197 | 3-(4-fluorophenyl)-2-(2-trifluoromethyl-1,3- | 439 | Example 2 |
| | thiazol-4-ylmethylthio)pyrido[3,2- | | |
| | d]pyrimidin-4(3H)-one | | |
| 198 | 6-(4-chlorophenyl)-5-(2-trifluoromethyl-1,3- | 461, 463 | Example 2 |
| | thiazol-4-ylmethylthio)[1,3]thiazolo[5,4- | | |
| | d]pyrimidin-7(6H)-one | | |
| 199 | 3-(4-chlorophenyl)-2- | 355, 357 | Example |
| | cyclohexylaminopyrido[3,2-d]pyrimidin- | | 50 |
| | 4(3H)-one | | |
| 200 | 3-(4-chlorophenyl)-2-[3,3,3- | 391, 393 | Example 2 |
| | trifluoropropylthio]thieno[3,2-d]pyrimidin- | | |
| | 4(3H)-one | | |
| 201 | 1-(4-chlorophenyl)-9-ethyl-2-(2- | 455, 457 | Example |
| | trifluoromethyl-1,3-thiazol-4- | | 53 |
| | ylmethylamino)-1,9-dihydro-6H-purin-6- | | |
| | one | | |
| 202 | 9-ethyl-1-(4-fluorophenyl)-2-(2- | 456 | Example 2 |
| | trifluoromethyl-1,3-thiazol-4-ylmethylthio)- | | |
| | 1,9-dihydro-6H-purin-6-one | | |
| 203 | 3-(4-chlorophenyl)-7-methyl-2-[3,3,3- | 405, 407 | Example 2 |
| | trifluoropropylthio]thieno[3,2-d]pyrimidin- | | |
| | 4(3H)-one | | |
| 204 | 3-(4-chlorophenyl)-7-methyl-2-(2-[4- | 464, 466 | Example |
| | trifluoromethylphenyl]ethylamino)thieno[3, | | 52 |
| | 2-d]pyrimidin-4(3H)-one | | |
| 205 | 3-(4-chlorophenyl)-7-methyl-2-[3,3,3- | 388, 390 | Example |
| | trifluoropropylamino]thieno[3,2- | | 52 |
| | d]pyrimidin-4(3H)-one | | |

### EXAMPLE 206

### 1-(4-Chlorophenyl)-2-{2-(2,4-dichlorophenyl)ethylthio}-9-[2-dimethulaminoethyl]-1,9-dihydro-6H-purin-6-one

A suspension of Example 143 (200 mg, 0.40 mmol) in CH₂Cl₂ (4 ml) was added to a suspension of triphenylphosphine (127 mg, 0.49 mmol), imidazole (33 mg, 0.49 mmol) and iodine (123 mg, 0.49 mmol) in CH₂C₂ (2 ml). The resulting yellow suspension was stirred at room temperature for 5 h. The reaction was diluted with CH₂Cl₂ (10 ml) and shaken with a 1:1 mixture of water and saturated aodium thiosulphate solution (15 ml). The aqueous layer was extracted with CH₂Cl₂ (2×20 ml) and the organic layers combined, dried over MgSO₄ and condensed to give the crude iodide (0.38 g). A portion of this iodide (125 mg) was taken up in a solution of dimethylamine (5.6 M in EtOH, 0.37 ml) and stirred at room temperature. After 3 h additional dimethylamine (5.6 M in EtOH, 0.3 ml) was added. After a further 90 min the reaction was condensed and taken up in fresh dimethylamine (5.6 M in EtOH, 2.0 ml) and the reaction heated at 50° C. in a sealed tube for 2 h. The reaction was condensed then loaded onto a strong cation exchange cartridge in MeOH and eluted using methanolic ammonia (2 M). The crude product was purified by preparative TLC, eluting with 7.5% methanol in CH₂Cl₂ to give the title compound (51 mg). ¹H NMR (360 MHz, CDCl₃) δ 7.80 (1H, s), 7.50 (2H, d, J8.7), 7.38 (1H, d, J2.2), 7.22-7.14 (4H, m), 4.23 (2H, t, J 6.3), 3.35 (2H, t, J7.4), 3.11 (2H, t, J7.6), 2.73 (2H, t, J6.4), 2.30 (6H, s); M/z (ES⁺) 526, 524, 525 (M+H⁺).

### EXAMPLE 207

### 1-(4-Chlorophenyl)-9-cyclopropyl-2-(2-[2-methoxy-4-trifluoromethylphenyl] ethylthio)-1,9-dihydro-6H-purin-6-one

To solution of NaH (60% in oil, 7 mg, 0.144 mmol) in DMF (0.8 ml) was added Example 111 (60 mg, 0.119 mmol) and the reaction stirred at room temperature for 15 min. Methyl iodide (10 μl, 0.155 mmol) was added and the reaction stirred for 3 h. The reaction was loaded onto a strong cation exchange cartridge with methanol and then the product eluted with methanolic ammonia (2 M). The crude product was purified by preparative TLC, eluting with 5% methanol in CH₂Cl₂ to give the title compound (52 mg, 84%). ¹H NMR (500 MHz, DMSO) δ 8.04 (1H, s), 7.62 (2H, d, J8.6), 7.42 (3H, m), 7.24 (2H, m), 3.84 (3H, s), 3.57-3.53 (1H, m), 3.36 (2H, t, J7.4), 3.05 (2H, t, J7.3), 1.15-1.11 (2H, m), 1.10-1.04 (2H, m). M/z (ES⁺) 521, 523 (M+H⁺).

### EXAMPLE 208

### 1-(4-Chlorophenyl)-9-ethyl-2-[methyl(3,3,3-trifluoropropyl)amino]-1,9-dihydro-6H-purin-6-one

Sodium hydride (60% in mineral oil, 10 mg, 0.26 mmol) was added to a solution of Example 141 (mono HCl salt, 45 mg, 0.12 mmol) in N,N-dimethylformamide (anhydrous, 2 ml), at RT and stirred for 5 min, effervescence seen. Methyl iodide (10 μl, 0.14 mmol) was added and the solution stirred for a further 20 min at room temperature. Water (2 ml) was added and the product extracted into ethyl acetate, which was dried over MgSO₄, and evaporated in vacuo to give an oil. The oil was dissolved in dichloromethane and loaded onto a strong cation exchange (SCX) cartridge. The cartridge was washed with dichloromethane and methanol then the material eluted with 2 M ammonia in methanol. This was concentrated in vacuo and purified by preparative thin layer chromatography (eluant: 2.5% methanol in dichloromethane with 0.1% saturated aqueous ammonia) to give the title compound as a glass (19 mg, 40%). ¹H NMR (400 MHz, CDCl₃) 67 7.62 (1H, s), 7.45 (2H, d, J8.7), 7.23 (2H, d, J8.6), 4.13 (2H, q, J7.3), 3.49-3.41 (2H, m), 2.53 (3H, s), 2.33-2.21 (2H, m), 1.52 (3H, t, J7.3). M/z (ES⁺) 400, 402 (M+H⁺).

### EXAMPLE 209

### 2-[[5-Chloro-1-benzothien-3-ylmethyl](methyl)amino]-1-(4-chlorophenyl)-9-ethyl-1,9-dihydro-6 H-purin-6-one

Prepared from Example 133 following the procedure described for Example 208. The title compound was purified by preparative thin layer chromatography (eluant: 5% methanol in dichloromethane with 0.1% saturated aqueous ammonia) to give the title compound as a solid (2 mg, 6%). ¹H NMR (500 MHz, DMSO) 67 8.00 (1H, d, J8.5), 7.97 (1H, s), 7.84 (1H, s), 7.62 (1H, s), 7.43-7.37 (5H m), 4.54 (2H, s), 4.12 (2H, q, J7.3), 2.42 (3H, s), 1.39 (3H, t, J7.2). M/z (ES⁺) 484, 485, 486 (M+H⁺).

### EXAMPLE 210

### 2-Chloro-5-{9-methyl-6-oxo-2-[3,3,3-trifluoropropylthio]-6,9-dihydro-1H-purin-1-yl}benzonitrile

A mixture of Example 188 (114 mg, 0.243 mmol), zinc cyanide (17 mg, 0.146 mmol), zinc dust (nanosize activated powder, 2 mg) and [1,1′-bis(diphenylphosphino)ferrocene]dichloropalladium (II) (5 mg) in N,N-dimethylacetamide (3 ml) was heated in a microwave reactor at 160° C. for 20 minutes. The mixture was filtered through Celite®, washing through with water (25 ml) and ethyl acetate (25 ml). The layers were separated and the aqueous phase extracted with more ethyl acetate (25 ml). The combined organic layers were washed with water (3×25 ml), brine (25 ml), dried over sodium sulphate and evaporated. Purification by mass-directed preparative HPLC gave the title compound (31 mg, 31%). ¹H NMR (400 MHz, DMSO) δ 8.25 (1H, d, J2.4), 8.07 (1H, s), 8.01 (1H, d, J8.6), 7.88 (1H, dd, J2.4, 8.6), 3.76 (3H, s), 3.37-3.27 (2H, m), 2.79-2.67 (2H, m); M/z (ES⁺) 414, 416 (M+H⁺).

The above exemplified compounds of the present invention have been tested in the following assay and generally possess an IC₅₀<300 nM and, in the majority of cases, <200 nM. Other assays, such as electrophysiology using rat VR1 expressed in HEK cells measuring activity at various pH levels, can be used.

Biological Methodology

Determination of in vitro activity CHO cells, stably expressing recombinant human VR1 receptors and plated into black-sided 384-well plates, were washed twice with assay buffer (Hepes-buffered saline) and then incubated with 1 uM Fluo-3-AM for 60 minutes in darkness. Cells were washed twice more to remove excess dye, before being placed, along with plates containing capsaicin and test compounds in a Molecular Devices FLIPR. The FLIPR simultaneously performed automated pharmacological additions and recorded fluorescence emission from Fluo-3. In all experiments, basal fluorescence was recorded, before addition of test compounds and subsequent addition of a previously determined concentration of capsaicin that evoked 80% of the maximum response. Inhibition of capsaicin evoked increases in intracellular [Ca²⁺] were expressed relative to wells on the same plate to which capsaicin was added in the absence of test compounds. Increases in intracellular [Ca²⁺] occuring after addition of test compound alone, prior to addition of capsaicin, allow determination of intrinsic agonist or partial agonist activity, if present.

1. A compound of formula (I): 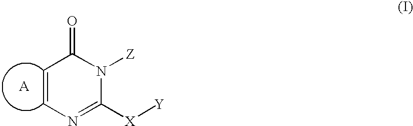
wherein:
A is a benzene ring, a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;
A is optionally substituted by one, two or three groups independently chosen from halogen, hydroxy, S(O)ᵣC₁₋₄alkyl, S(O)ᵣNR⁵R⁶, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl; and a phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁵R⁶ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;
X is O, S or NR¹ where R¹ is hydrogen or C₁₋₆alkyl;
Y is (CR²R³)ₙ(CO)ₚ(NR⁴)_{q}W;
R² and R³ are independently hydrogen, hydroxy, halogen or C₁₋₄alkyl;
R⁴ is hydrogen or C₁₋₆alkyl;
n is zero, one, two, three or four;
p is zero or one;
q is zero or one;
r is zero, one or two;
W is hydrogen, C₁₋₆alkoxy, haloC₁₋₆alkoxy, C₁₋₆alkyl, haloC₁₋₆alkyl, hydroxyC₁₋₆alkyl, aminoC₁₋₆alkyl, carboxyC₁₋₆alkyl, C₃₋₇cycloalkyl, haloC₃₋₇cycloalkyl; or a phenyl ring, a five-membered heteroaromatic ring containing one, two, three or four heteroatoms independently chosen from O, N and S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, a six-membered saturated ring containing one or two heteroatoms independently chosen from O and N, the ring being optionally substituted by halogen, C₁₋₆alkyl, C₂₋₆alkenyl, C₂₋₆alkynyl, nitro, cyano, C₃₋₇cycloalkyl, hydroxy, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described or NR⁵R⁶;
each R⁵ and R⁶ is independently hydrogen or C₁₋₆alkyl or R⁵ and R⁶, together with the nitrogen atom to which they are attached, may form a saturated 4-7 membered ring;
Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by halogen, hydroxy, cyano, nitro, NR⁵R⁶ as defined above, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;
when R¹ and R⁴ are alkyl groups they may, together with the nitrogen atoms to which they are attached, form a piperazine ring;
or a pharmaceutically acceptable salt or tautomer thereof.

2. A compound of claim 1 represented by formula (I)¹: 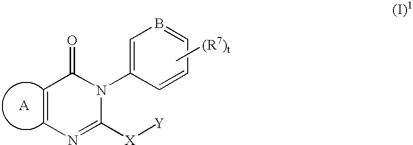
wherein:
B is N or CH;
t is 1, 2 or 3;
A is a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;
A is optionally substituted by halogen, C₁₋₄alkyl, hydroxyC₁₋₄alkyl; C₃₋₇cycloalkyl, C₃₋₇cycloalkylC₁₋₄alkyl, phenyl or di(C₁₋₄alkyl)aminoC₁₋₄alkyl;
X is O, S or NR¹ where R¹ is hydrogen or C₁₋₄alkyl;
Y is (CR²R³)ₙ(CO)ₚ(NR⁴)_{q}W, where R², R³, R⁴, n, p and q are as defined in claim 1;
W is hydrogen, C₁₋₆alkoxy, C₁₋₆alkyl, haloC₁₋₆alkyl, C₃₋₇cycloalkyl; or a phenyl ring, a five-membered heteroaromatic ring containing one, two, three or four heteroatoms independently chosen from O, N or S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, the ring being optionally substituted by halogen, C₁₋₄alkyl, hydroxy, C₁₋₄alkoxy, haloC₁₋₄alkyl, phenyl, haloC₁₋₄alkoxy or NR⁵R⁶ where R⁵ and R⁶ are independently C₁₋₄alkyl or, R⁵ and R⁶, together with the nitrogen atom to which they are attached, form a 5-6 membered saturated ring;
R⁷ is hydrogen, cyano, halogen, C₁₋₄alkyl, haloC₁₋₄alkyl, C₁₋₄alkoxy, haloC₁₋₄alkoxy, amino, C₁₋₄alkylamino or di(C₁₋₄alkyl)amino;
when R¹ and R⁴ are alkyl groups they may, together with the nitrogen atoms to which they are attached, form a piperazine ring; or a pharmaceutically acceptable salt or tautomer thereof.

3. A compound of claim 1 or 2 represented by formula (IA): 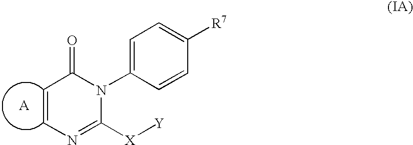
wherein A is a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;
A is optionally substituted by halogen, C₁₋₄alkyl, C₃₋₇cycloalkyl or phenyl;
X is O, S or NR¹ where R¹ is hydrogen or C₁₋₄alkyl;
Y is (CR²R³)ₙ(CO)ₚ(NR⁴)_{q}W, where R², R³, R⁴, n, p and q are as defined for formula I;
W is hydrogen, C₁₋₆alkyl, haloC₁₋₆alkyl, C₃₋₇cycloalkyl; or a phenyl ring, a five-membered heteroaromatic ring containing one, two, three or four heteroatoms independently chosen from O, N or S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, the ring being optionally substituted by halogen, C₁₋₄alkyl, hydroxy, C₁₋₄alkoxy, haloC₁₋₄alkyl, phenyl, haloC₁₋₄alkoxy or NR⁵R⁶ where R⁵ and R⁶ are independently C₁₋₄alkyl or, R⁵ and R⁶, together with the nitrogen atom to which they are attached, form a 5-6 membered saturated ring;
R⁷ is hydrogen, cyano, halogen, C₁₋₄alkyl, haloC₁₋₄alkyl, C₁₋₄alkoxy or haloC₁₋₄alkoxy;
when R¹ and R⁴ are alkyl groups they may, together with the nitrogen atoms to which they are attached, form a piperazine ring;
or a pharmaceutically acceptable salt or tautomer thereof.

4. A compound selected from:
3-(4-chlorophenyl)-2-[3-fluorobenzylthio]pyrido[3,4-d]pyrimidin-4(3H)-one;
3-(4-chlorophenyl)-2-{2-(4-chlorophenyl)-2-oxoethylthio}pyrido[3,2-d]pyrimidin-4(3H)-one;
3-(4-chlorophenyl)-2-[3-fluorobenzylthio]pyrido[3,2-d]pyrimidin-4(3H)-one;
3-(4-chlorophenyl)-2-{2-(4-chlorophenyl)ethylthio}pyrido[3,2-d]pyrimidin-4(3H)-one;
2-{5-chloro-1-benzothien-3-ylmethylthio}-3-(4-chlorophenyl)pyrido[3,2-d]pyrimidin-4(3H)-one;
2-[1-benzothien-3-ylmethyl)thio]-3-(4-chlorophenyl)pyrido[3,2-d]pyrimidin-4(3H)-one;
2-[1,3-benzothiazol-2-ylmethylthio]-3-(4-chlorophenyl)pyrido[3,2-d]pyrimidin-4(3H)-one;
3-(4-chlorophenyl)-2-[2-oxo-2-phenylethylthio]pyrido[3,2-d]pyrimidin-4(3H)-one;
3-(4-chlorophenyl)-2-{2-(3-chlorophenyl)-2-oxoethylthio}pyrido[3,2-d]pyrimidin-4(3H)-one;
3-(4-chlorophenyl)-2-(2-oxo-2-[4-trifluoromethoxyphenyl]ethylthio)pyrido[3,2-d]pyrimidin-4(3H)-one;
3-(4-chlorophenyl)-2-(2-oxo-2-[4-trifluoromethylphenyl]ethylthio)pyrido[3,2-d]pyrimidin-4(3H)-one;
3-(4-chlorophenyl)-2-{2-oxo-2-(4-pyrrolidin-1-ylphenyl)ethylthio}pyrido[3,2-d]pyrimidin-4(3H)-one;
3-(4-chlorophenyl)-2-[2-oxo-2-pyridin-2-ylethylthio]pyrido[3,2-d]pyrimidin-4(3H)-one;
3-(4-chlorophenyl)-2-[4-fluorobenzylthio]pyrido[3,2-d]pyrimidin-4(3H)-one;
2-[3-chlorobenzylthio]-3-(4-chlorophenyl)pyrido[3,2-d]pyrimidin-4(3H)-one;
3-(4-chlorophenyl)-2-[pyridin-2-ylmethylthio]pyrido[3,2-d]pyrimidin-4(3H)-one;
3-(4-chlorophenyl)-2-{5-phenyl-1,2,4-oxadiazol-3-ylmethylthio}pyrido[3,2-d]pyrimidin-4(3H)-one;
2-{3-(4-chlorophenyl)-4-oxo-3,4-dihydropyrido[3,2-d]pyrimidin-2-ylthio}-N-(5-methylisoxazol-3-yl)acetamide;
3-(4-chlorophenyl)-2-[3-fluorobenzylthio]thieno[2,3-d]pyrimidin-4(3H)-one;
3-(4-chlorophenyl)-2-[3-fluorobenzylthio]thieno[3,2-d]pyrimidin-4(3H)-one;
3-(4-chlorophenyl)-2-{2-(4-chlorophenyl)-2-oxoethylthio}thieno [3,2-d]pyrimidin-4(3H)-one;
6-(4-chlorophenyl)-5-[3-fluorobenzylthio][1,3]thiazol[5,4-d]pyrimidin-7(6H)-one;
6-(4-chlorophenyl)-5-{2-(4-chlorophenyl)-2-oxoethylthio}[1,3]thiazol[5,4-d]pyrimidin-7(6H)-one;
6-(4-chlorophenyl)-5-{2-(4-chlorophenyl)-2-oxoethylthio}[1,3]thiazol[4,5-d]pyrimidin-7(6H)-one;
2-{5-chloro-1-benzothien-3-ylmethylthio}-1-(4-chlorophenyl)-9-methyl-1,9-dihydro-6H-purin-6-one;
1-(4-chlorophenyl)-9-methyl-2-(2-[4-trifluoromethylphenyl]ethylthio)-1,9-dihydro-6H-purin-6-one;
1-(4-chlorophenyl)-2-{2-(4-chlorophenyl)ethylthio}-9-methyl-1,9-dihydro-6H-purin-6-one;
1-(4-chlorophenyl)-2-{2-(4-chlorophenyl)-2-oxoethylthio}-9-methyl-1,9-dihydro-6H-purin-6-one;
1-(4-chlorophenyl)-2-[3-fluorobenzylthio]-9-methyl-1,9-dihydro-6H-purin-6-one;
1-(4-chlorophenyl)-2-[3-fluorobenzylthio]-1,9-dihydro-6H-purin-6-one;
2-{2-(4-chlorophenyl)-2-oxoethylthio}-3-[4-trifluoromethylphenyl]pyrido[3,2-d]pyrimidin-4(3H)-one;
2-[3-fluorobenzylthio]-3-[4-trifluoromethylphenyl]pyrido[3,2-d]pyrimidin-4(3H)-one;
2-(methylthio)-3-pyridin-3-ylpyrido[3,2-d]pyrimidin-4(3H)-one;
3-(4-chlorophenyl)-2-(3-oxo-4-phenylpiperazin-1-yl)pyrido[3,2-d]pyrimidin-4(3H)-one;
3-4-chlorophenyl-2-{2-(4-chloropheny1)ethylamino}pyrido[3,2-d]pyrimidin-4(3H)-one;
3-(4-chlorophenyl)-2-[3-fluorobenzyloxy]thieno[3,2-d]pyrimidin-4(3H)-one;
3-(4-chlorophenyl)-2-[3-fluorobenzylamino]thieno[3,2-d]pyrimidin-4(3H)-one;
1-(4-chlorophenyl)-2-[2-cyclohexylethylthio]-9-methyl-1,9-dihydro-6H-purin-6-one;
1-(4-chlorophenyl)-2-[2-cyclohexylethylthio]-9-ethyl-1,9-dihydro-6H-purin-6-one;
1-(4-chlorophenyl)-9-ethyl-2-[3,3,3-trifluoropropylthio]-1,9-dihydro-6H-purin-6-one;
1-(4-chlorophenyl)-9-ethyl-2-propylthio-1,9-dihydro-6H-purin-6-one;
1-(4-chlorophenyl)-2-[cyclopropylmethylthio]-9-ethyl-1,9-dihydro-6H-purin-6-one;
1-(4-chlorophenyl)-9-methyl-2-[3,3,3-trifluoropropylthio]-1,9-dihydro-6H-purin-6-one;
1-(4-chlorophenyl)-9-cyclopropyl-2-[3,3,3-trifluoropropylthio]-1,9-dihydro-6H-purin-6-one;
1-(4-chlorophenyl)-9-ethyl-2-[2,2,2-trifluoroethylthio]-1,9-dihydro-6H-purin-6-one;
1-(4-chlorophenyl)-9-ethyl-2-[4,4,4-trifluorobutylthio]-1,9-dihydro-6H-purin-6-one;
1-(4-chlorophenyl)-9-phenyl-2-(2-[4-trifluoromethylphenyl]ethylthio)-1,9-dihydro-6H-purin-6-one;
1-(4-chlorophenyl)-2-methylthio-9-phenyl-1,9-dihydro-6H-purin-6-one;
1-(4-chlorophenyl)-9-phenyl-2-[3,3,3-trifluoropropylthio]-1,9-dihydro-6H-purin-6-one;
1-(4-chlorophenyl)-9-phenyl-2-[4,4,4-trifluorobutylthio]-1,9-dihydro-6H-purin-6-one;
4-{9-methyl-6-oxo-2-[3,3,3-trifluoropropylthio]-6,9-dihydro-1H-purin-1-yl}benzonitrile;
9-methyl-1-(4-methylphenyl)-2-[3,3,3-trifluoropropylthio]-1,9-dihydro-6H-purin-6-one;
1-(4-chlorophenyl)-9-ethyl-2-(2-[4-trifluoromethylphenyl]ethylamino)-1,9-dihydro-6H-purin-6-one;
1-(4-chlorophenyl)-9-ethyl-2-(2-[2-fluoro-4-trifluoromethylphenyl]ethylamino)-1,9-dihydro-6H-purin-6-one;
3-(4-chlorophenyl)-2-methylaminothieno[3,2-d]pyrimidin-4(3H)-one;
3-(4-chlorophenyl)-2-{2-(2-chlorophenyl)-2-oxoethylthio}pyrido[3,2-d]pyrimidin-4(3H)-one;
2-{2-(4-3-(chlorophenyl)-2-oxoethylthio}-3-phenylpyrido[3,2-d]pyrimidin-4(3H)-one;
3-(4-chlorophenyl)-2-[2-phenylethylthio]pyrido[3,2-d]pyrimidin-4(3H)-one;
3-(4-chlorophenyl)-2-[2-fluorobenzylthio]pyrido[3,2-d]pyrimidin-4(3H)-one;
6-(4-chlorophenyl)-5-{2-(4-chlorophenyl)ethylthio}[1,3]thiazol[5,4-d]pyrimidin-7(6H)-one;
3-(4-chlorophenyl)-2-{2-(3-chlorophenyl)ethylthio}pyrido[3,2-d]pyrimidin-4(3H)-one;
3-(4-chlorophenyl)-2-(2-[4-trifluoromethylphenyl]ethylthio)pyrido[3,2-d]pyrimidin-4(3H)-one;
6-(4-chlorophenyl)-5-[3-fluorobenzylthio][1,3]thiazol[4,5-d]pyrimidin-7(6H)-one;
6-(4-chlorophenyl)-5-{2-(4-chlorophenyl)ethylthio}[1,3]thiazol[4,5-d]pyrimidin-7(6H)-one;
2-{6-chloro-1-benzothien-3-ylmethylthio}-1-(4-chlorophenyl)-9-methyl-1,9-dihydro-6H-purin-6-one;
2-{5-chloro-1-benzothien-3-ylmethylthio}-1-(4-chlorophenyl)-9-ethyl-1,9-dihydro-6H-purin-6-one;
2-{5-chloro-1-benzothien-3-ylmethylthio}-1-(4-chlorophenyl)-9-isopropyl-1,9-dihydro-6H-purin-6-one;
3-(6-chloropyridin-3-yl)-2-[3-fluorobenzylthio]thieno[3,2-d]pyrimidin-4(3H)-one;
2-{5-chloro-1-benzothien-3-ylmethylthio}-3-[4-trifluoromethylphenyl]pyrido[3,2-d]pyrimidin-4(3H)-one;
1-(3-chlorophenyl)-9-methyl-2-(2-[4-trifluoromethylphenyl]ethylthio)-1,9-dihydro-6H-purin-6-one;
1-(4-chlorophenyl)-2-[3,4-dichlorobenzylthio]-9-methyl-1,9-dihydro-6H-purin-6-one;
1-(4-chlorophenyl)-9-cyclopropyl-2-(2-[4-trifluoromethylphenyl]ethylthio)-1,9-dihydro-6H-purin-6-one;
1-(4-chlorophenyl)-9-ethyl-2-(2-[4-trifluoromethylphenyl]ethylthio)-1,9-dihydro-6H-purin-6-one;
3-[4-trifluoromethylphenyl]-2-(2-[4-trifluoromethylphenyl]ethylthio)thieno[3,2-d]pyrimidin-4(3H)-one;
3-[4-trifluoromethylphenyl]-2-(2-[4-trifluoromethylphenyl]ethylthio)pyrido[3,2-d]pyrimidin-4(3H)-one;
1-(4-chlorophenyl)-2-{2-(2,4-dichlorophenyl)ethylthio}-9-methyl-1,9-dihydro-6H-purin-6-one;
3-(4-chlorophenyl)-2-[3,4-dichlorobenzylthio]pyrido[3,2-d]pyrimidin-4(3H)-one;
2-[3-chloro-4-fluorobenzylthio]-3-(4-chlorophenyl)pyrido[3,2-d]pyrimidin-4(3H)-one;
1-(4-chlorophenyl)-2-(2-[3-fluoro-4-trifluoromethylphenyl]ethylthio)-9-methyl-1,9-dihydro-6H-purin-6-one;
3-(4-fluorophenyl)-2-(2-[4-trifluoromethylphenyl]ethylthio)pyrido[3,2-d]pyrimidin-4(3H)-one;
1-(4-fluorophenyl)-9-methyl-2-(2-[4-trifluoromethylphenyl]ethylthio)-1,9-dihydro-6H-purin-6-one;
1-(4-chlorophenyl)-9-methyl-2-(2-[4-trifluoromethoxyphenyl]ethylthio)-1,9-dihydro-6H-purin-6-one;
1-(4-chlorophenyl)-2-{2-(4-fluorophenyl)ethylthio}-9-methyl-1,9-dihydro-6H-purin-6-one;
1-(4-chlorophenyl)-2-[2,4-dichlorobenzylthio]-9-methyl-1,9-dihydro-6H-purin-6-one;
2-{5-chloro-1-benzothien-3-ylmethylthio}-9-methyl-1-[4-trifluoromethylphenyl]-1,9-dihydro-6H-purin-6-one;
9-methyl-1-[4-trifluoromethylphenyl]-2-(2-[4-trifluoromethylphenyl]ethylthio)-1,9-dihydro-6H-purin-6-one;
4-[2-{5-chloro-1-benzothien-3-ylmethylthio}-4-oxopyrido[3,2-d]pyrimidin-3(4H)-yl]benzonitrile;
ethyl {1-(4-chlorophenyl)-9-methyl-6-oxo-6,9-dihydro-1H-purin-2-ylthio}acetate;
4-[4-oxo-2-(2-[4-trifluoromethylphenyl]ethylthio)pyrido[3,2-d]pyrimidin-3(4H)-yl]benzonitrile;
1-(4-chlorophenyl)-2-[3,4-dichlorobenzylthio]-9-ethyl-1,9-dihydro-6H-purin-6-one;
1-(4-chlorophenyl)-9-propyl-2-(2-[4-trifluoromethylphenyl]ethylthio)-1,9-dihydro-6H-purin-6-one;
1-(4-chlorophenyl)-9-methyl-2-(2-[4-trifluoromethylphenyl]ethylamino)-1,9-dihydro-6H-purin-6-one;
1-(4-chlorophenyl)-2-[3,4-dichlorobenzylthio]-9-propyl-1,9-dihydro-6H-purin-6-one;
2-{5-chloro-1-benzothien-3-ylmethylthio}-9-methyl-1-[4-trifluoromethoxyphenyl]-1,9-dihydro-6H-purin-6-one;
2-{5-chloro-1-benzothien-3-ylmethylthio}-1-(4-chlorophenyl)-9-(cyclopropylmethyl)-1,9-dihydro-6H-purin-6-one;
1-(4-chlorophenyl-2-{2-(4-chlorophenyl-2-oxoethylthio}-9-(cyclopropylmethyl)-1,9-dihydro-6H-purin-6-one;
1-(4-chlorophenyl)-9-cyclopropyl-2-[3-fluorobenzylthio]-1,9-dihydro-6H-purin-6-one;
2-[3-chloro-4-fluorobenzylthio]-1-(4-chlorophenyl)-9-cyclopropyl-1,9-dihydro-6H-purin-6-one;
1-(4-chlorophenyl)-9-cyclopropyl-2-[3,4-dichlorobenzylthio]-1,9-dihydro-6H-purin-6-one;
1-(4-chlorophenyl)-9-cyclopropyl-2-{3-trifluoromethylbenzylthio}-1,9-dihlydro-6H-purin-6-one;
2-[3-chlorobenzylthio]-1-(4-chlorophenyl)-9-cyclopropyl-1,9-dihydro-6H-purin-6-one;
2-{5-chloro-1-benzothien-3-ylmethylthio}-1-(4-chlorophenyl)-9-cyclopropyl-1,9-dihydro-6H-purin-6-one;
1-(4-chlorophenyl)-2-{2-(4-chlorophenyl)-2-oxoethylthio}-9-cyclopropyl-1,9-dihydro-6H-purin-6-one;
1-(4-chlorophenyl)-9-cyclopropyl-2-(2-oxo-2-[4-trifluoromethylphenyl]ethylthio)-1,9-dihydro-6H-purin-6-one;
1-(4-chlorophenyl)-9-cyclopropyl-2-{2-(4-fluorophenyD)-2-oxoethylthio}-1,9-dihydro-6H-purin-6-one;
1-(4-chlorophenyl)-9-cyclopropyl-2-[2,4-dichlorobenzylthio]-1,9-dihydro-6H-purin-6-one;
1-(4-chlorophenyl)-9-cyclopropyl-2-{2-(2,4-dichlorophenyl)ethylthio}-1,9-dihydro-6H-purin-6-one;
3-(4-chlorophenyl)-2-[3-fluorobenzylthio]-7-methylthieno[3,2-d]pyrimidin-4(3H)-one;
2-{5-chloro-1-benzothien-3-ylmethylthio}-3-(4-chlorophenyl) -7-methylthieno[3,2-d]pyrimidin-4(3H)-one;
4-[2-[3-fluorobenzylthio]-4-oxopyrido[3,2-d]pyrimidin-3(4H)-yl]benzonitrile;
1-(4-chlorophenyl)-9-cyclopropyl-2-(2-[2-hydroxy-4-trifluoromethylphenyl]ethylthio)-1,9-dihydro-6H-purin-6-one;
2-{5-chloro-1-benzothien-3-ylmethylthio}-9-ethyl-1-(4-methylphenyl)-1,9-dihydro-6H-purin-6-one;
2-{5-chloro-1-benzothien-3-ylmethylthio}-1-(4-chlorophenyl)-9-propyl-1,9-dihydro-6H-purin-6-one;
1-(4-chlorophenyl)-9-cyclopropyl-2-(2-[2-fluoro-4-trifluoromethylphenyl]ethylthio)-1,9-dihydro-6H-purin-6-one;
1-(4-bromophenyl)-2-{5-chloro-1-benzothien-3-ylmethylthio}-9-ethyl-1,9-dihydro-6H-purin-6-one;
2-[1,3-benzothiazol-2-ylmethylthio]-1-(4-chlorophenyl)-9-cyclopropyl-1,9-dihydro-6H-purin-6-one;
1-(4-chlorophenyl)-9-cyclopropyl-2-{2-fluoro-4-trifluoromethylbenzylthio}-1,9-dihydro-6H-purin-6-one;
1-(4-chlorophenyl)-9-cyclopropyl-2-{2-fluoro-5-trifluoromethylbenzylthio}-1,9-dihydro-6H-purin-6-one;
1-(4-chlorophenyl)-9-cyclopropyl-2-{3-fluoro-4-trifluoromethylbenzylthio}-1,9-dihydro-6H-purin-6-one;
1-(4-chlorophenyl)-9-cyclopropyl-2-(5-trifluoromethyl-1,3-benzothiazol-2-ylmethylthio)-1,9-dihydro-6H-purin-6-one;
2-{5-chloro-1-benzothien-3-ylmethylthio}-1-[4-dimethylaminophenyl]-9-ethyl-1,9-dihydro-6H-purin-6-one;
3-(4-chlorophenyl)-2-[3,4-dichlorobenzylthio]-7-methylthieno[3,2-d]pyrimidin-4(3H)-one;
2-{2-(2,4-dichlorophenyl)ethylthio}-1-(4-fluorophenyl-9-methyl-1,9-dihydro-6H-purin-6-one;
1-(4-chlorophenyl)-2-{2-(2,4-dichlorophenyl)ethylthio}-9-ethyl-1,9-dihydro-6H-purin-6-one;
1-(4-chlorophenyl)-9-ethyl-2-pentylthio-1,9-dihydro-6H-purin-6-one;
1-(4-chlorophenyl)-9-ethyl-2-[3-methylbutylthio]-1,9-dihydro-6H-purin-6-one;
1-(4-chlorophenyl)-2-[2-cyclohexylethylamino]-9-ethyl-1,9-dihydro-6H-purin-6-one;
1-(4-chlorophenyl)-2-(2-[2-chloro-4-trifluoromethylphenyl]ethylthio)-9-methyl-1,9-dihydro-6H-purin-6-one;
1-(4-chlorophenyl)-2-(2-[2-chloro-4-trifluoromethylphenyl]ethylthio)-9-ethyl-1,9-dihydro-6H-purin-6-one;
2-{2-(2,4-dichlorophenyl)ethylthio}-9-ethyl-1-(4-fluorophenyl)-1,9-dihydro-6H-purin-6-one;
9-ethyl-1-(4-fluorophenyl) -2-(2-[2-fluoro-4-trifluoromethylphenyl]ethylthio)-1,9-dihydro-6H-purin-6-one;
2-(2-[2-chloro-4-trifluoromethylphenyl]ethylthio)-9-ethyl-1-(4-fluorophenyl)-1,9-dihydro-6H-purin-6-one;
2-{5-chloro-1-benzothien-3-ylmethylamino}-1-(4-chlorophenyl)-9-ethyl-1,9-dihydro-6H-purin-6-one;
1-(4-chlorophenyl)-9-ethyl-2-[3-fluorobenzylamino]-1,9-dihydro-6H-purin-6-one;
2-{5-chloro-1,3-benzothiazol-2-ylmethylthio}-1-(4-chlorophenyl)-9-ethyl-1,9-dihydro-6H-purin-6-one;
1-(2,4-dichlorophenyl)-2-{2-(2,4-dichlorophenyl)ethylthio}-9-methyl-1,9-dihydro-6H-purin-6-one;
1-(4-chlorophenyl)-2-(2-[2-fluoro-4-trifluoromethylphenyflethylthio)-9-methyl-1,9-dihydro-6H-purin-6-one;
1-(4-chlorophenyl)-9-ethyl-2-(2-[2-fluoro-4-trifluoromethylphenyl]ethylthio)-1,9-dihydro-6H-purin-6-one;
1-(4-chlorophenyl)-2-{2-(2,4-dichlorophenyl)-2-oxoethylthio}-9-methyl-1,9-dihydro-6H-purin-6-one;
9-ethyl-1-(4-fluorophenyD)-2-[3,3,3-trifluoropropylthio]-1,9-dihydro-6H-purin-6-one;
1-(4-chlorophenyl)-9-ethyl-2-[3,3,3-trifluoropropylamino]-1,9-dihydro-6H-purin-6-one;
9-cyclopropyl-2-{2-(2,4-dichlorophenyl)ethylthio}-1-(4-fluorophenyl)-1,9-dihydro-6H-purin-6-one;
1-(4-chlorophenyl)-2-{2-(2,4-dichlorophenyl)ethylthio}-9-(2-hydroxyethyl)-1,9-dihydro-6H-purin-6-one;
9-cyclopropyl-1-(4-fluorophenyl)-2-(3,3,3-trifluoropropylthio]-1,9-dihydro-6H-purin-6-one;
1-(4-chlorophenyl)-2-{2-(2,4-dichlorophenyl)ethylamino}-9-ethyl-1,9-dihydro-6H-purin-6-one;
1-(4-chlorophenyl)-2-[cyclobutylmethylthio]-9-ethyl-1,9-dihydro-6H-purin-6-one;
9-cyclopropyl-1-(4-fluorophenyl)-2-(2-[2-fluoro-4-trifluoromethylphenyl]ethylthio)-1,9-dihydro-6H-purin-6-one;
1-(4-chlorophenyl)-9-ethyl-2-[3,3,3-trifluoro-2-hydroxypropylthio]-1,9-dihydro-6H-purin-6-one;
1-(4-chlorophenyl)-9-ethyl-2-[3,3,3-trifluoro-2,2-dihydroxypropylthio]-1,9-dihydro-6H-purin-6-one;
1-(4-chlorophenyl)-2-[2-cyclopentylethylthio]-9-ethyl-1,9-dihydro-6H-purin-6-one;
1-(4-chlorophenyl)-9-ethyl-2-{2-methyl-1,3-thiazol-4-ylmethylthio}-1,9-dihydro-6H-purin-6-one;
1-(4-chlorophenyl)-9-methyl-2-[4,4,4-trifluorobutylthio]-1,9-dihydro-6H-purin-6-one;
1-(4-chlorophenyl)-2-(2-[2-fluoro-4-trifluoromethylphenyl]ethylamino)-9-methyl-1,9-dihydro-6H-purin-6-one;
1-(4-chlorophenyl)-2-[cyclopentylmethylthio]-9-ethyl-1,9-dihydro-6H-purin-6-one;
1-(4-chlorophenyl)-9-methyl-2-[4,4,4-trifluorobutylamino]-1,9-dihydro-6H-purin-6-one;
3-(4-chlorophenyl)-2-[3,3,3-trifluoropropylthio]pyrido[3,2-d]pyrimidin-4(3H)-one;
4-{9-methyl-6-oxo-2-[4,4,4-trifluorobutylthio]-6,9-dihydro-1H-purin-1-yl}benzonitrile;
4-{9-ethyl-6-oxo-2-[3,3,3-trifluoropropylthio]-6,9-dihydro-1H-purin-1-yl}benzonitrile;
1-(3-chloro-4-fluorophenyl)-9-methyl-2-[4,4,4-trifluorobutylthio]-1,9-dihydro-6H-purin-6-one;
1-(3-chloro-4-fluorophenyl)-9-methyl-2-[3,3,3-trifluoropropylthio]-1,9-dihydro-6H-purin-6-one;
1-(4-chlorophenyl)-9-methyl-2-{2-methyl-1,3-thiazol-4-ylmethylthio}-1,9-dihydro-6H-purin-6-one;
1-(4-chlorophenyl)-9-propyl-2-[3,3,3-trifluoropropylthio]-1,9-dihydro-6H-purin-6-one;
1-(4-chlorophenyl)-9-propyl-2-[4,4,4-trifluorobutylthio)]-1,9-dihydro-6H-purin-6-one;
1-(4-chlorophenyl)-9-isopropyl-2-[3,3,3-trifluoropropylthio]-1,9-dihydro-6H-purin-6-one;
1-(4-chlorophenyl)-9-isopropyl-2-[4,4,4-trifluorobutylthio]-1,9-dihydro-6H-purin-6-one;
1-(4-chlorophenyl)-2-[3-fluoropropylthio]-9-methyl-1,9-dihydro-6H-purin-6-one;
1-(4-chlorophenyl)-9-ethyl-2-[3-fluoropropylthio]-1,9-dihydro-6H-purin-6-one;
1-(4-chlorophenyl)-9-methyl-2-(4-trifluoromethyl-1,3-thiazol-2-ylmethylthio)-1,9-dihydro-6H-purin-6-one;
1-(4-chlorophenyl)-9-methyl-2-(6-trifluoromethylpyridin-3-ylmethylthio)-1,9-dihydro-6H-purin-6-one;
1-(4-chlorophenyl)-9-ethyl-2-(6-trifluoromethylpyridin-3-ylmethylthio)-1,9-dihydro-6H-purin-6-one;
1-(4-chlorophenyl)-9-ethyl-2-(4-trifluoromethyl-1,3-thiazol-2-ylmethylthio)-1,9-dihydro-6H-purin-6-one;
4-[9-ethyl-6-oxo-2-(4-trifluoromethyl-1,3-thiazol-2-ylmethylthio)-6,9-dihydro-1H-purin-1-yl]benzonitrile;
1-(3,4-difluorophenyl)-9-methyl-2-[3,3,3-trifluoropropylthio]-1,9-dihydro-6H-purin-6-one;
1-(3,4-difluorophenyl)-9-methyl-2-[4,4,4-trifluorobutylthio]-1,9-dihydro-6H-purin-6-one;
1-(4-chlorophenyl)-9-methyl-2-(2-trifluoromethyl-1,3-thiazol-4-ylmethylthio)-1,9-dihydro-6H-purin-6-one;
1-(3-fluoro-4-methylphenyl)-9-methyl-2-[3,3,3-trifluoropropylthio]-1,9-dihydro-6H-purin-6-one;
1-(4-chlorophenyl)-9-methyl-2-(4-methylpiperazin-1-yl)-1,9-dihydro-6H-purin-6-one;
1-(4-chloro-2-fluorophenyl)-9-methyl-2-[3,3,3-trifluoropropylthio]-1,9-dihydro-6H-purin-6-one;
1-(4-fluorophenyl)-9-methyl-2-[3,3,3-trifluoropropylthio]-1,9-dihydro-6H-purin-6-one;
1-(4-chloro-3-fluorophenyl)-9-methyl-2-[3,3,3-trifluoropropylthio]-1,9-dihydro-6H-purin-6-one;
1-(4-chloro-3-methoxyphenyl)-9-methyl-2-[3,3,3-trifluoropropylthiol]-1,9-dihydro-6H-puirin-6-one;
2-{1-(4-chlorophenyl)-9-ethyl-6-oxo-6,9-dihydro-1H-purin-2-ylthio}-N-methylacetamide;
2-{1-(4-chlorophenyl)-9-ethyl-6-oxo-6,9-dihydro-1H-purin-2-ylthio}-N,N-diethylacetamide;
1-(4-chlorophenyl)-9-ethyl-2-{5-methylisoxazol-3-ylmethylthio}-1,9-dihydro-6H-purin-6-one;
1-(4-chlorophenyl)-9-methyl-2-methylthio-1,9-dihydro-6H-purin-6-one;
1-(4-fluorophenyl)-9-methyl-2-(4-trifluoromethyl-1,3-thiazol-2-yl]methylthio)-1,9-dihydro-6H-purin-6-one;
9-ethyl-1-(4-fluorophenyl)-2-(4-trifluoromethyl-1,3-thiazol-2-ylmethylthio)-1,9-dihydro-6H-purin-6-one;
1-(3-bromo-4-chlorophenyl)-9-methyl-2-[3,3,3-trifluoropropylthio]-1,9-dihydro-6H-purin-6-one;
1-(4-chlorophenyl)-2-cyclohexylamino-9-ethyl-1,9-dihydro-6H-purin-6-one;
1-(4-chlorophenyl)-2-[3,3,3-trifluoropropylthio]-1,9-dihydro-6H-purin-6-one;
1-(4-chlorophenyl)-9-ethyl-2-{2-methyl-1,3-thiazol-4-ylmethylamino}-1,9-dihydro-6H-purin-6-one;
3-(4-chlorophenyl)-2-(2-trifluoromethyl-1,3-thiazol-4-ylmethylthio)pyrido[3,2-d]pyrimidin-4(3H)-one;
3-(4-chlorophenyl)-2-(2-trifluoromethyl-1,3-thiazol-4-ylmethylamino)pyrido[3,2-d]pyrimidin-4(3H)-one;
1-(4-chloro-3-ethoxyphenyl)-9-ethyl-2-[3,3,3-trifluoropropylthio]-1,9-dihydro-6H-purin-6-one;
1-(4-chloro-3-isopropoxyphenyl)-9-ethyl-2-[3,3,3-trifluoropropylthio]-1,9-dihydro-6H-purin-6-one;
1-(4-chlorophenyl)-9-ethyl-2-{4-[3-trifluoromethylpyridin-2-yl]piperazin-1-yl}-1,9-dihydro-6H-purin-6-one;
3-(4-fluorophenyl)-2-(2-trifluoromethyl-1,3-thiazol-4-ylmethyl}thio)pyrido[3,2-d]pyrimidin-4(3H)-one;
6-(4-chlorophenyl)-5-(2-trifluoromethyl-1,3-thiazol-4-ylmethylthio) [1,3]thiazol[5,4-d]pyrimidin-7(6H)-one;
3-(4-chlorophenyl)-2-cyclohexylaminopyrido[3,2-d]pyrimidin-4(3H)-one;
3-(4-chlorophenyl)-2-[3,3,3-trifluoropropylthio]thieno[3,2-d]pyrimidin-4(3H)-one;
1-(4-chlorophenyl)-9-ethyl-2-(2-trifluoromethyl-1,3-thiazol-4-ylmethylamino)-1,9-dihydro-6H-purin-6-one;
9-ethyl-1-(4-fluorophenyl)-2-(2-trifluoromethyl-1,3-thiazol-4-ylmethylthio)-1,9-dihydro-6H-purin-6-one;
3-(4-chlorophenyl)-7-methyl-2-[3,3,3-trifluoropropylthio]thieno[3,2-d]pyrimidin-4(3H)-one;
3-(4-chlorophenyl)-7-methyl-2-(2-[4-trifluoromethylphenyl]ethylamino)thieno[3,2-d]pyrimidin-4(3H)-one;
3-(4-chlorophenyl)-7-methyl-2-[3,3,3-trifluoropropylamino]thieno[3,2-d]pyrimidin-4(3H)-one;
1-(4-chlorophenyl)-2-{2-(2,4-dichlorophenyl)ethylthio}-9-[2-dimethylaminoethyl]-1,9-dihydro-6H-purin-6-one;
1-(4-chlorophenyl)-9-cyclopropyl-2-(2-[2-methoxy-4-trifluoromethylphenyl]ethylthio)-1,9-dihydro-6H-purin-6-one;
1-(4-chlorophenyl)-9-ethyl-2-[methyl(3,3,3-trifluoropropyl)amino]-1,9-dihydro-6H-purin-6-one;
2-[[5-chloro-1-benzothien-3-ylmethyl](methyl)amino]-1-(4-chlorophenyl)-9-ethyl-1,9-dihydro-6 H-purin-6-one; and
2-chloro-5-{9-methyl-6-oxo-2-[3,3,3-trifluoropropylthio]-6,9-dihydro-1H-purin-1-yl}benzonitrile;
or a pharmaceutically acceptable salt or tautomer thereof.

5. A pharmaceutical composition comprising one or more compounds of any one of claims 1-4, or a pharmaceutically acceptable salt or tautomer thereof in association with a pharmaceutically acceptable carrier or excipient.

6. A compound of any one of claims 1-4, or a pharmaceutically acceptable salt or tautomer thereof, for use in treatment of the human or animal body.

7. The use of a compound of any one of claims 1-4, or a pharmaceutically acceptable salt or tautomer thereof for use in the manufacture of a medicament for the treatment or prevention of physiological disorders that may be ameliorated by modulating VR1 activity.

8. The use of a compound of any one of claims 1-4, or a pharmaceutically acceptable salt or tautomer thereof for use in the manufacture of a medicament for the treatment or prevention of a disease or condition in which pain and/or inflammation predominates.

9. A process for the preparation of a compound of claim 1, which comprises:
(A) for compounds of formula I wherein X is S reacting a compound of formula II with a compound of formula III: 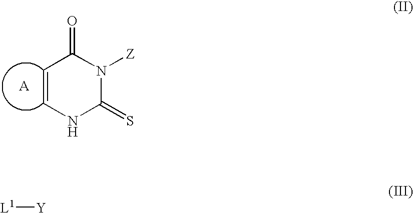
wherein A, Y and Z are as defined in claim 1 and L¹ is a leaving group;
(B) for compounds of formula I wherein X is NR¹ reacting a compound of formula XI with a compound of formula XII: 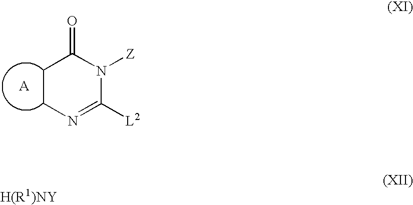
wherein A, R¹, Y and Z are as defined in claim 1 and L² is a leaving group; or
(C) for compounds of formula I wherein X is O reacting a compound of formula XI with a compound of formula XVI:
HOY (XVI)
wherein Y is as defined in claim 1.

10. A method for the treatment or prevention of physiological disorders that may be ameliorated by modulating VR1 activity, which method comprises administration to a patient in need thereof of an effective amount of a compound of claim 1 or a composition comprising a compound of claim 1.

11. A method for the treatment or prevention of a disease or condition in which pain and/or inflammation predominates, which method comprises administration to a patient in need thereof of an effective amount of a compound of claim 1 or a composition comprising a compound of claim 1.
